(19) United States
(12) Patent Application Publication
(10) Pub. No.: US 2021/0059718 A1
(43) Pub. Date: Mar. 4, 2021

(54) INSTRUMENTS, SYSTEMS, AND METHODS FOR MEDICAL PROCEDURES

(71) Applicant: Joseph R. Karam, San Antonio, TX (US)

(72) Inventor: Joseph R. Karam, San Antonio, TX (US)

(21) Appl. No.: 16/934,290

(22) Filed: Jul. 21, 2020

Related U.S. Application Data
(60) Provisional application No. 62/895,480, filed on Sep. 3, 2019.

(51) Int. Cl. A61B 17/34; A61B 17/00; A61M 5/158; A61M 5/142

(52) U.S. Cl. CPC A61B 17/3478; A61B 17/00234; A61M 5/158; A61M 2210/1021; A61B 17/3423; A61M 2202/048; A61M 5/142

(57) ABSTRACT

Systems and instruments for medical procedures and methods relating thereto, such as, for example, those that can be used in minimally invasive medical procedures, among other procedures.

### CROSS-REFERENCE TO RELATED APPLICATIONS

This application claims the benefit of and priority to U.S. Provisional Patent Application Ser. No. 62/895,480, filed Sep. 3, 2019, the entirety of which is incorporated by reference.

### BACKGROUND

### 1. Field of the Invention

The present invention relates generally to instruments, systems, and methods for medical procedures and, more specifically, but not by way of limitation, to systems, instruments, and methods for minimally invasive medical procedures.

### 2. Description of Related Art

Examples of instruments related to medical procedures are disclosed, for example, in U.S. Pat. No. 5,456,684.

### SUMMARY

This disclosure includes embodiments of systems, instruments, and methods for medical procedures, including minimally invasive medical procedures. The systems, instruments, and methods of this disclosure provide various advantages in medical procedures, including, but not limited to, increasing accuracy and/or success rate of medical procedures or portions of medical procedures and increasing visibility of and access to one or more target areas of a medical procedure, which can, among other things, reduce pain during recovery and, therefore, reduce recovery time, reduce cost of the medical procedure, and reduce waste of medical equipment and medication.

Some embodiments include an instrument comprising a head; a shaft comprising a lumen, the shaft in fluid communication with the head, where the shaft is configured to be coupled to and in fluid communication with a fluid reservoir; where at least a portion of the instrument is configured to be disposed through a trocar into a body of a subject; and where, if the instrument is coupled to a fluid reservoir and at least a portion of the instrument is disposed through a trocar into a body of a subject, the instrument is configured to permit delivery of fluid through tissue of a subject.

Some embodiments include a system comprising an instrument, comprising a head; a shaft coupled to the head, the shaft comprising a lumen and tubing extending through the lumen of the shaft and into the head such that the shaft and the head are in fluid communication; a housing coupled to the shaft; where the instrument is configured to enable a fluid reservoir and the head to be in fluid communication; where at least a portion of the instrument is configured to be disposed through a trocar into a body of a subject; and where, if the head is in fluid communication with a fluid reservoir and at least a portion of the instrument is disposed through a trocar into a body of a subject, the instrument is configured to permit delivery of fluid through tissue of a subject.

Some embodiments include an instrument comprising a head configured to be coupled in fluid communication with a needle; a shaft coupled in fluid communication with the head; and a housing coupled in fluid communication with the shaft such that the housing and the head are in fluid communication, where the housing is configured to enable a fluid reservoir to be in fluid communication with the shaft; where the instrument is configured to be coupled to a platform for minimally invasive procedures and controllable by an operator of the platform for minimally invasive procedures; where at least a portion of the instrument is configured to be disposed through a trocar into a body of a subject; and where, if at least a portion of the instrument is disposed through a trocar into a body of a subject, if a fluid reservoir is in fluid communication with the shaft, and if the head is coupled to a needle, the instrument is configured to permit delivery of fluid from the fluid reservoir, through the shaft, the head, and the needle, and through tissue of a subject.

Some embodiments include an instrument comprising a needle; a head configured to be in fluid communication with the needle; a shaft coupled in fluid communication with the head such that, if the head is in fluid communication with the needle, the needle and the shaft are in fluid communication; and a housing coupled in fluid communication with the shaft such that, if the head is in fluid communication with the needle, the housing and the needle are in fluid communication, where the instrument is configured to enable a fluid reservoir to be in fluid communication with the shaft; where at least a portion of the instrument is configured to be disposed through a trocar into a body of a subject; configured to be coupled to a platform for minimally invasive procedures and controllable by an operator of the platform for minimally invasive procedures; and at least one of configured to enable fluid and material to be removed from the body of a subject through the head by a suction device; and configured to enable fluid to move into the body of a subject through the head by an irrigation device; where, if at least a portion of the instrument is disposed through a trocar into a body of a subject, if a fluid reservoir is in fluid communication with the shaft, and if the head is in fluid communication with the needle, the instrument is configured to permit delivery of fluid from the fluid reservoir, through the shaft, the head, and the needle, and through tissue of a subject.

Some embodiments include an instrument comprising a needle; a head comprising a tissue grasper having a first jaw; and a second jaw; where at least one of the first jaw and the second jaw are movable to enable grasping and releasing of tissue of a subject; a shaft coupled the head and configured to be in fluid communication with the needle; and a housing coupled to and in fluid communication with the shaft, where, if the shaft is in fluid communication with the needle, the housing is in fluid communication with the needle; where the instrument is configured to enable a fluid reservoir to be in fluid communication with the shaft; where at least a portion of the instrument is configured to be disposed through a trocar into a body of a subject; and configured to be coupled to a platform for minimally invasive procedures and controllable by an operator of the platform for minimally invasive procedures; and where, if at least a portion of the instrument is disposed through a trocar into a body of a subject, if a fluid reservoir is in fluid communication with the shaft, and if the shaft is in fluid communication with the needle, the instrument is configured to permit delivery of fluid from the fluid reservoir, through the shaft, through the needle, and through tissue of a subject.

Some embodiments include an instrument comprising a needle; a head comprising a suture grasper having a first jaw; and a second jaw; where at least one of the first jaw and the second jaw are movable to enable grasping and releasing of a device for suturing tissue of a subject; a shaft coupled the head and configured to be in fluid communication with the needle; and a housing coupled to and in fluid communication with the shaft, where, if the shaft is in fluid communication with the needle, the housing is in fluid communication with the needle; where the instrument is configured to enable a fluid reservoir to be in fluid communication with the shaft; where at least a portion of the instrument is configured to be disposed through a trocar into a body of a subject; and configured to be coupled to a platform for minimally invasive procedures and controllable by an operator of the platform for minimally invasive procedures; and where, if at least a portion of the instrument is disposed through a trocar into a body of a subject, if a fluid reservoir is in fluid communication with the shaft, and if the shaft is in fluid communication with the needle, the instrument is configured to permit delivery of fluid from the fluid reservoir, through the shaft, through the needle, and through tissue of a subject.

Some embodiments include an instrument comprising a needle; a head comprising a tissue cutting device having a first blade; and a second blade; where at least one of the first blade and the second blade are movable to enable cutting of the tissue of a subject; a shaft coupled the head and configured to be in fluid communication with the needle; and a housing coupled to and in fluid communication with the shaft, where, if the shaft is in fluid communication with the needle, the housing is in fluid communication with the needle; where the instrument is configured to enable a fluid reservoir to be in fluid communication with the shaft; where at least a portion of the instrument is configured to be disposed through a trocar into a body of a subject; and configured to be coupled to a platform for minimally invasive procedures and controllable by an operator of the platform for minimally invasive procedures; and where, if at least a portion of the instrument is disposed through a trocar into a body of a subject, if a fluid reservoir is in fluid communication with the shaft, and if the shaft is in fluid communication with the needle, the instrument is configured to permit delivery of fluid from the fluid reservoir, through the shaft, through the needle, and through tissue of a subject.

Some embodiments include a method for minimally invasive procedures comprising creating at least one incision in a body of a subject; disposing at least a portion of a trocar within the at least one incision to provide access to a target area within the body of the subject; disposing at least a portion of an instrument within the trocar such that the instrument can access the target area within the body of the subject, where the instrument comprises a head; and a shaft in fluid communication with a fluid reservoir, where the fluid reservoir comprises fluid, and at least a portion of the fluid in the fluid reservoir is medication; where the instrument is configured to permit delivery of fluid from the fluid reservoir, through the shaft, and through tissue of the subject; delivering fluid from the fluid reservoir, through the shaft, and through tissue of the target area with the instrument.

Some embodiments include a method for minimally invasive procedures comprising creating at least one incision in a body of a subject; disposing at least a portion of a trocar within the at least one incision to provide access to a target area within the body of the subject; disposing at least a portion of an instrument within the trocar such that the instrument can access the target area within the body of the subject, where the instrument comprises a head; a shaft in fluid communication with a fluid reservoir, where the fluid reservoir comprises fluid; and where the instrument is configured to permit delivery of fluid from the fluid reservoir, through the shaft, and through tissue of the subject; where the instrument is configured to permit removal of fluid from the target area of a subject through tissue of the subject; and where the instrument is configured to be coupled to a platform for minimally invasive procedures; removing fluid from the target area of a subject through tissue of the subject; and delivering fluid from the fluid reservoir, through the shaft, and through tissue of the target area with the instrument.

Some embodiments include a method for minimally invasive procedures comprising creating at least one incision in a body of a subject; disposing at least a portion of a trocar within the at least one incision to provide access to a target area within the body of the subject; coupling an instrument to a platform for minimally invasive procedures to enable an operator to control the instrument; disposing at least a portion of the instrument within the trocar such that the instrument can access the target area within the body of the subject, where the instrument comprises a head in fluid communication with a needle; and a shaft in fluid communication with the head and further in fluid communication with a fluid reservoir, where the fluid reservoir comprises fluid, and at least a portion of the fluid in the fluid reservoir is medication; where the instrument is configured to permit delivery of fluid from the fluid reservoir, through the shaft, through the needle, and into the target area of the subject; delivering fluid from the fluid reservoir, through the shaft, through the needle, and into the target area of the subject with the instrument.

Any embodiment of any of the present instruments, systems, and methods can consist of or consist essentially of—rather than comprise/include/contain/have—any of the described elements and/or features. Thus, in any of the claims, the term “consisting of” or “consisting essentially of” can be substituted for any of the open-ended linking verbs recited above in order to change the scope of a given claim from what it would otherwise be using the open-ended linking verb.

The feature or features of one embodiment may be applied to any other embodiment, even though not described or illustrated, unless expressly prohibited by this disclosure or the nature of the embodiments.

Details associated with the embodiments described above and others are presented below.

### DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. At least some figures illustrate described elements using graphical symbols that will be understood by those of ordinary skill in the art. Components and features of the figures are not drawn to scale. For example, at least some figures depict two waving solid lines representing a break in the depiction of a feature to indicate that such feature can extend in the same manner indefinitely between such waving solid lines and/or can extend at least as long as necessary between such waving solid lines in order to effect the described embodiment(s).

FIG. 1 depicts a perspective view of one embodiment of the present systems and instruments.

FIG. 2 depicts a side view of a portion of the system and instrument of FIG. 1.

FIG. 3 depicts a side view of the portion of the system and instrument of FIG. 2.

FIG. 4 depicts a side view of another embodiment of a portion of the present systems and instruments.

FIG. 5 depicts a side view of the portion of the system and instrument of FIG. 3.

FIG. 6 depicts an example of the present systems and instruments coupled to a portion of a platform for minimally invasive procedures.

FIGS. 7-8 depict a perspective view of another embodiment of the present systems and instruments.

FIGS. 9-13 depict perspective views of portions of various embodiments of the present systems and instruments.

FIG. 14 depicts a perspective view of another embodiment of the present systems and instruments.

FIG. 15-18 depict perspective views of portions of various embodiments of the present systems and instruments.

FIGS. 19-24 depict side views of portions of various embodiments of the present systems and instruments.

FIGS. 25-27 depict cross-sectional views of portions of various embodiments of the present systems and instruments.

FIGS. 28-32 depict perspective views of portions of various embodiments of the present systems and instruments.

### DESCRIPTION OF THE PREFERRED EMBODIMENT

Illustrative embodiments of the tools and methods are described below. In the interest of clarity, all features of an actual implementation may not be described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The terms “a” and “an” are defined as one or more unless this disclosure explicitly requires otherwise.

The term “substantially” is defined as largely, but not necessarily wholly, what is specified (and includes what is specified; e.g., substantially 90 degrees includes 90 degrees), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Terms such as “first” and “second” are used only to differentiate features and not to limit the different features to a particular order or to a particular quantity.

Where numerical ranges or limitations are expressly stated, such express ranges or limitations should be understood to include iterative ranges or limitations of like magnitude falling within the expressly stated ranges or limitations (e.g., from about 1 to about 10 includes 2, 3, 4, etc.; greater than 0.10 includes 0.11, 0.12, 0.13, etc.). For example, whenever a numerical range with a lower limit, Rₗ, and an upper, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Any numerical range defined by two R numbers as defined in the above is also specifically disclosed and includes the two R numbers.

Use of the term “optionally” with respect to any element of a claim means that the element is required, or alternatively, the element is not required, both alternatives being within the scope of the claim.

The terms “comprise” (and any form of comprise, including derivatives, such as “comprises” and “comprising”), “have” (and any form of have, including derivatives, such as “has” and “having”), “include” (and any form of include, including derivatives, such as “includes” and “including”) and “contain” (and any form of contain, including derivatives, such as “contains” and “containing”) are open-ended linking verbs. As a result, an instrument or system of this disclosure that “comprises,” “has,” “includes,” or “contains” one or more elements or features possesses those one or more elements or features, but is not limited to possessing only those elements or features. Likewise, a method that “comprises,” “has,” “includes,” or “contains” one or more steps possesses those one or more steps, but is not limited to possessing only those one or more steps. Use of broader terms such as comprises, includes, and has (and any derivatives of such terms, such as comprising, including, and having) should be understood to provide support for narrower terms, such as consisting of, consisting essentially of, and comprised substantially of. Thus, in any of the claims, the term “consisting of” “consisting essentially of,” or “comprised substantially of” can be substituted for any of the open-ended linking verbs recited above in order to change the scope of a given claim from what it would otherwise be using the open-ended linking verb.

Further, instruments, systems, and components thereof that are “configured” or “configurable” in a certain way are configured or configurable in at least that way, but can also be configured or configurable in other ways than those specifically described.

The term “coupled” is defined as connected, although not necessarily directly, and not necessarily mechanically. Two items are “couplable” if they can be coupled to each other. Unless the context explicitly requires otherwise, items that are couplable are also decouplable, and vice-versa. One non-limiting way in which a first structure is couplable to a second structure is for the first structure to be configured to be coupled to the second structure.

The term “target area” should be understood to include any area that is being targeted, selected, and/or chosen, whether directly or indirectly, to be influenced and/or affected by the systems, instruments, and methods of this disclosure. The term “target area” can include, but is not limited to, areas of a human body, areas of an animal body, and/or areas of any other subject upon which the systems, instruments, and methods of this disclosure are being used. In some instances, though not required, the term “target area” can be defined by a given procedure, by the actions of an operator of the systems, instruments, and methods of this disclosure, and/or by any other factor that encourages use of or results in use of the systems, instruments, and methods of this disclosure in a particular way, in a particular area, or for a particular reason. In some instances, a given procedure can include multiple target areas, some of which are not related to the primary objective of the procedure. For example, if a primary objective of a procedure is to repair an area interior to a body of a subject, and the procedure requires an incision to permit access to the area interior to the body of the subject, the target area can include both the area interior to the body of the subject and the incision site.

The same or similar features of one or more embodiments are sometimes referred to with the same reference numerals within a figure or among figures. However, one or more features having the same reference numeral should not be construed to indicate that any feature is limited to the characteristics of another feature having the same reference numeral, or that any feature cannot already have, or cannot be modified to have, features that are different from another feature having the same reference numeral.

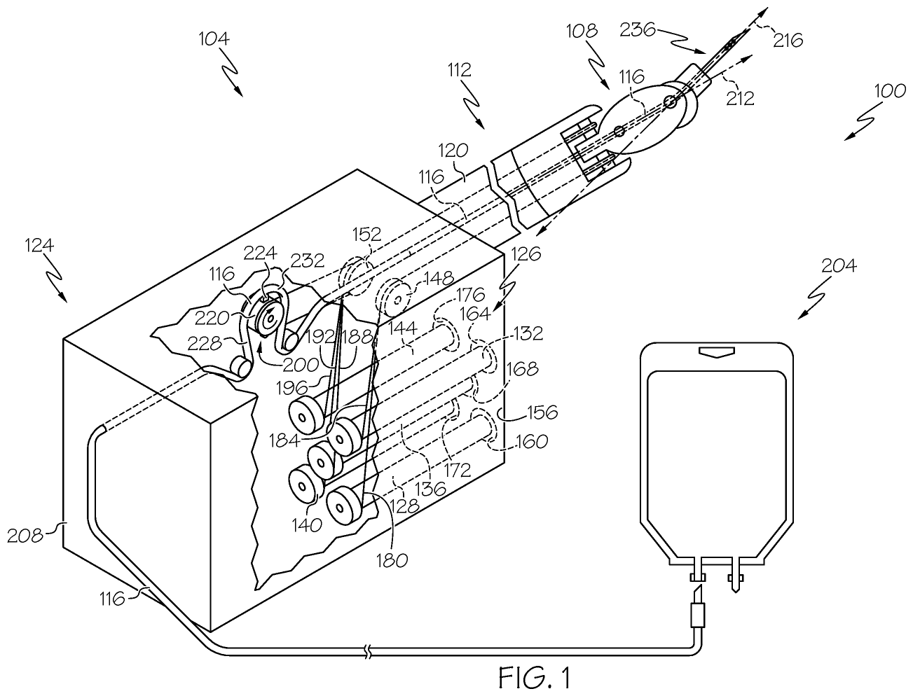

FIGS. 1-32 depict various examples of the instruments, systems, methods, and components thereof that form part of this disclosure. For example, FIG. 1 depicts system 100 comprising instrument 104. In the embodiment shown in FIG. 1, system 100 comprises head 108, which is configured to be coupled to shaft 112 and is depicted coupled to shaft 112 in the embodiment shown. In FIG. 1, shaft 112 is depicted with two wavy solid lines representing a break in the depiction of shaft 112 to indicate that shaft 112 can extend in the same manner indefinitely between such wavy solid lines and/or can extend at least as long as necessary between such wavy solid lines in order to effect the described embodiments. Therefore, at least some of shaft 112 is not drawn to scale. Head 108 can be coupled to shaft 112 in any suitable way, including in any suitable way to enable movement of head 108 with respect to shaft 112, such as, for example, by one or more pins, screws, hinges, adhesive, bolts, and the like. In some embodiments, head 108 is decouplable from shaft 112 to enable replacement of head 108 with a head having similar functionality and/or replacement of head 108 with a head having different functionality. In other embodiments, head 108 is unitary with shaft 112 (e.g., at least partially formed of a single piece of material and, therefore, not couplable to or decouplable from shaft 112).

In the embodiment shown in FIG. 1, head 108 is further coupled to shaft 112 by tubing 116, which extends through lumen 120 of shaft 112 and through head 108 such that shaft 112 and head 108 are in fluid communication. Tubing 116 includes sets of two wavy solid lines depicting breaks in the depiction of tubing 116 to indicate that tubing 116 can extend in the same manner indefinitely between each set of such wavy solid lines and/or can extend at least as long as necessary between each set of such wavy solid lines in order to effect the described embodiments. Therefore, at least some of tubing 116 is not drawn to scale. Portions of tubing of the present disclosure, including tubing 116, can be plastic tubing, metal tubing, rubber tubing, disposable tubing, reusable tubing, combinations thereof, and/or any other tubing that meets medical industry requirements and/or standards for medical or pharmaceutical related applications. Portions of tubing of the present disclosure, including tubing 116, can be flexible tubing in order to, for example, enable bending within instrument 104 or around components of system 100. Portions of tubing of the present disclosure, including tubing 116, can further be rigid in order to, for example, enable movement of one or more components that are coupled to and/or in fluid communication with the tubing by movement of the tubing (e.g., because such rigid tubing may assist in maintaining one or more components that are coupled to and/or in fluid communication with the tubing in substantially fixed relation to the tubing). Portions of tubing of the present disclosure, including tubing 116, can have a diameter of one or more sizes to enable movement of fluid through the tubing, such as, for example, less than 1 millimeter, 1 millimeter, 2 millimeters, 3 millimeters, 4 millimeters, 5 millimeters, 6 millimeters, 7 millimeters, 8 millimeters, 9 millimeters, 10 millimeters, or greater than 10 millimeters.

In the embodiment shown in FIG. 1, shaft 112 is configured to be coupled to housing 124 and is depicted extending from and coupled to housing 124 in the embodiment shown. In the embodiment shown in FIG. 1, head 108 is further coupled to shaft 112 by instrument control system 126, at least a portion of which is disposed within housing 124. In the embodiment shown, instrument control system 126 comprises control shaft 128, control shaft 132, control shaft 136, control shaft 140, control shaft 144, pulley 148, and pulley 152. In some embodiments, instrument control system 126 can comprise less than five control shafts (e.g., four control shafts, three control shafts, two control shafts, or one control shaft); and, in other embodiments, instrument control system 126 can comprise greater than five control shafts (e.g., six control shafts, seven control shafts, eight control shafts, or more). Similarly, in some embodiments, instrument control system 126 can comprise less than two pulleys (e.g., one pulley); and, in other embodiments, instrument control system 126 can comprise greater than two pulleys (e.g., three pulleys, four pulleys, five pulleys, six pulleys, seven pulleys, eight pulleys, or more). In some embodiments, such as in the embodiment shown, instrument control system 126 comprises less pulleys than control shafts; however, in other embodiments, instrument control system 126 can comprise an equal number of pulleys and control shafts or more pulleys than control shafts.

Instrument control systems, such as instrument control system 126, are configured to control various aspects of the present instruments, such as instrument 104. For example, in the embodiment shown in FIG. 1, each of control shafts 128, 132, 136, 140, and 144 extend from and are rotatably coupled to wall 156 of housing 124. Each of control shafts 128, 132, 136, 140, and 144 further comprises control shaft engagement surface 160, 164, 168, 172, and 176, respectively, to enable a device, such as a platform for minimally invasive procedures, to be coupled to and/or engaged with each of control shafts 128, 132, 136, 140, and 144, respectively. In the embodiment shown in FIG. 1, if a device, such as a platform for minimally invasive procedures, is coupled to and/or engaged with at least one of control shaft engagement surfaces 160, 164, 168, 172, and 176 (and, therefore, coupled to and/or engaged with at least one of control shafts 128, 132, 136, 140, and 144, respectively), the at least one of control shaft engagement surfaces 160, 164, 168, 172, and 176 can be rotated by the device (clockwise and/or counterclockwise) in order to rotate a corresponding at least one of control shafts 128, 132, 136, 140, and 144, respectively. For example, if a device, such as a platform for minimally invasive procedures, is coupled to and/or engaged with control shaft engagement surface 160 (and, therefore, coupled to and/or engaged with control shaft 128), control shaft engagement surface 160 can be rotated by the device clockwise (e.g., by an operator) in order to rotate control shaft 128 clockwise and can be rotated by the device counterclockwise (e.g., by an operator) in order to rotate control shaft 128 counterclockwise. Each of control shafts 128, 132, 136, 140, and 144 can be rotated independently via control shaft engagement surfaces 160, 164, 168, 172, and 176, respectively, or simultaneously with one or more of control shafts 128, 132, 136, 140, and 144. The device, such as a platform for minimally invasive procedures, can be controllable by an operator of the device to enable the operator to control instrument 104 via instrument control system 126.

Instrument control systems, such as instrument control system 126 and, more specifically, control shafts, such as control shafts 128, 132, 136, 140, and 144, can be configured to control various aspects of the present instruments, such as instrument 104. For example, in the embodiment shown in FIG. 1, wire 180 (which can represent one wire or a system of wires) is coupled to control shaft 128, engages pulley 148, extends through lumen 120 of shaft 112, and is coupled to head 108. In the embodiment shown in FIG. 1, if a device, such as a platform for minimally invasive procedures, is coupled to and/or engaged with control shaft engagement surface 160 (and, therefore, coupled to and/or engaged with control shaft 128), control shaft engagement surface 160 can be rotated by the device (e.g., by an operator) in order to rotate control shaft 128 such that wire 180 winds about control shaft 128 to tighten the portion of wire 180 that engages pulley 148, extends through lumen 120 of shaft 112, and is coupled to head 108 to enable at least a portion of head 108 to be moved (or, alternatively, such that wire 180 unwinds about control shaft 128 to loosen the portion of wire 180 that engages pulley 148, extends through lumen 120 of shaft 112, and is coupled to head 108 to enable at least a portion of head 108 to be moved).

In the embodiment shown in FIG. 1, wire 184 (which can represent one wire or a system of wires) is coupled to control shaft 132, engages pulley 148, extends through lumen 120 of shaft 112, and is coupled to head 108. In the embodiment shown in FIG. 1, if a device, such as a platform for minimally invasive procedures, is coupled to and/or engaged with control shaft engagement surface 164 (and, therefore, coupled to and/or engaged with control shaft 132), control shaft engagement surface 164 can be rotated by the device in order to rotate control shaft 132 such that wire 184 winds about control shaft 132 to tighten the portion of wire 184 that engages pulley 148, extends through lumen 120 of shaft 112, and is coupled to head 108 to enable at least a portion of head 108 to be moved (or, alternatively, such that wire 184 unwinds about control shaft 132 to loosen the portion of wire 184 that engages pulley 148, extends through lumen 120 of shaft 112, and is coupled to head 108 to enable at least a portion of head 108 to be moved).

In the embodiment shown in FIG. 1, wire 188 (which can represent one wire or a system of wires) is coupled to control shaft 136, engages pulley 152, extends through lumen 120 of shaft 112, and is coupled to head 108. In the embodiment shown in FIG. 1, if a device, such as a platform for minimally invasive procedures, is coupled to and/or engaged with control shaft engagement surface 168 (and, therefore, coupled to and/or engaged with control shaft 136), control shaft engagement surface 168 can be rotated by the device (e.g., by an operator) in order to rotate control shaft 136 such that wire 188 winds about control shaft 136 to tighten the portion of wire 188 that engages pulley 152, extends through lumen 120 of shaft 112, and is coupled to head 108 to enable at least a portion of head 108 to be moved (or, alternatively, such that wire 188 unwinds about control shaft 136 to loosen the portion of wire 188 that engages pulley 152, extends through lumen 120 of shaft 112, and is coupled to head 108 to enable at least a portion of head 108 to be moved).

In the embodiment shown in FIG. 1, wire 192 (which can represent one wire or a system of wires) is coupled to control shaft 140, engages pulley 152, extends through lumen 120 of shaft 112, and is coupled to head 108. In the embodiment shown in FIG. 1, if a device, such as a platform for minimally invasive procedures, is coupled to and/or engaged with control shaft engagement surface 172 (and, therefore, coupled to and/or engaged with control shaft 140), control shaft engagement surface 172 can be rotated by the device (e.g., by an operator) in order to rotate control shaft 140 such that wire 192 winds about control shaft 140 to tighten the portion of wire 192 that engages pulley 152, extends through lumen 120 of shaft 112, and is coupled to head 108 to enable at least a portion of head 108 to be moved (or, alternatively, such that wire 192 unwinds about control shaft 140 to loosen the portion of wire 192 that engages pulley 152, extends through lumen 120 of shaft 112, and is coupled to head 108 to enable at least a portion of head 108 to be moved).

In the embodiment shown in FIG. 1, wire 196 (which can represent one wire or a system of wires) is coupled to control shaft 144, engages pulley 152, and is coupled to pump 200. In the embodiment shown in FIG. 1, if a device, such as a platform for minimally invasive procedures, is coupled to and/or engaged with control shaft engagement surface 176 (and, therefore, coupled to and/or engaged with control shaft 144), control shaft engagement surface 176 can be rotated by the device (e.g., by an operator) in order to rotate control shaft 144 such that wire 196 winds about control shaft 144 to tighten the portion of wire 196 that engages pulley 152 (or, alternatively, unwinds about control shaft 144 to loosen the portion of wire 196 that engages pulley 152) to enable pump 200 to be activated.

In other embodiments, any of control shaft engagement surfaces 160, 164, 168, 172, and 176 may be coupled to a corresponding control shaft (such as control shafts 128, 132, 136, 140, and 144, respectively, as depicted in the embodiment shown) or, alternatively, may not be coupled to a corresponding control shaft, and may be rotated (e.g., by a device, such as a platform for minimally invasive procedures) to control various other aspects of the present instruments, such as instrument 104, including to count a number of times that an instrument has been used (e.g., to predict and/or recommend an expiration for the instrument).

Instrument control system 126 and the various instrument control systems described and depicted in this disclosure are examples of ways in which the present systems can be configured to enable control of the present instruments by an operator. However, it should be understood that a variety of other electrical and/or mechanical systems and configurations can be implemented to achieve the same or similar functionalities described and depicted in this disclosure. Therefore, the components and/or functionality of the present instrument control systems should not be understood to limit the ways in which the present systems and instruments can be configured to be controllable by an operator.

In some embodiments, head 108 is fixed, non-movable, and non-rotatable with respect to shaft 112. In the embodiment shown in FIG. 1, head 108 is configured to be movable with respect to shaft 112. For example, head 108 and shaft 112 may be substantially colinear (e.g., a substantial number of points of each of head 108 and shaft 112 lie in the same line or linear sequence) and head 108 is configured to be movable with respect to shaft 112 such that head 108 (or at least a portion of head 108) is non-colinear with respect to shaft 112 (e.g., at least some points of head 108 do not lie in the same line or linear sequence as at least some points of shaft 112). For example, as depicted in FIG. 1, line 212 is substantially coaxial with shaft 112, and line 216 is substantially coaxial with at least a portion of head 108. In the embodiment shown, lines 212 and 216 are substantially non-colinear and represent an embodiment in which head 108 and shaft 112 are substantially non-colinear. In the embodiment shown, head 108 is configured to be movable such that lines 212 and 216 are substantially colinear (and, therefore, represents an embodiment in which head 108 and shaft 112 are substantially colinear). In some embodiments, head 108 is configured to be rotatable with respect to shaft 112 while head 108 and shaft 112 remain substantially colinear (e.g., head 108 may be rotatable 360, 720, 1080 degrees or more while shaft 112 remains stationary). In such embodiments, head 108 can comprise at least one degree of freedom with respect to shaft 112 (e.g., one degree of freedom, two degrees of freedom, three degrees of freedom, four degrees of freedom, five degrees of freedom, and/or six degrees of freedom).

In the embodiment shown in FIG. 1, instrument 104 is configured to enable fluid reservoir 204 and head 108 to be in fluid communication (e.g., via housing 124 and, more specifically, via tubing 116 within housing 124). For example, instrument 104 is configured such that head 108 is coupled to and in fluid communication with fluid reservoir 204 via housing 124 (and, more specifically, tubing 116 extending through housing 124), and shaft 112 is coupled to and in fluid communication with fluid reservoir 204 via housing 124 (and, more specifically, tubing 116 extending through housing 124). In the embodiment shown, fluid reservoir 204 is a collapsible bag (e.g., an intravenous fluid bag). In other embodiments, fluid reservoir 204 can be a syringe, a vial, and/or any other container configured to accommodate fluid. For example, tubing 116 extends from head 108, through lumen 120 of shaft 112, through housing 124, and exits wall 208 of housing 124 to enable fluid reservoir 204 and head 108 to be in fluid communication via housing 124. In some embodiments, tubing 116 comprises a fixed connection with housing 124 and/or fluid reservoir 204; and in other embodiments, tubing 116 is couplable to and/or decouplable from housing 124. Tubing 116 can be couplable to and/or decouplable from housing 124 and fluid reservoir 204 by any suitable connection, such as, for example, a screw connection, a snap connection, a Luer taper connection, a spike connection in which a piercing device is coupled to a portion of tubing 116 and can be pierced through a portion of fluid reservoir 204 or housing 124, and similar connections. Tubing 116 is configured to be coupled to fluid reservoir 204 to enable fluid from fluid reservoir 204 to exit fluid reservoir 204 and move from fluid reservoir 204, into and through housing 124, through lumen 120 of shaft 112, and into head 108. Tubing 116 can extend from fluid reservoir 204 to head 108 in one or more pieces of tubing.

Fluid reservoir 204 can include fluid for delivery into a body of a subject, such as, for example, water, saline, medication (e.g., anesthetics, vasoconstrictors, and/or reducing agents, such as, lidocaine, prilocaine, tetracaine, bupivacaine, lontocaine, septocaine, cocaine, benzocaine, chloroprocaine, cyclomethycaine, dimethocaine, piperocaine, propoxycaine, procaine, proparacaine, articaine, cinchocaine, etidocaine, levobupivacaine, mepivacaine, ropivacaine, trimecaine, saxitoxin, neosaxitoxin, tetrodotoxin, menthol, eugenol, spilanthol, adrenaline, epinephrine, and combinations thereof), and/or fluid that is detectable within a body of a subject with a viewing device (e.g., a medical imaging device, such as a camera or endoscope, an ultrasound, and the like). Fluid that is detectable within a body of a subject with a viewing device may be detectable, for example, by containing an ingredient that responds to the electromagnetic spectrum differently from surrounding tissues, such as an ingredient that responds to (e.g., reflects) far infrared, mid infrared, near infrared, near ultraviolet, extreme ultraviolet, soft x-rays, hard x-rays, gamma rays, combinations thereof, and/or other portions of the electromagnetic spectrum that may respond differently than body tissues.

In some embodiments, if fluid reservoir 204 contains fluid, fluid exits fluid reservoir 204 and can move into housing 124, through tubing 116 extending through lumen 120 of shaft 112, and into head 108 due at least in part to force of gravity. In some embodiments, if fluid reservoir 204 contains fluid, fluid exits fluid reservoir 204 and moves into housing 124, through tubing 116 extending through lumen 120 of shaft 112, and into head 108 due at least in part to force applied to fluid reservoir 204 by an operator (e.g., by applying manual pressure to fluid reservoir 204, such as by squeezing fluid reservoir 204, to assist fluid in exiting fluid reservoir 204). In the embodiment shown, if fluid reservoir 204 contains fluid, instrument 104 (and, more specifically, pump 200) is further configured to pump and/or encourage fluid from fluid reservoir 204 into housing 124, through tubing 116 extending through lumen 120 of shaft 112, and into head 108. In the embodiment shown, pump 200 is disposed within housing 124; however, in other embodiments, pump 200 can be disposed exterior to housing 124 while still being configured to pump fluid from fluid reservoir 204 into housing 124, through tubing 116 extending through lumen 120 of shaft 112, and into head 108.

In the embodiment shown, pump 200 is a positive displacement pump. Pump 200 comprises cylinder 220 and knob 224. Cylinder 220 is coupled to wire 196 such that cylinder 220 rotates clockwise if control shaft 144 is rotated such that wire 196 winds about control shaft 144 to tighten the portion of wire 196 that engages pulley 152. In other embodiments, instrument control system 126 can be configured to rotate cylinder 220 clockwise if control shaft 144 is rotated such that wire 196 unwinds about control shaft 144 to loosen the portion of wire 196 that engages pulley 152. Knob 224 contacts tubing 116 and rotates with cylinder 220. As cylinder 220 rotates, knob 224 creates expanding cavity 228 on a portion of tubing 116 that extends from fluid reservoir 204 and contracting cavity 232 on a portion of tubing 116 that extends toward head 108, causing fluid from fluid reservoir 204 to move into expanding cavity 228 of tubing 116 and causing fluid to exit contracting cavity 232 of tubing 116. With each full rotation of cylinder 220, a constant volume of fluid moves through expanding cavity 228 and contracting cavity 232 of tubing 116. Though depicted as one knob 224, instrument 104 can comprise more than one knob 224 (e.g., one knob, two knobs, three knobs, four knobs, or more) in order to encourage fluid to move through tubing 116. Tubing 116 (and, more specifically, expanding cavity 228 and contracting cavity 232) can be adjusted (e.g., by adjusting length and/or diameter of expanding cavity 228 and/or adjusting length and/or diameter of contracting cavity 232) to enable a predetermined amount of fluid (and/or a substantially equal volume of fluid in succession) to move through pump 200 and into tubing 116 extending through lumen 120 of shaft 116 with each rotation of cylinder 220. In other embodiments, pump 200 can be a mechanical pump, an electrical pump, a pneumatic pump, an impulse pump, a gravity pump, and any combination thereof. Pump 200 can further be coupled to a power source (e.g., batteries or an electrical source) that is configured to provide power to pump 200 when activated.

In the embodiment shown in FIG. 1, system 100 further comprises needle 236 extending from head 108. In some embodiments, head 108 can be coupled to (or can be configured to be coupled to) needle 236. Needle 236 is in fluid communication with tubing 116 extending through head 108 and, therefore, needle 236 is in fluid communication with tubing 116 extending through housing 124. If instrument 104 is coupled to fluid reservoir 204, needle 236 is in fluid communication with fluid reservoir 204. In the embodiment shown in FIG. 1, if pump 200 is activated (e.g., by rotating control shaft engagement surface 176) and if fluid reservoir 204 is coupled to instrument 104, fluid moves from fluid reservoir 204, into housing 124, through shaft 112, and into needle 236 of head 108.

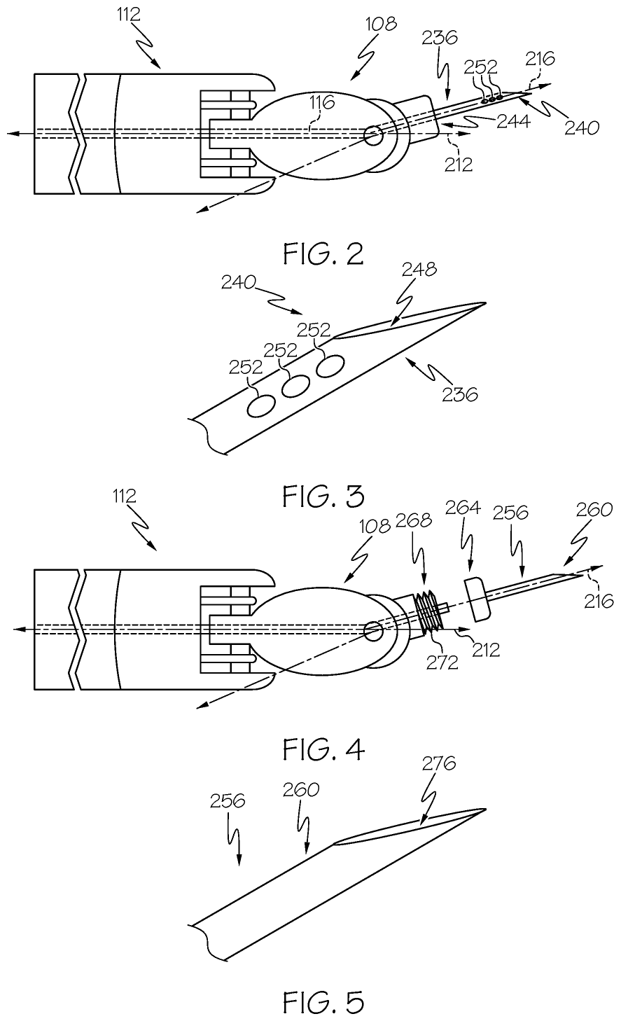

FIGS. 2-3 depict a portion of instrument 104 of FIG. 1. More specifically, FIG. 2 depicts a portion of shaft 112 coupled to head 108, which is unitary with needle 236 (e.g., at least partially formed of a single piece of material such that needle 236 is not decouplable from head 108), and FIG. 3 depicts a portion of needle 236. Needle 236 comprises first end 240 and second end 244. Second end 244 of needle 236 is in fluid communication with tubing 116. First end 240 of needle 236 comprises end opening 248, which is configured to permit fluid (e.g., such as fluid from fluid reservoir 204) to exit needle 236. In some embodiments, first end 240 of needle 236 can be configured to prevent fluid from exiting needle 236 (e.g., such as if first end 240 of needle 236 does not comprise end opening 248 or if end opening 248 is blocked to prevent fluid from exiting needle 236). In the embodiment shown in FIGS. 2-3, needle 236 further comprises at least one opening 252 (and, more specifically, three openings 252) between first end 240 and second end 244 of needle 236, each of which is configured to permit fluid to exit needle 236. In some embodiments, needle 236 can have more than three openings 252 (e.g., four openings, five openings, six openings, seven openings, eight openings, or more); and, in other embodiments, needle 236 can have less than three openings 252 (e.g., two openings or one opening).

FIG. 4 depicts a portion of instrument 104 of FIG. 1 with another example of a needle of the present disclosure. More specifically, FIG. 4 depicts a portion of shaft 112 coupled to head 108, which is configured to be coupled to needle 256, and FIG. 5 depicts a portion of needle 256. Needle 256 comprises first end 260 and second end 264. Second end 264 of needle 256 is configured to be coupled to (and decoupled from) needle attachment end 268 of head 108. In the embodiment shown, needle attachment end 268 comprises threads 272 that are configured to engage with corresponding threads of second end 264 of needle 256 to enable head 108 to be coupled to and decoupled from needle 256. In other embodiments, second end 264 of needle 256 can be coupled to (and, in some embodiments, decoupled from) head 108 by any suitable connection, including a snap connection, an adhesive, a Luer taper, and the like. First end 260 of needle 256 comprises end opening 276, which is configured to permit fluid (e.g., such as fluid from fluid reservoir 204) to exit needle 256.

Aspects of any of the embodiments described above or below may be combined with aspects of any of the other embodiments described in this disclosure to form further embodiments having comparable or different properties and addressing the same or different problems. Similarly, the benefits and advantages described above or below may relate to one embodiment or may relate to several embodiments. For example, the embodiment shown or described with respect to FIGS. 2-3 can be modified, adapted, and/or supplemented to comprise any of the features shown or described with respect to FIGS. 4-5; and, similarly, the embodiment shown or described with respect to FIGS. 4-5 can be modified, adapted, and/or supplemented to comprise any of the features shown or described with respect to FIGS. 2-3. As an example, head 108 and needle 236 of FIG. 2 can be configured to be coupled to each other to enable needle 236 to be coupled to and, in some embodiments, decoupled from head 108. As another example, needle 256 of FIG. 2 can be configured to have at least one opening between first end 260 and second end 264 configured to permit fluid to exit needle 256. It should be understood that any embodiment described or depicted in this disclosure can be modified, adapted, and/or supplemented to include any other feature described or depicted in this disclosure.

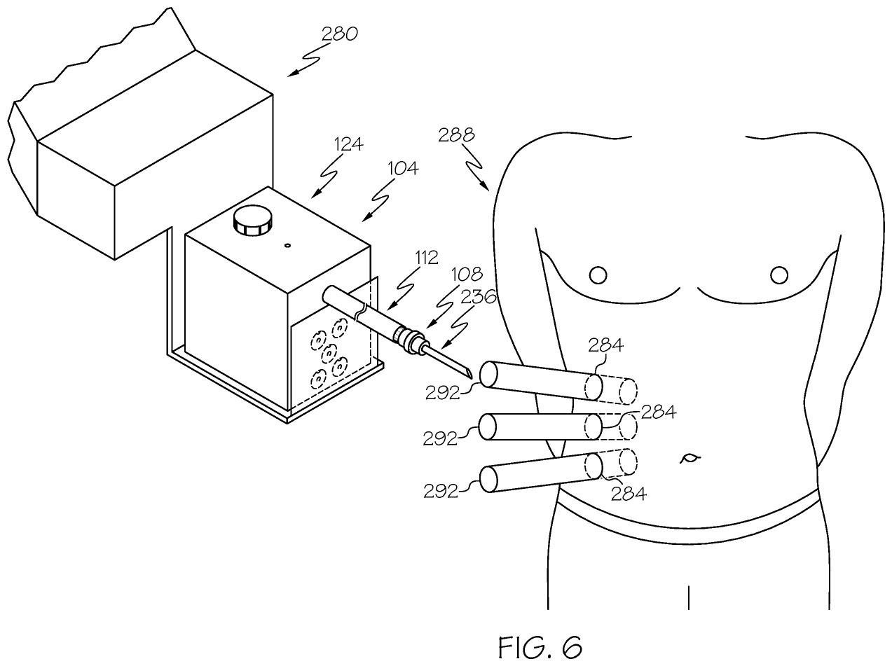

The systems, instruments, and components related thereto depicted and described in this disclosure may be used in minimally invasive procedures. For example, as depicted in FIG. 6, system 100 (and, more specifically, instrument 104) can be coupled to a platform for minimally invasive procedures (a portion of which is depicted in FIG. 6 as arm 280) in order to be controllable by an operator of a device/platform for minimally invasive procedures. The portion of a platform for minimally invasive procedures depicted in FIG. 6 is one example of a portion of such a platform and is not drawn to scale. The devices and platforms for minimally invasive procedures referenced throughout this disclosure can include any electrical and/or mechanical device couplable to a system and/or instrument and controllable by an operator that enables an operator to perform a surgical procedure in a manner that is minimally invasive to a subject. An “operator” can include a physician (e.g., a surgeon), a surgical assistant, staff of an operating room, computer software (e.g., such as artificially intelligent computer software or computer software that is preprogramed to respond to various stimuli or circumstances), combinations thereof, and/or any person or thing providing input into the device, platform, systems, and/or instruments disclosed herein. Any embodiment in this disclosure may include one or more operators to effect the described embodiment. For example, to the extent that the operator is a human operator, the operator can be positioned at a point of control (e.g., or console) for a platform for minimally invasive surgery, and such point of control is configured to translate one or more inputs (e.g., physical inputs, vocal/audio inputs, electrical inputs (e.g., including any mental inputs), and the like) into actions of the platform and, ultimately, into actions of any system or instrument coupled to the device or platform. An operator can provide input to the device or platform in a variety of ways, including through foot switch pedal controls, finger clutch controls, hand controls, vocal/audio controls, mental controls, and/or other modalities for surgical techniques, including minimally invasive surgical techniques. Some input into a device, platform, system, or instrument of this disclosure may be from a surgical assistant or staff in the operating room who may have access to a portion of the device, platform, system, and/or instrument that a primary operator may not otherwise be able to access. As previously discussed, some devices or platforms may include software that is configured to provide input to the device or platform. Some software can be programmed to respond to stimuli or circumstances encountered in a given surgical procedure, or to suggest or encourage action based on such stimuli or circumstances. Other software can be programmed to interpret stimuli or circumstances in an artificially intelligent manner and to respond based on such interpretations. An instrument coupled to the device or platform may offer feedback (e.g., or can create a feedback control loop) to the software to provide more information to the software for future actions. In circumstances involving such examples of computer software, a surgical procedure may not be entirely under the control of a human operator; however, both a human operator and a software operator should be understood to be considered an “operator” in combination.

In the embodiment show, if head 108 is configured to be coupled to a needle, such as with needle 256 as depicted in FIGS. 4-5, a needle can be coupled to head 108 such that the needle is in fluid communication with tubing 116 extending through head 108, lumen 120 of shaft 112, and housing 124. Fluid reservoir 204 can be coupled to tubing 116 of instrument 104 such that fluid reservoir 204 and head 108 (and, more specifically, needle 236 of FIGS. 2-3 or needle 256 of FIGS. 4-5) are in fluid communication.

One or more incision (e.g., three incision 284) can be made in a body of a subject (e.g., body 288), such as in the abdomen, and a trocar (e.g., trocars 292) can be disposed in each incision (e.g., each of incisions 284) to provide access to the interior of the body of the subject (e.g., interior to body 288). At least a portion of instrument 104, such as needle 236 or needle 256, head 108, and a portion of shaft 112 is configured to be disposed through a trocar (e.g., one of trocars 292) into a body of a subject (e.g., body 288), and a platform for minimally invasive procedures (e.g., such as arm 280) can be docked to a trocar (e.g., one or more of trocars 292). If disposed through a trocar (e.g., one of trocars 292) into a body of a subject (e.g., body 288), instrument 104 is configured to permit delivery of fluid through tissue of a subject. For example, in some embodiments, tissue of a subject can be pierced by needle 236 or needle 256 to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292) into a body of a subject (e.g., body 288), instrument 104 is further configured to permit removal of fluid through tissue of a subject. For example, in some embodiments, tissue of a subject can be pierced by needle 236 or needle 256 to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 204 can exit fluid reservoir 204 due at least in part to gravity, force applied to fluid reservoir 204, and/or activation of pump 200 (e.g., via a device, such as a platform for minimally invasive procedures, or via housing 124). Fluid from fluid reservoir 204 exits fluid reservoir 204 through tubing 116, enters housing 124, shaft 112, head 108, and, if an operator of the platform for minimally invasive procedures has directed instrument 104 (and, more specifically, needle 236 or needle 256) to pierce tissue of a subject, fluid exits needle 236 or needle 256 into tissue of a subject or a lumen of an organ of the subject.

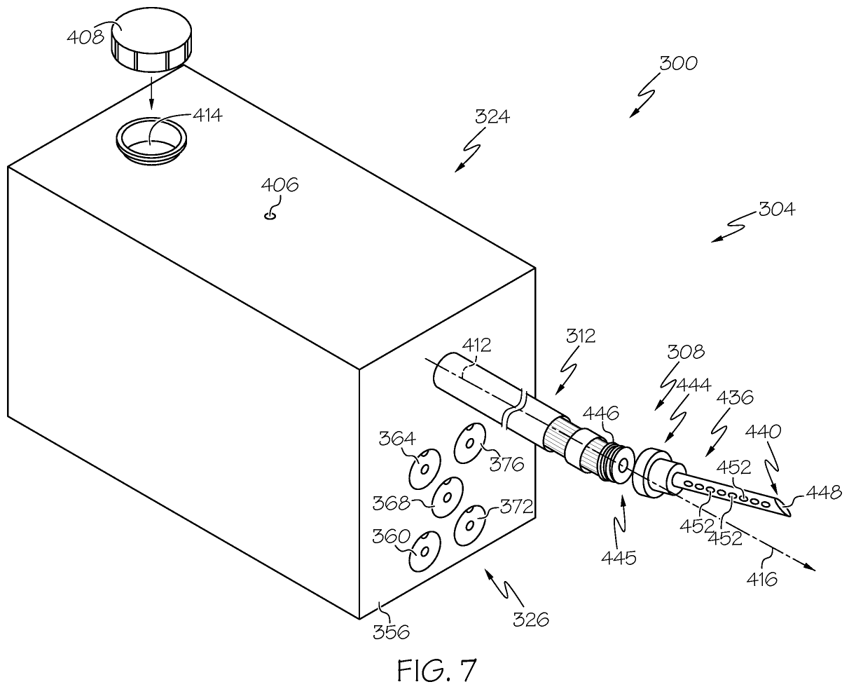

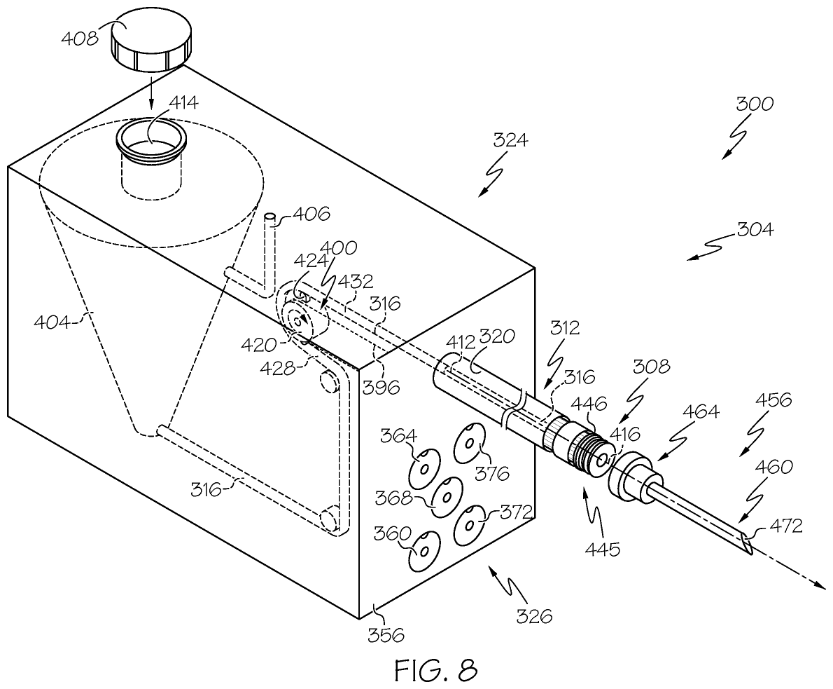

FIGS. 7-8 depict additional examples of the present instruments, systems, methods, and components thereof. As explained above, aspects of any of the embodiments depicted or described above or below may be combined with aspects of the embodiments depicted and described with respect to FIGS. 7-8 to form further embodiments having comparable or different properties and addressing the same or different problems. The embodiments depicted in FIGS. 7-8 and described below can be modified, adapted, and/or supplemented to comprise any of the features shown or described with respect to any of the other systems, instruments, or methods in this disclosure. Similarly, embodiments depicted in other figures or described with respect thereto can be modified, adapted, and/or supplemented to comprise any of the features shown or described with respect to the systems, instruments, and methods of FIGS. 7-8. One or more features of FIGS. 7-8 having the same or similar reference numeral as other features of other embodiments in this disclosure should not be construed to indicate that any feature is limited to the characteristics of another feature having the same or similar reference numeral, or that any feature cannot already have, or cannot be modified to have, features that are different from another feature having the same or similar reference numeral. Though the same or similar feature may have a detailed description with respect to a first embodiment, a second embodiment having the same or similar feature without such a detailed description of such feature (or without any description of such feature) should not be understood to preclude such feature of the second embodiment from comprising the same or similar characteristics as depicted or described with respect to the feature of the first embodiment. For the sake of brevity, all features that an embodiment has or could have may not be described in each separate embodiment.

FIGS. 7-8 depict system 300 comprising instrument 304. In the embodiments shown in FIGS. 7-8, system 300 comprises head 308, which is configured to be coupled to shaft 312 and is depicted coupled to shaft 312 in the embodiments shown. In FIGS. 7-8, shaft 312 is depicted with two wavy solid lines representing a break in the depiction of shaft 312 to indicate that shaft 312 can extend in the same manner indefinitely between such wavy solid lines and/or can extend at least as long as necessary between such wavy solid lines in order to effect the described embodiments. Therefore, at least some of shaft 312 is not drawn to scale. Head 308 can be coupled to shaft 312 in any suitable way, including in any suitable way to enable movement of head 308 with respect to shaft 312, such as, for example, by one or more pins, screws, hinges, adhesive, bolts, and the like. In some embodiments, head 308 is decouplable from shaft 312 to enable replacement of head 308 with a head having similar functionality and/or replacement of head 308 with a head having different functionality. In other embodiments, head 308 is unitary with shaft 312 (e.g., at least partially formed of a single piece of material and, therefore, not couplable to or decouplable from shaft 312).

In the embodiments shown in FIGS. 7-8, head 308 is further coupled to shaft 312 by tubing 316, which extends through lumen 320 of shaft 312 and through head 308 such that shaft 312 and head 308 are in fluid communication. Tubing 316 includes two wavy solid lines depicting breaks in the depiction of tubing 316 to indicate that tubing 316 can extend in the same manner indefinitely between such wavy solid lines and/or can extend at least as long as necessary between such wavy solid lines in order to effect the described embodiments. Therefore, at least some of tubing 316 is not drawn to scale. Tubing 316 can comprise the same or similar features and characteristics as those depicted and described with respect to tubing 116.

In the embodiments shown in FIGS. 7-8, shaft 312 is configured to be coupled to housing 324 and is depicted extending from and coupled to housing 324 in the embodiments shown. In the embodiments shown in FIGS. 7-8, head 308 is further coupled to shaft 312 by instrument control system 326, at least a portion of which is disposed within housing 324 and at least a portion of which is depicted in FIGS. 7-8. Instrument control system 326 can comprise the same or similar features and characteristics that operate in the same or similar way as those depicted and described with respect to instrument control system 126, including instrument control shafts and pulleys, which are not depicted in FIGS. 7-8. For example, housing 324 of instrument 304 comprises wall 356 and control shaft engagement surfaces 360, 364, 368, 372, and 376, which are rotatably coupled to wall 356. Each of control shaft engagement surfaces 360, 364, 368, 372, and 376 can be rotated in order to rotate a corresponding control shaft within housing 324 to control various aspects of instrument 304, as described in detail above, to enable an operator to control instrument 304, including moving head 308 with respect to shaft 312 (e.g., such as an operator of a device, including a platform for minimally invasive procedures).

In the embodiment shown in FIG. 8, wire 396 (which can represent one wire or a system of wires) is coupled to a control shaft and pulley of instrument control system 326 and is further coupled to pump 400. In the embodiment shown in FIG. 8, if a device, such as a platform for minimally invasive procedures, is coupled to and/or engaged with control shaft engagement surface 376 (and, therefore, coupled to and/or engaged with a control shaft), control shaft engagement surface 376 can be rotated by the device (e.g., by an operator) in order to rotate the corresponding control shaft such that wire 396 winds about the control shaft to tighten the portion of wire 396 (e.g., about one or more pulleys) to enable pump 400 to be activated.

In some embodiments, head 308 is fixed, non-movable, and non-rotatable with respect to shaft 312. In the embodiments shown in FIGS. 7-8, head 308 is configured to be movable with respect to shaft 312. For example, head 308 and shaft 312 may be substantially colinear (e.g., a substantial number of points of each of head 308 and shaft 312 lie in the same line or linear sequence) and head 308 is configured to be movable with respect to shaft 312 such that head 308 (or at least a portion of head 308) is non-colinear with respect to shaft 312 (e.g., at least some points of head 308 do not lie in the same line or linear sequence as at least some points of shaft 312). For example, as depicted in FIGS. 7-8, line 412 is substantially coaxial with shaft 312, and line 416 is substantially coaxial with at least a portion of head 308. In the embodiments shown, lines 312 and 316 are substantially colinear and represent an embodiment in which head 308 and shaft 312 are substantially colinear. In the embodiments shown, head 308 is configured to be movable such that lines 312 and 316 are substantially non-colinear (and, therefore, represents an embodiment in which head 308 and shaft 312 are substantially non-colinear). In some embodiments, head 308 is configured to be rotatable with respect to shaft 312 while head 308 and shaft 312 remain substantially colinear (e.g., head 308 may be rotatable 360, 720, 1080 degrees or more while shaft 312 remains stationary). In such embodiments, head 308 can comprise at least one degree of freedom with respect to shaft 312 (e.g., one degree of freedom, two degrees of freedom, three degrees of freedom, four degrees of freedom, five degrees of freedom, and/or six degrees of freedom).

In the embodiments shown in FIGS. 7-8, instrument 304 is configured to enable fluid reservoir 404 and head 308 to be in fluid communication (e.g., via housing 324 and, more specifically, via tubing 316 within housing 324). In the embodiments shown in FIGS. 7-8, housing 324 comprises fluid reservoir 404. In the embodiments shown, fluid reservoir 404 is unitary with housing 324 (e.g., fluid reservoir 404 is substantially rigid and at least partially formed of the same piece of material as housing 324 such that fluid reservoir 404 cannot be removed from housing 324 and/or such that fluid reservoir 404 forms a cavity within housing 324). In some embodiments, housing 324 can be configured such that fluid reservoir 404 can be coupled to and decoupled from housing 304. In some embodiments, the fluid reservoir can be a collapsible bag (e.g., an intravenous fluid bag) disposed within a portion of housing 324 and/or coupled to the cavity in housing 324 that presently represents fluid reservoir 404. Tubing 316 is in fluid communication with fluid reservoir 404 to enable fluid reservoir and head 308 to be in fluid communication. In some embodiments, tubing 316 comprises a fixed connection with fluid reservoir 404; and in other embodiments, tubing 316 is couplable to and/or decouplable from fluid reservoir 404. Tubing 316 can be couplable to and/or decouplable from fluid reservoir 404 by any suitable connection, such as, for example, a screw connection, a snap connection, a Luer taper connection, a spike connection in which a piercing device is coupled to a portion of tubing 316 and can be pierced through a portion of fluid reservoir 324, and similar connections. Tubing 316 can extend from fluid reservoir 404 to head 308 in one or more pieces of tubing. Fluid within fluid reservoir 324 can comprise any fluid described above with respect to fluid reservoir 204. Housing 324 further comprises pressure release tubing 406 and cap 408. Cap 408 is configured to be coupled to housing 324 (e.g., by a screw connection, a snap connection, and the like) to seal opening 414 in order to prevent fluid from exiting fluid reservoir 404 through seal opening 414. Pressure release tubing 406 is in fluid communication with fluid reservoir 404 and is coupled to housing 324 such that fluid reservoir 404 and the atmosphere exterior to housing 324 can be in fluid communication. In some embodiments, fluid reservoir 404 and the atmosphere are in fluid communication via pressure release tubing 406. In other embodiments, at least one of fluid reservoir 404, pressure release tubing 406, and housing 324 comprises a valve (e.g., a one-way valve or a two-way valve). If cap 408 is coupled to housing 324, and if fluid exits fluid reservoir 404 via tubing 316, a vacuum is created in fluid reservoir 404 such that pressure in fluid reservoir 404 is less than atmospheric pressure. A valve disposed in at least one of fluid reservoir 404, pressure release tubing 406, and housing 324 is configured to permit equalization of atmospheric pressure with pressure in fluid reservoir 404 to continue to permit fluid to exit fluid reservoir 404 into tubing 316.

If fluid reservoir 404 contains fluid, instrument 304 (and, more specifically, pump 400) is configured to pump and/or encourage fluid from fluid reservoir 404 into housing 324, through tubing 316 extending through lumen 320 of shaft 312, and into head 308. In the embodiments shown, pump 400 is disposed within housing 324; however, in other embodiments, pump 400 can be disposed exterior to housing 324 while still being configured to pump fluid from fluid reservoir 404 into housing 324, through tubing 316 extending through lumen 320 of shaft 312, and into head 308.

In the embodiments shown, pump 400 is a positive displacement pump. Pump 400 comprises cylinder 420 and knob 424. Cylinder 420 is coupled to wire 396 such that cylinder 420 rotates clockwise if control shaft engagement surface 376 is rotated. Knob 424 contacts tubing 316 and rotates with cylinder 420. As cylinder 420 rotates, knob 424 creates expanding cavity 428 on a portion of tubing 316 that extends from fluid reservoir 404 and contracting cavity 432 on a portion of tubing 316 that extends toward head 308, causing fluid from fluid reservoir 404 to move into expanding cavity 428 of tubing 316 and causing fluid to exit contracting cavity 432 of tubing 316. With each full rotation of cylinder 420, a constant volume of fluid moves through expanding cavity 428 and contracting cavity 432 of tubing 316. Though depicted as one knob 424, instrument 104 can comprise more than one knob 424 (e.g., one knob, two knobs, three knobs, four knobs, or more) in order to encourage fluid to move through tubing 316. Tubing 316 (and, more specifically, expanding cavity 428 and contracting cavity 432) can be adjusted (e.g., by adjusting length and/or diameter of expanding cavity 428 and/or adjusting length and/or diameter of contracting cavity 432) to enable a predetermined amount of fluid (and/or a substantially equal volume of fluid in succession) to move through pump 400 and into tubing 316 extending through lumen 320 of shaft 316 with each rotation of cylinder 420. In other embodiments, pump 400 can be a mechanical pump, an electrical pump, a pneumatic pump, an impulse pump, a gravity pump, and any combination thereof. Pump 400 can further be coupled to a power source (e.g., batteries or an electrical source) that is configured to provide power to pump 400 when activated.

Variations in needle are depicted in FIGS. 7 and 8; however, various other components of system 300 remain the same or similar and, therefore, are referred to using reference numerals previously described, though not always depicted in both of FIG. 7 and FIG. 8. In the embodiment shown in FIG. 7, system 300 comprises needle 436, which is configured to be coupled to (and decoupled from) head 308. In other embodiments, head 308 can be unitary with needle 436 (e.g., at least partially formed of a single piece of material such that needle 436 is not decouplable from head 308). If needle 436 is coupled to head 308, needle 436 is in fluid communication with tubing 316 extending through head 308 and, therefore, needle 436 is in fluid communication with tubing 316 extending through housing 324 and coupled to fluid reservoir 404. If pump 400 is activated (e.g., by rotating control shaft engagement surface 376) and if needle 436 is coupled to head 308, fluid moves from fluid reservoir 404, into housing 324, through shaft 312, through head 308, and into needle 436. In the embodiment shown in FIG. 7, needle 436 comprises first end 440 and second end 444. Second end 444 of needle 436 is configured to be in fluid communication with tubing 316. In the embodiment shown, second end 444 of needle 436 is configured to be coupled to needle attachment end 445 of head 308. Needle attachment end 445 of head 308 comprises threads 446 that are configured to engage with corresponding threads of second end 444 of needle 436 to enable head 308 to be coupled to and decoupled from needle 456. In other embodiments, second end 444 of needle 436 can be coupled to (and, in some embodiments, decoupled from) head 308 by any suitable connection, including a snap connection, an adhesive, a Luer taper, and the like. First end 440 of needle 436 comprises end opening 448, which is configured to permit fluid (e.g., such as fluid from fluid reservoir 404) to exit needle 436. In some embodiments, first end 440 of needle 436 can be configured to prevent fluid from exiting needle 436 (e.g., such as if first end 440 of needle 436 does not comprise end opening 448 or if end opening 448 is blocked to prevent fluid from exiting needle 436). In the embodiment shown in FIG. 7, needle 436 further comprises at least one opening 452 (and, more specifically, eight openings 452) between first end 440 and second end 444 of needle 436, each of which is configured to permit fluid to exit needle 436. In some embodiments, needle 436 can have more than eight openings 452 (e.g., nine openings, ten openings, eleven openings, twelve openings, or more); and, in other embodiments, needle 436 can have less than eight openings 452 (e.g., seven openings, six openings, five openings, four openings, three openings, two openings or one opening).

In the embodiment shown in FIG. 8, system 300 comprises needle 456, which is another example of a needle of the present disclosure, and which is configured to be coupled to (and decoupled from) head 308. In other embodiments, head 308 can be unitary with needle 456 (e.g., at least partially formed of a single piece of material such that needle 456 is not decouplable from head 308). If needle 456 is coupled to head 308, needle 456 is in fluid communication with tubing 316 extending through head 308 and, therefore, needle 456 is in fluid communication with tubing 316 extending through housing 324 and coupled to fluid reservoir 404. If pump 400 is activated (e.g., by rotating control shaft engagement surface 376) and if needle 456 is coupled to head 308, fluid moves from fluid reservoir 404, into housing 324, through shaft 312, through head 308, and into needle 456. In the embodiment shown in FIG. 8, needle 456 comprises first end 460 and second end 464. Second end 464 of needle 456 is configured to be in fluid communication with tubing 316. In the embodiment shown, second end 464 of needle 456 is configured to be coupled to needle attachment end 445 of head 308. Needle attachment end 445 of head 308 comprises threads 446 that are configured to engage with corresponding threads of second end 464 of needle 456 to enable head 308 to be coupled to and decoupled from needle 456. In other embodiments, second end 464 of needle 456 can be coupled to (and, in some embodiments, decoupled from) head 308 by any suitable connection, including a snap connection, an adhesive, a Luer taper, and the like. First end 460 of needle 456 comprises end opening 472, which is configured to permit fluid (e.g., such as fluid from fluid reservoir 404) to exit needle 456.

System 300 may be used in minimally invasive procedures. For example, as with the embodiment depicted in FIG. 6, instrument 304 can be coupled to a platform for minimally invasive procedures (an example of which is depicted in FIG. 6 as arm 280) in order to be controllable by an operator of the platform for minimally invasive procedures. A needle (e.g., needle 436 or needle 456) can be coupled to head 308 such that the needle is in fluid communication with tubing 316 extending through head 308, lumen 320 of shaft 312, and housing 324. If fluid reservoir 404 is not already coupled to tubing 316, fluid reservoir 404 can be coupled to tubing 316 of instrument 304 such that fluid reservoir 404 and head 308 (and, more specifically, needle 436 of FIG. 7 or needle 456 of FIG. 8) are in fluid communication. System 300 can be used in connection with a platform for minimally invasive procedures in the same or similar manner as described above with respect to system 100. For example, one or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 304, such as needle 436 or needle 456, head 308, and a portion of shaft 312 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 304 is configured to permit delivery of fluid through tissue of a subject. For example, in some embodiments, tissue of a subject can be pierced by needle 436 or needle 456 to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 304 is further configured to permit removal of fluid through tissue of a subject. For example, in some embodiments, tissue of a subject can be pierced by needle 436 or needle 456 to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 404 can exit fluid reservoir 404 due at least in part to activation of pump 400 (e.g., via a device, such as a platform for minimally invasive procedures, or via housing 324). Fluid from fluid reservoir 404 exits fluid reservoir 404 through tubing 316, enters housing 324, shaft 312, head 308, and, if an operator of the platform for minimally invasive procedures has directed instrument 304 (and, more specifically, needle 436 or needle 456) to pierce tissue of a subject, fluid exits needle 436 or needle 456 into tissue of a subject or a lumen of an organ of the subject.

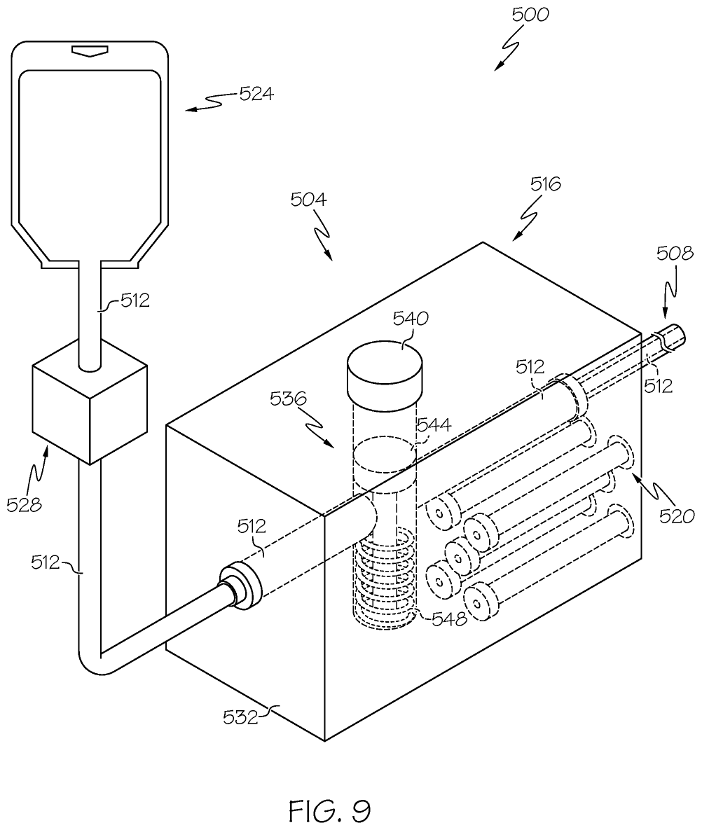

FIGS. 9-13 depict additional examples of the present instruments, systems, methods, and components thereof. As explained above, aspects of any of the embodiments depicted or described above or below may be combined with aspects of the embodiments depicted and described with respect to FIGS. 9-13 to form further embodiments having comparable or different properties and addressing the same or different problems. The embodiments depicted in FIGS. 9-13 and described below can be modified, adapted, and/or supplemented to comprise any of the features shown or described with respect to any of the other systems, instruments, or methods in this disclosure. Similarly, embodiments depicted in other figures or described with respect thereto can be modified, adapted, and/or supplemented to comprise any of the features shown or described with respect to the systems, instruments, and methods of FIGS. 9-13. One or more features of FIGS. 9-13 having the same or similar reference numeral as other features of other embodiments in this disclosure should not be construed to indicate that any feature is limited to the characteristics of another feature having the same or similar reference numeral, or that any feature cannot already have, or cannot be modified to have, features that are different from another feature having the same or similar reference numeral. Though the same or similar feature may have a detailed description with respect to a first embodiment, a second embodiment having the same or similar feature without such a detailed description of such feature (or without any description of such feature) should not be understood to preclude such feature of the second embodiment from comprising the same or similar characteristics as depicted or described with respect to the feature of the first embodiment. For the sake of brevity, all features that an embodiment has or could have may not be described in each separate embodiment.

FIGS. 9-13 depict various examples of housings, fluid reservoirs, pumps, configurations thereof, and mechanisms to permit and/or encourage fluid to move through the depicted systems. Though the systems of FIGS. 9-13 may not depict each feature or component depicted or described with respect to other embodiments, such as heads of instruments, needles, instrument control systems or components thereof, and the like, it should be understood that FIGS. 9-13 can comprise any such components that operate in the same or similar manner as described or depicted in this disclosure.

FIG. 9 depicts system 500 comprising instrument 504. In the embodiment shown in FIG. 9, system 500 comprises shaft 508, a portion of which is depicted in FIG. 9, which is configured to be coupled to a head of the present disclosure. Shaft 508 is depicted with two wavy solid lines representing a break in the depiction of shaft 508 to indicate that shaft 508 can extend in the same manner indefinitely between such wavy solid lines and/or can extend at least as long as necessary between such wavy solid lines in order to effect the described embodiments. Therefore, at least some of shaft 508 is not drawn to scale.

The embodiment shown in FIG. 9 further comprises tubing 512, which extends through shaft 508 and toward a head to which instrument 504 can be coupled such that shaft 508 and a head can be in fluid communication. Tubing 512 includes two wavy solid lines depicting breaks in the depiction of tubing 512 to indicate that tubing 512 can extend in the same manner indefinitely between such wavy solid lines and/or can extend at least as long as necessary between such wavy solid lines in order to effect the described embodiments. Therefore, at least some of tubing 512 is not drawn to scale. Tubing 512 can comprise the same or similar features and characteristics as those depicted and described with respect to tubing 116.

In the embodiment shown in FIG. 9, shaft 508 is configured to be coupled to housing 516 and is depicted extending from and coupled to housing 516 in the embodiments shown. In the embodiment shown in FIG. 9, a head can further be coupled to shaft 508 by instrument control system 520 (or can be indirectly coupled to instrument control system 520), at least a portion of which is disposed within housing 516 and at least a portion of which is depicted in FIG. 9. Instrument control system 520 can comprise the same or similar features and characteristics that operate in the same or similar way as those depicted and described with respect to instrument control system 126, including instrument control shafts, control shaft engagement surfaces, pulleys, and wires, some of which are not depicted in FIG. 9. Instrument control system 520 can be configured to enable an operator to control instrument 504, including moving a head with respect to shaft 508 (e.g., such as an operator of a device, including a platform for minimally invasive procedures).

In the embodiment shown in FIG. 9, instrument 504 is configured to enable fluid reservoir 524 and a head of the present disclosure to be in fluid communication, such as, for example, via tubing 512, which extends from fluid reservoir 524, through pump 528, through housing 516, through shaft 508, and into a head to which instrument 504 can be coupled. In the embodiment shown, fluid reservoir 524 is a collapsible bag (e.g., an intravenous fluid bag). In other embodiments, fluid reservoir 524 can be a syringe, a vial, and/or any other container configured to accommodate fluid. Tubing 512 is configured to be coupled to (and decoupled from) fluid reservoir 524 (and is depicted coupled to fluid reservoir 524, in the embodiment shown). Tubing 512 is further configured to be coupled to (and decoupled from) pump 528 (and is depicted coupled to pump 528, in the embodiment shown). In some embodiments, tubing 512 comprises a fixed connection with pump 528 such that tubing 512 can be coupled to and decoupled from at least one of fluid reservoir 524 and housing 516 but cannot be decoupled from pump 528. Tubing 512 exits pump 528 and enters housing 516 through wall 532 of housing 516. In some embodiments, tubing 512 comprises a fixed connection with wall 532 of housing 516; and in other embodiments, tubing 512 is couplable to and/or decouplable from wall 532 of housing 516. Tubing 512 can be couplable to and/or decouplable from fluid reservoir 524, pump 528, and wall 532 of housing 516 by any suitable connection, such as, for example, a screw connection, a snap connection, a Luer taper connection, a spike connection in which a piercing device is coupled to a portion of tubing 512 and can be pierced through a portion of fluid reservoir 524, pump 528, or wall 532 of housing 516, and similar connections. Tubing 512 can extend from fluid reservoir 524 to a head of instrument 504 in one or more pieces of tubing. Fluid within fluid reservoir 524 can comprise any fluid described above with respect to fluid reservoir 204.

In some embodiments, if fluid reservoir 524 contains fluid, fluid exits fluid reservoir 524 and can move through pump 528 and into housing 516, through tubing 512 extending through shaft 508, and into a head to which instrument 504 can be coupled due at least in part to force of gravity. In some embodiments, if fluid reservoir 524 contains fluid, fluid exits fluid reservoir 524 and can move through pump 528 and into housing 516, through tubing 512 extending through shaft 508, and into a head to which instrument 504 can be coupled due at least in part to force applied to fluid reservoir 524 by an operator (e.g., by applying manual pressure to fluid reservoir 524, such as by squeezing fluid reservoir 524, to assist fluid in exiting fluid reservoir 524). In the embodiment shown, if fluid reservoir 524 contains fluid, instrument 504 (and, more specifically, pump 528) is further configured to pump and/or encourage fluid from fluid reservoir 524 into housing 516, through tubing 512 extending through shaft 508, and into a head to which instrument 504 can be coupled. In the embodiment shown, pump 528 is disposed exterior to housing 516; however, in other embodiments, pump 528 can be disposed within housing 516 while still being configured to pump fluid from fluid reservoir 524 into housing 516, through tubing 512 extending through shaft 508, and into head to which instrument 504 can be coupled. In the embodiment shown, pump 528 can be a positive displacement pump, a mechanical pump, an electrical pump, a pneumatic pump, an impulse pump, a gravity pump, and any combination thereof. Pump 528 can further be coupled to a power source (e.g., batteries or an electrical source) that is configured to provide power to pump 528 when activated.

In the embodiment shown in FIG. 9, instrument 504 and, more specifically, housing 516, further comprises fluid control system 536 that is configured to enable housing 516 to be controllable by an operator to enable fluid to enter shaft 508 and/or to be controllable by an operator to prevent fluid from entering shaft 508. For example, in the embodiment shown, fluid control system 536 comprises button 540, stop 544, and spring 548 coupled to stop 544. If button 540 is engaged by an operator, spring 548 is depressed such that stop 544 moves. In some embodiments, stop 544 can be positioned such that, if button 540 is unengaged, stop 544 does not interfere with and/or block tubing 512 such that fluid is not prevented by stop 544 from entering shaft 508. In such an embodiment, if an operator engages button 540 to move stop 544 to interfere with and/or block tubing 512, fluid is prevented from entering shaft 508. In other embodiments, stop 544 can be positioned such that, if button 540 is unengaged, stop 544 interferes with and/or blocks tubing 512 such that fluid is prevented by stop 544 from entering shaft 508. In such an embodiment, if an operator engages button 540 to move stop 544 so that it does not interfere with and/or block tubing 512, fluid is permitted to move into shaft 508 (e.g., due to a pressure applied on fluid within tubing 512 via gravity, an applied pressure on fluid reservoir 524, and/or pump 528). In some embodiments, button 540 can be controllable by or engageable through a platform for minimally invasive procedures. Fluid control system 536 can be configured in various other ways in order to enable control of fluid within system 500 from housing 516 by an operator. For example, fluid control system 536 may be electrically and/or mechanically coupled to pump 528 such that an operator can engage button 540 and activate pump 528 to pump fluid from fluid reservoir 524 into shaft 508.

System 500 and/or a device to which instrument 504 is coupled, such as a platform for minimally invasive procedures, can be configured to permit delivery of a predetermined amount of fluid (and/or a substantially equal volume of fluid in For example, if an operator of a platform for minimally invasive procedures to which succession). instrument 504 is coupled activates pump 528, pump 528 can pump a predetermined amount of fluid from fluid reservoir 524 into tubing 512 (and, after such amount of fluid has entered tubing 512, pump 528 can deactivate, either by the operator or automatically). As another example, if an operator engages button 540, a predetermined amount of fluid can be permitted to move through tubing 512 past fluid control system 536 and into shaft 508 (and, after such amount of fluid has entered shaft 508, fluid control system 536 can be configured to prevent additional fluid from moving past fluid control system 536).

As discussed above, instrument 504 can comprise any of the head configurations described or depicted in this disclosure that can operate in the same or similar way. In this way, system 500 may be used in minimally invasive procedures. For example, instrument 504 can be coupled to a platform for minimally invasive procedures (similar to that depicted in FIG. 6) in order to be controllable by an operator of the platform for minimally invasive procedures. A head comprising or coupled to a needle can be coupled to shaft 508 such that the head and the needle are in fluid communication with tubing 512 extending through shaft 508 and housing 516 and in fluid communication with fluid reservoir 524.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 504, such as a needle, a head, and a portion of shaft 508 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 504 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 504 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 524 can exit fluid reservoir 524 due at least in part to gravity, force applied to fluid reservoir 524, and/or activation of pump 528 (e.g., via a device, such as a platform for minimally invasive procedures, or via housing 516). Fluid from fluid reservoir 524 exits fluid reservoir 524 through tubing 512, enters housing 516, shaft 508, a head, and a needle, and, if an operator of the platform for minimally invasive procedures has directed instrument 504 (and, more specifically, a needle) to pierce tissue of a subject, fluid exits the needle into tissue of a subject or a lumen of an organ of the subject.

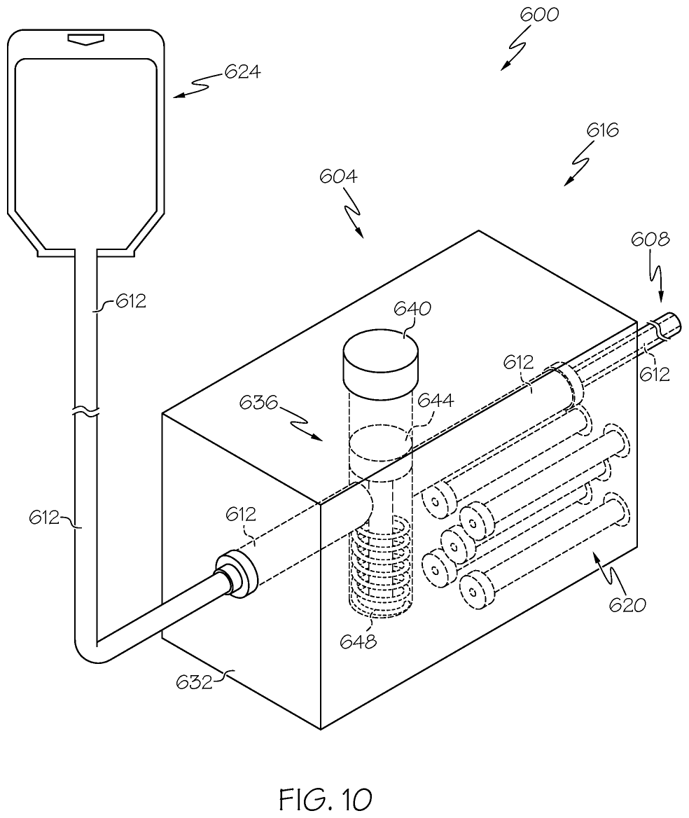

FIG. 10 depicts system 600 comprising instrument 604. In the embodiment shown in FIG. 10, system 600 comprises shaft 608, a portion of which is depicted in FIG. 10, which is configured to be coupled to a head of the present disclosure. Shaft 608 is depicted with two wavy solid lines representing a break in the depiction of shaft 608 to indicate that shaft 608 can extend in the same manner indefinitely between such wavy solid lines and/or can extend at least as long as necessary between such wavy solid lines in order to effect the described embodiments. Therefore, at least some of shaft 608 is not drawn to scale.

The embodiment shown in FIG. 10 further comprises tubing 612, which extends through shaft 608 and toward a head to which instrument 604 can be coupled such that shaft 608 and a head can be in fluid communication. Tubing 612 includes sets of two wavy solid lines depicting breaks in the depiction of tubing 612 to indicate that tubing 612 can extend in the same manner indefinitely between such sets of wavy solid lines and/or can extend at least as long as necessary between such sets of wavy solid lines in order to effect the described embodiments. Therefore, at least some of tubing 612 is not drawn to scale. Tubing 612 can comprise the same or similar features and characteristics as those depicted and described with respect to tubing 116.

In the embodiment shown in FIG. 10, shaft 608 is configured to be coupled to housing 616 and is depicted extending from and coupled to housing 616 in the embodiments shown. In the embodiment shown in FIG. 10, a head can further be coupled to shaft 608 by instrument control system 620 (or can be indirectly coupled to instrument control system 620), at least a portion of which is disposed within housing 616 and at least a portion of which is depicted in FIG. 10. Instrument control system 620 can comprise the same or similar features and characteristics that operate in the same or similar way as those depicted and described with respect to instrument control system 126, including instrument control shafts, control shaft engagement surfaces, pulleys, and wires, some of which are not depicted in FIG. 10. Instrument control system 620 can be configured to enable an operator to control instrument 604, including moving a head with respect to shaft 608 (e.g., such as an operator of a device, including a platform for minimally invasive procedures).

In the embodiment shown in FIG. 10, instrument 604 is configured to enable fluid reservoir 624 and a head of the present disclosure to be in fluid communication, such as, for example, via tubing 612, which extends from fluid reservoir 624, through housing 616, through shaft 608, and into a head to which instrument 604 can be coupled. In the embodiment shown, fluid reservoir 624 is a collapsible bag (e.g., an intravenous fluid bag). In other embodiments, fluid reservoir 624 can be a syringe, a vial, and/or any other container configured to accommodate fluid. Tubing 612 is configured to be coupled to (and decoupled from) fluid reservoir 624 (and is depicted coupled to fluid reservoir 624, in the embodiment shown). Tubing 612 exits fluid reservoir 624 and enters housing 616 through wall 632 of housing 616. In some embodiments, tubing 612 comprises a fixed connection with wall 632 of housing 616; and in other embodiments, tubing 612 is couplable to and/or decouplable from wall 632 of housing 616. Tubing 612 can be couplable to and/or decouplable from fluid reservoir 624 and wall 632 of housing 616 by any suitable connection, such as, for example, a screw connection, a snap connection, a Luer taper connection, a spike connection in which a piercing device is coupled to a portion of tubing 612 and can be pierced through a portion of fluid reservoir 624 or wall 632 of housing 624, and similar connections. Tubing 612 can extend from fluid reservoir 624 to a head of instrument 604 in one or more pieces of tubing. Fluid within fluid reservoir 624 can comprise any fluid described above with respect to fluid reservoir 204.

In the embodiment shown in FIG. 10, if fluid reservoir 624 contains fluid, fluid exits fluid reservoir 624 and can move into housing 616, through tubing 612 extending through shaft 608, and into a head to which instrument 604 can be coupled due at least in part to force of gravity. In the embodiment shown in FIG. 10, if fluid reservoir 624 contains fluid, fluid exits fluid reservoir 624 and can move into housing 616, through tubing 612 extending through shaft 608, and into a head to which instrument 604 can be coupled due at least in part to force applied to fluid reservoir 624 by an operator (e.g., by applying manual pressure to fluid reservoir 624, such as by squeezing fluid reservoir 624, to assist fluid in exiting fluid reservoir 524).

In the embodiment shown in FIG. 10, instrument 604 and, more specifically, housing 616, further comprises fluid control system 636 that is configured to enable housing 616 to be controllable by an operator to enable fluid to enter shaft 608 and/or to be controllable by an operator to prevent fluid from entering shaft 608. For example, in the embodiment shown, fluid control system 636 comprises button 640, stop 644, and spring 648 coupled to stop 644. If button 640 is engaged by an operator, spring 648 is depressed such that stop 644 moves. In some embodiments, stop 644 can be positioned such that, if button 640 is unengaged, stop 644 does not interfere with and/or block tubing 612 such that fluid is not prevented by stop 644 from entering shaft 608. In such an embodiment, if an operator engages button 640 to move stop 644 to interfere with and/or block tubing 612, fluid is prevented from entering shaft 608. In other embodiments, stop 644 can be positioned such that, if button 640 is unengaged, stop 644 interferes with and/or blocks tubing 612 such that fluid is prevented by stop 644 from entering shaft 608. In such an embodiment, if an operator engages button 640 to move stop 644 so that it does not interfere with and/or block tubing 612, fluid is permitted to move into shaft 608 (e.g., due to a pressure applied on fluid within tubing 612 via gravity and/or an applied pressure on fluid reservoir 624). In some embodiments, button 640 can be controllable by or engageable through a platform for minimally invasive procedures.

System 600 and/or a device to which instrument 604 is coupled, such as a platform for minimally invasive procedures, can be configured to permit delivery of a predetermined amount of fluid (and/or a substantially equal volume of fluid in succession). For example, if an operator engages button 640, a predetermined amount of fluid can be permitted to move through tubing 612 past fluid control system 636 and into shaft 608 (and, after such amount of fluid has entered shaft 608, fluid control system 636 can be configured to prevent additional fluid from moving past fluid control system 636).

As discussed above, instrument 604 can comprise any of the head configurations described or depicted in this disclosure that can operate in the same or similar way. In this way, system 600 may be used in minimally invasive procedures. For example, instrument 604 can be coupled to a platform for minimally invasive procedures (similar to that depicted in FIG. 6) in order to be controllable by an operator of the platform for minimally invasive procedures. A head comprising or coupled to a needle can be coupled to shaft 608 such that the head and the needle are in fluid communication with tubing 612 extending through shaft 608 and housing 616 and in fluid communication with fluid reservoir 624.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 604, such as a needle, a head, and a portion of shaft 608 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 604 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 604 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 624 can exit fluid reservoir 624 due at least in part to gravity and/or force applied to fluid reservoir 624, and/or fluid control system 636). Fluid from fluid reservoir 624 exits fluid reservoir 624 through tubing 612, enters housing 616, shaft 608, a head, and a needle, and, if an operator of the platform for minimally invasive procedures has directed instrument 604 (and, more specifically, a needle) to pierce tissue of a subject, fluid exits the needle into tissue of a subject or a lumen of an organ of the subject.

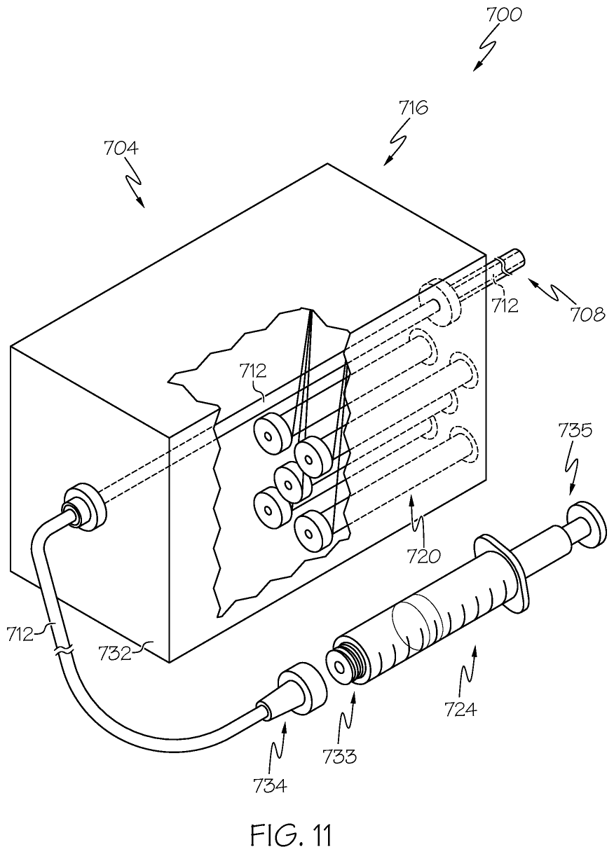

FIG. 11 depicts system 700 comprising instrument 704. In the embodiment shown in FIG. 11, system 700 comprises shaft 708, a portion of which is depicted in FIG. 11, which is configured to be coupled to a head of the present disclosure. Shaft 708 is depicted with two wavy solid lines representing a break in the depiction of shaft 708 to indicate that shaft 708 can extend in the same manner indefinitely between such wavy solid lines and/or can extend at least as long as necessary between such wavy solid lines in order to effect the described embodiments. Therefore, at least some of shaft 708 is not drawn to scale.

The embodiment shown in FIG. 11 further comprises tubing 712, which extends through shaft 708 and toward a head to which instrument 704 can be coupled such that shaft 708 and a head can be in fluid communication. Tubing 712 includes sets of two wavy solid lines depicting breaks in the depiction of tubing 712 to indicate that tubing 712 can extend in the same manner indefinitely between such sets of wavy solid lines and/or can extend at least as long as necessary between such sets of wavy solid lines in order to effect the described embodiments. Therefore, at least some of tubing 712 is not drawn to scale. Tubing 712 can comprise the same or similar features and characteristics as those depicted and described with respect to tubing 116.

In the embodiment shown in FIG. 11, shaft 708 is configured to be coupled to housing 716 and is depicted extending from and coupled to housing 716 in the embodiments shown. In the embodiment shown in FIG. 11, a head can further be coupled to shaft 708 by instrument control system 720 (or can be indirectly coupled to instrument control system 720), at least a portion of which is disposed within housing 716 and at least a portion of which is depicted in FIG. 11. Instrument control system 720 can comprise the same or similar features and characteristics that operate in the same or similar way as those depicted and described with respect to instrument control system 126, including instrument control shafts, control shaft engagement surfaces, pulleys, and wires, some of which are not depicted in FIG. 11. Instrument control system 720 can be configured to enable an operator to control instrument 704, including moving a head with respect to shaft 708 (e.g., such as an operator of a device, including a platform for minimally invasive procedures).

In the embodiment shown in FIG. 11, instrument 704 is configured to enable fluid reservoir 724 and a head of the present disclosure to be in fluid communication. In the embodiment shown, fluid reservoir 724 is configured to be coupled to tubing 712. If fluid reservoir 724 is coupled to tubing 712, fluid reservoir 724 can be in fluid communication with a head to which instrument 704 can be coupled via tubing 712, which extends from fluid reservoir 724, through wall 732 of housing 716, through shaft 708, and into a head to which instrument 704 can be coupled. In the embodiment shown, fluid reservoir 724 is a syringe; however, in other embodiments, fluid reservoir 724 can be a collapsible bag, a vial, and/or any other container configured to accommodate fluid. In some embodiments, tubing 712 comprises a fixed connection with wall 732 of housing 716; and in other embodiments, tubing 712 is couplable to and/or decouplable from wall 732 of housing 716. Tubing 712 can be couplable to and/or decouplable from fluid reservoir 724 and wall 732 of housing 716 by any suitable connection, such as, for example, a screw connection, a snap connection, a Luer taper connection, a spike connection in which a piercing device is coupled to a portion of tubing 712 and can be pierced through a portion of fluid reservoir 724 or wall 732 of housing 724, and similar connections. For example, in the embodiment shown, fluid reservoir 724 comprises threaded end 733, which is configured to engage with corresponding threads of fluid reservoir connector 734 to enable fluid reservoir 724 to be coupled to and in fluid communication with tubing 712. Tubing 712 can extend from fluid reservoir 724 to a head of instrument 704 in one or more pieces of tubing. Fluid within fluid reservoir 724 can comprise any fluid described above with respect to fluid reservoir 204. In the embodiment shown in FIG. 11, if fluid reservoir 724 contains fluid, fluid exits fluid reservoir 724 and can move into housing 716, through tubing 712 extending through shaft 708, and into a head to which instrument 704 can be coupled due at least in part to force applied to fluid reservoir 724 by an operator. For example, an operator can apply manual pressure to fluid reservoir 724 by engaging plunger 735 to assist fluid in exiting fluid reservoir 724 into tubing 712.

System 700 and/or a device to which instrument 704 is coupled, such as a platform for minimally invasive procedures, can be configured to permit delivery of a predetermined amount of fluid (and/or a substantially equal volume of fluid in succession). For example, a predetermined amount of fluid can be drawn into fluid reservoir 724 such that, if an operator engages plunger 735 of fluid reservoir 724, a predetermined amount of fluid is permitted to move through tubing 712 into housing 716 and into shaft 708.

As discussed above, instrument 704 can comprise any of the head configurations described or depicted in this disclosure that can operate in the same or similar way. In this way, system 700 may be used in minimally invasive procedures. For example, instrument 704 can be coupled to a platform for minimally invasive procedures (similar to that depicted in FIG. 6) in order to be controllable by an operator of the platform for minimally invasive procedures. A head comprising or coupled to a needle can be coupled to shaft 708 such that the head and the needle are in fluid communication with tubing 712 extending through shaft 708 and housing 716 and in fluid communication with fluid reservoir 724.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 704, such as a needle, a head, and a portion of shaft 708 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 704 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 704 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 724 can exit fluid reservoir 724 due at least in part to force applied to fluid reservoir 724 (e.g., such as force applied to plunger 735). Fluid from fluid reservoir 724 exits fluid reservoir 724 through tubing 712, enters housing 716, shaft 708, a head, and a needle, and, if an operator of the platform for minimally invasive procedures has directed instrument 704 (and, more specifically, a needle) to pierce tissue of a subject, fluid exits the needle into tissue of a subject or a lumen of an organ of the subject.

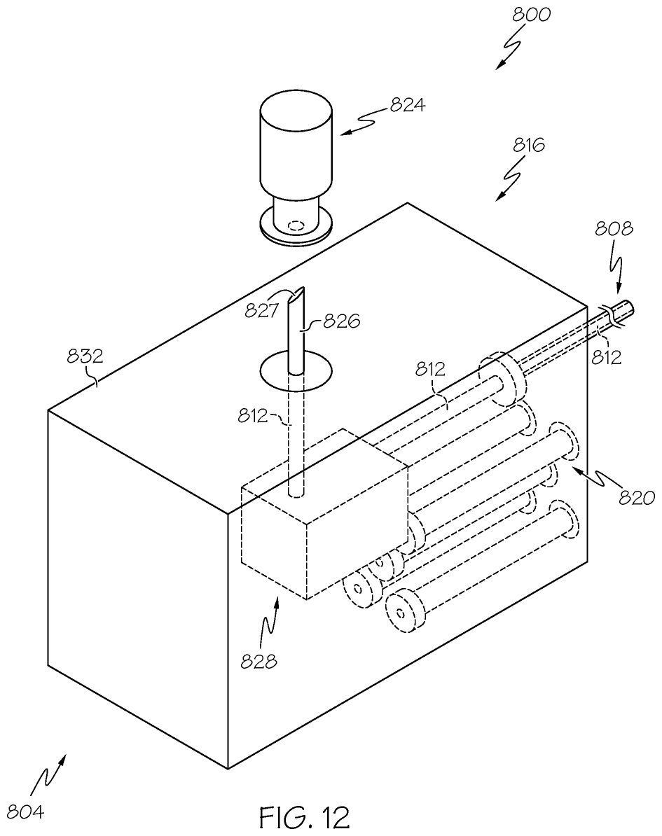

FIG. 12 depicts system 800 comprising instrument 804. In the embodiment shown in FIG. 12, system 800 comprises shaft 808, a portion of which is depicted in FIG. 12, which is configured to be coupled to a head of the present disclosure. Shaft 808 is depicted with two wavy solid lines representing a break in the depiction of shaft 808 to indicate that shaft 808 can extend in the same manner indefinitely between such wavy solid lines and/or can extend at least as long as necessary between such wavy solid lines in order to effect the described embodiments. Therefore, at least some of shaft 808 is not drawn to scale.

The embodiment shown in FIG. 12 further comprises tubing 812, which extends through shaft 808 and toward a head to which instrument 804 can be coupled such that shaft 808 and a head can be in fluid communication. Tubing 812 includes two wavy solid lines depicting breaks in the depiction of tubing 812 to indicate that tubing 812 can extend in the same manner indefinitely between such wavy solid lines and/or can extend at least as long as necessary between such wavy solid lines in order to effect the described embodiments. Therefore, at least some of tubing 812 is not drawn to scale. Tubing 812 can comprise the same or similar features and characteristics as those depicted and described with respect to tubing 116.

In the embodiment shown in FIG. 12, shaft 808 is configured to be coupled to housing 816 and is depicted extending from and coupled to housing 816 in the embodiments shown. In the embodiment shown in FIG. 12, a head can further be coupled to shaft 808 by instrument control system 820 (or can be indirectly coupled to instrument control system 820), at least a portion of which is disposed within housing 816 and at least a portion of which is depicted in FIG. 12. Instrument control system 820 can comprise the same or similar features and characteristics that operate in the same or similar way as those depicted and described with respect to instrument control system 126, including instrument control shafts, control shaft engagement surfaces, pulleys, and wires, some of which are not depicted in FIG. 12. Instrument control system 820 can be configured to enable an operator to control instrument 804, including moving a head with respect to shaft 808 (e.g., such as an operator of a device, including a platform for minimally invasive procedures).

In the embodiment shown in FIG. 12, instrument 804 is configured to enable fluid reservoir 824 and a head of the present disclosure to be in fluid communication. In the embodiment shown, instrument 804 and, more specifically, housing 816 is configured to be coupled to (and decoupled from) fluid reservoir 824 via spike 826, which can be comprised of any suitable rigid material, such as metal, plastic, ceramic, and the like. Spike 826 comprises lumen 827 that is in fluid communication with tubing 812 such that, if fluid reservoir 824 is coupled to spike 826 (e.g., such as by inserting spike 826 into fluid reservoir 824), fluid reservoir 824 is in fluid communication with tubing 812 interior to housing 816. Tubing 812 extends from wall 832 of housing 816, through pump 828, through housing 816, through shaft 808, and into a head to which instrument 804 can be coupled. In the embodiment shown, fluid reservoir 824 is a vial; however, in other embodiments, fluid reservoir 824 can be a syringe, a collapsible bag, and/or any other container configured to accommodate fluid. In some embodiments, tubing 812 can be in fixed connection spike 826 and/or wall 832 of housing 816 and pump 828; and, in other embodiments, tubing 812 can be couplable to and/or decouplable from spike 826 and/or wall 832 of housing 816 and pump 828 by any suitable connection, such as, for example, a screw connection, a snap connection, a Luer taper connection, and similar connections. Tubing 812 can extend from wall 832 of housing 816 to a head of instrument 804 in one or more pieces of tubing. Fluid within fluid reservoir 824 can comprise any fluid described above with respect to fluid reservoir 204.

In some embodiments, if fluid reservoir 824 contains fluid, fluid exits fluid reservoir 824 and can move into housing 816, through pump 828 and, through tubing 812 extending through shaft 808, and into a head to which instrument 804 can be coupled due at least in part to force of gravity. In some embodiments, if fluid reservoir 824 contains fluid, fluid exits fluid reservoir 824 and can move into housing 816, through pump 828, through tubing 812 extending through shaft 808, and into a head to which instrument 804 can be coupled due at least in part to force applied to fluid reservoir 824 by an operator (e.g., by applying manual pressure to fluid reservoir 824, such as by squeezing fluid reservoir 824, to assist fluid in exiting fluid reservoir 824). In the embodiment shown, if fluid reservoir 824 contains fluid, instrument 804 (and, more specifically, pump 828) is further configured to pump and/or encourage fluid from fluid reservoir 824 into housing 816, through tubing 812 extending through shaft 808, and a head to which instrument 804 can be coupled. In the embodiment shown, pump 828 is disposed within to housing 816; however, in other embodiments, pump 828 can be disposed exterior to housing 816 while still being configured to pump fluid from fluid reservoir 824 into housing 816, through tubing 812 extending through shaft 808, and into a head to which instrument 804 can be coupled. In the embodiment shown, pump 828 can be a positive displacement pump, a mechanical pump, an electrical pump, a pneumatic pump, an impulse pump, a gravity pump, and any combination thereof. Pump 828 can further be coupled to a power source (e.g., batteries or an electrical source) that is configured to provide power to pump 828 when activated.

System 800 and/or a device to which instrument 804 is coupled, such as a platform for minimally invasive procedures, can be configured to permit delivery of a predetermined amount of fluid (and/or a substantially equal volume of fluid in succession). For example, if an operator of a platform for minimally invasive procedures to which instrument 804 is coupled activates pump 828, pump 828 can pump a predetermined amount of fluid from fluid reservoir 824 into tubing 812 (and, after such amount of fluid has entered tubing 812, pump 828 can deactivate, either by the operator or automatically).

As discussed above, instrument 804 can comprise any of the head configurations described or depicted in this disclosure that can operate in the same or similar way. In this way, system 800 may be used in minimally invasive procedures. For example, instrument 804 can be coupled to a platform for minimally invasive procedures (similar to that depicted in FIG. 6) in order to be controllable by an operator of the platform for minimally invasive procedures. A head comprising or coupled to a needle can be coupled to shaft 808 such that the head and the needle are in fluid communication with tubing 812 extending through shaft 808 and housing 816 and in fluid communication with fluid reservoir 824.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 804, such as a needle, a head, and a portion of shaft 808 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 804 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 804 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 824 can exit fluid reservoir 824 due at least in part to gravity, force applied to fluid reservoir 824, and/or activation of pump 828 (e.g., via a device, such as a platform for minimally invasive procedures, or via housing 816). Fluid from fluid reservoir 824 exits fluid reservoir 824 through lumen 827 of spike 826, enters housing 816, through pump 828, through shaft 808, a head, and a needle, and, if an operator of the platform for minimally invasive procedures has directed instrument 804 (and, more specifically, a needle) to pierce tissue of a subject, fluid exits the needle into tissue of a subject or a lumen of an organ of the subject.

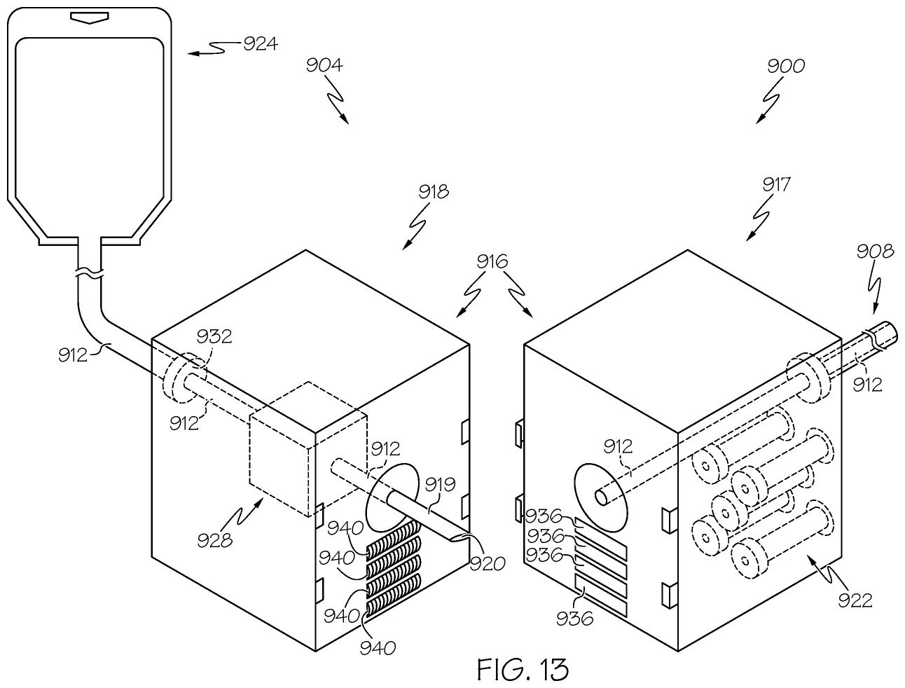

FIG. 13 depicts system 900 comprising instrument 904. In the embodiment shown in FIG. 12, system 900 comprises shaft 908, a portion of which is depicted in FIG. 13, which is configured to be coupled to a head of the present disclosure. Shaft 908 is depicted with two wavy solid lines representing a break in the depiction of shaft 908 to indicate that shaft 908 can extend in the same manner indefinitely between such wavy solid lines and/or can extend at least as long as necessary between such wavy solid lines in order to effect the described embodiments. Therefore, at least some of shaft 908 is not drawn to scale.

The embodiment shown in FIG. 13 further comprises tubing 912, which extends through shaft 908 and toward a head to which instrument 904 can be coupled such that shaft 908 and a head can be in fluid communication. Tubing 912 includes sets of two wavy solid lines depicting breaks in the depiction of tubing 912 to indicate that tubing 912 can extend in the same manner indefinitely between such sets of wavy solid lines and/or can extend at least as long as necessary between such sets of wavy solid lines in order to effect the described embodiments. Therefore, at least some of tubing 912 is not drawn to scale. Tubing 912 can comprise the same or similar features and characteristics as those depicted and described with respect to tubing 116.

In the embodiment shown in FIG. 13, shaft 908 is configured to be coupled to housing 916 and, more specifically, first end 917 of housing 916, and is depicted extending from, coupled to, and in fluid communication with (via tubing 912) first end 917 of housing 916 in the embodiment shown. In the embodiment shown, system 900 further comprises second end 918 of housing 916, which is configured to be coupled to first end 917 of housing 916. In the embodiment shown, second end 918 of housing 916 is configured to be coupled to (and decoupled from) first end 917 of housing 916 via spike 919, which can be comprised of any suitable rigid material, such as metal, plastic, ceramic, and the like. Spike 919 comprises lumen 920 that is in fluid communication with tubing 912 extending through second end 918 of housing 918 such that, if spike 919 is coupled to first end 917 of housing 916 (e.g., such as by inserting spike 919 into tubing 912 of first end 917 of housing 916), lumen 920 of spike 919 is in fluid communication with tubing 912 extending through first end 917 of housing 916 and, therefore tubing 912 of second end 918 of housing 916 is in fluid communication with tubing 912 of first end 917 of housing 916. In other embodiments, first end 917 and second end 918 of housing 916 can be coupled in any suitable way to maintain fluid communication between tubing 912 extending through first end 917 and second end 918 of housing 916 (e.g., such as with a screw connection, a snap connection, a Luer taper, and the like).

In the embodiment shown in FIG. 13, a head can further be coupled to shaft 908 by instrument control system 922 (or can be indirectly coupled to instrument control system 922), at least a portion of which is disposed within first end 917 of housing 916 and at least a portion of which is depicted in FIG. 13. Instrument control system 922 can comprise the same or similar features and characteristics that operate in the same or similar way as those depicted and described with respect to instrument control system 126, including instrument control shafts, control shaft engagement surfaces, pulleys, and wires, some of which are not depicted in FIG. 13. Instrument control system 922 can be configured to enable an operator to control instrument 904, including moving a head with respect to shaft 908 (e.g., such as an operator of a device, including a platform for minimally invasive procedures).

In the embodiment shown in FIG. 13, if first end 917 of housing 916 is coupled to second end 918 of housing 916, instrument 904 is configured to enable fluid reservoir 924 and a head of the present disclosure to be in fluid communication, such as, for example, via tubing 912, which extends from fluid reservoir 924, through pump 928, through second end 918 of housing 916, through spike 919, through first end 917 of housing 916, through shaft 908, and into a head to which instrument 904 can be coupled. In the embodiment shown, fluid reservoir 924 is a collapsible bag (e.g., an intravenous fluid bag). In other embodiments, fluid reservoir 924 can be a syringe, a vial, and/or any other container configured to accommodate fluid. In the embodiment shown, fluid reservoir 924 is exterior to housing 916; however, in other embodiments, fluid reservoir 924 can be within second end 918 of housing 916, within first end 917 of housing 916, or within both first end 917 and second end 918 of housing 916, for example, in any of the ways described and depicted in this disclosure with respect to other embodiments. Tubing 912 is configured to be coupled to (and decoupled from) fluid reservoir 924 (and is depicted coupled to fluid reservoir 924, in the embodiment shown). Tubing 912 enters second end 918 of housing 916 through wall 932 of second end 918 of housing 916. In some embodiments, tubing 912 comprises a fixed connection with wall 932 of second end 918 of housing 516; and in other embodiments, tubing 912 is couplable to and/or decouplable from wall 932 of second end 918 of housing 916. Tubing 912 extends toward and is coupled to pump 928, which is disposed within second end 918 of housing 916. In the embodiment shown, tubing 912 comprises a fixed connection with pump 928 such that tubing 912 cannot be decoupled from pump 928; however, in some embodiments, tubing 912 can be configured to be coupled to (and decoupled from) pump 928. Tubing 912 can be couplable to and/or decouplable from fluid reservoir 924, pump 928, and wall 932 of second end 918 of housing 916 by any suitable connection, such as, for example, a screw connection, a snap connection, a Luer taper connection, a spike connection in which a piercing device is coupled to a portion of tubing 912 and can be pierced through a portion of fluid reservoir 924, pump 928, or wall 932 of second end 918 of housing 916, and similar connections. Tubing 912 can extend from fluid reservoir 924 to a head of instrument 904 in one or more pieces of tubing. Fluid within fluid reservoir 924 can comprise any fluid described above with respect to fluid reservoir 204.

In some embodiments, if fluid reservoir 924 contains fluid, and if first end 917 and second end 918 of housing 916 are coupled, fluid exits fluid reservoir 924 and can move into second end 918 of housing 916, through pump 928, through first end 917 of housing 916, through tubing 912 extending through shaft 908, and into a head to which instrument 904 can be coupled due at least in part to force of gravity. In some embodiments, if fluid reservoir 924 contains fluid, and if first end 917 and second end 918 of housing 916 are coupled, fluid exits fluid reservoir 924 and can move into second end 918 of housing 916, through pump 928, through first end 917 of housing 916, through tubing 912 extending through shaft 908, and into a head to which instrument 904 can be coupled due at least in part to force applied to fluid reservoir 924 by an operator (e.g., by applying manual pressure to fluid reservoir 924, such as by squeezing fluid reservoir 924, to assist fluid in exiting fluid reservoir 924). In the embodiment shown, if fluid reservoir 924 contains fluid, and if first end 917 and second end 918 of housing 916 are coupled, instrument 904 (and, more specifically, pump 928) is further configured to pump and/or encourage fluid from fluid reservoir 924 into second end 918 of housing 916, through first end 917 of housing 916, through tubing 912 extending through shaft 908, and into a head to which instrument 904 can be coupled. In the embodiment shown, pump 928 is disposed within second end 918 of housing 916; however, in other embodiments, pump 928 can be disposed within first end 917 of housing 916 or exterior to housing 916 while still being configured to pump fluid from fluid reservoir 924 into housing 916, through tubing 912 extending through shaft 908, and into a head to which instrument 904 can be coupled. In the embodiment shown, pump 928 can be a positive displacement pump, a mechanical pump, an electrical pump, a pneumatic pump, an impulse pump, a gravity pump, and any combination thereof. Pump 928 can further be coupled to a power source (e.g., batteries or an electrical source) that is configured to provide power to pump 928 when activated.

First end 917 of housing 916 comprises electrical connections 936 and second end 918 of housing 916 comprises electrical connections 940. Electrical connections 936 and 940 are positioned such that, if first end 917 and second end 918 of housing 916 are coupled, electrical connections 936 and 940 are in electrical communication. Components of instrument 904 disposed within first end 917 of housing 916 can therefore communicate electrically with components of instrument 904 disposed within second end 918 of housing 916. For example, in some embodiments, pump 928 is in electrical communication with instrument control system 922 to enable an operator to control pump 928 via instrument control system 922.

System 900 and, more specifically, housing 916 of instrument 904 can be configured to enable some components of system 900 to be disposable and some components of system 900 to be reusable. For example, in the embodiment shown, first end 917 of housing 916 can be reusable, and second end 918 of housing 916 and fluid reservoir 924 can be disposable. In some embodiments, pump 928 and/or fluid reservoir 924 can be disposed within first end 917 of housing 916 such that pump 928 and/or fluid reservoir 924 can be reusable. Reusable components of system 900 can be made from any suitable materials that enable such components to be sterilized. For example, tubing 912 within first end 917 of housing 916 can comprise metal tubing.

System 900 and/or a device to which instrument 904 is coupled, such as a platform for minimally invasive procedures, can be configured to permit delivery of a predetermined amount of fluid (and/or a substantially equal volume of fluid in succession). For example, if first end 917 and second end 918 of housing 916 are coupled, and if an operator of a platform for minimally invasive procedures to which instrument 904 is coupled activates pump 928, pump 928 can pump a predetermined amount of fluid from fluid reservoir 924 into tubing 912 (and, after such amount of fluid has entered tubing 912, pump 928 can deactivate, either by the operator or automatically).

As discussed above, instrument 904 can comprise any of the head configurations described or depicted in this disclosure that can operate in the same or similar way. In this way, system 900 may be used in minimally invasive procedures. For example, instrument 904 can be coupled to a platform for minimally invasive procedures (similar to that depicted in FIG. 6) in order to be controllable by an operator of the platform for minimally invasive procedures. A head comprising or coupled to a needle can be coupled to shaft 908 such that the head and the needle are in fluid communication with tubing 912 extending through shaft 908, in fluid communication with tubing 912 through first end 917 of housing 916, in fluid communication with tubing 912 extending through second end 918 of housing 916, and in fluid communication with fluid reservoir 924.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 904, such as a needle, a head, and a portion of shaft 908 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 904 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 904 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 924 can exit fluid reservoir 924 due at least in part to gravity, force applied to fluid reservoir 924, and/or activation of pump 928 (e.g., via a device, such as a platform for minimally invasive procedures, or via housing 916). Fluid from fluid reservoir 924 exits fluid reservoir 924 through tubing 912, enters second end 918 of housing 916, first end 917 of housing 916, shaft 908, a head, and a needle, and, if an operator of the platform for minimally invasive procedures has directed instrument 904 (and, more specifically, a needle) to pierce tissue of a subject, fluid exits the needle into tissue of a subject or a lumen of an organ of the subject.

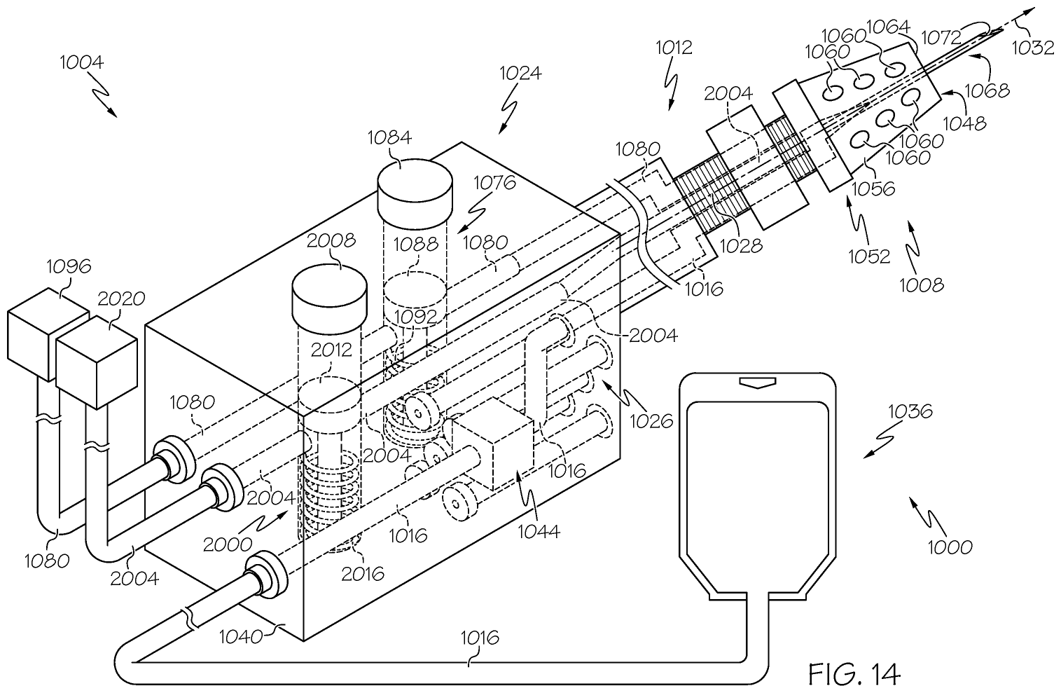

FIGS. 14-18 depict additional examples of the present instruments, systems, methods, and components thereof. As explained above, aspects of any of the embodiments depicted or described above or below may be combined with aspects of the embodiments depicted and described with respect to FIGS. 14-18 to form further embodiments having comparable or different properties and addressing the same or different problems. The embodiments depicted in FIGS. 14-18 and described below can be modified, adapted, and/or supplemented to comprise any of the features shown or described with respect to any of the other systems, instruments, or methods in this disclosure. Similarly, embodiments depicted in other figures or described with respect thereto can be modified, adapted, and/or supplemented to comprise any of the features shown or described with respect to the systems, instruments, and methods of FIGS. 14-18. One or more features of FIGS. 14-18 having the same or similar reference numeral as other features of other embodiments in this disclosure should not be construed to indicate that any feature is limited to the characteristics of another feature having the same or similar reference numeral, or that any feature cannot already have, or cannot be modified to have, features that are different from another feature having the same or similar reference numeral. Though the same or similar feature may have a detailed description with respect to a first embodiment, a second embodiment having the same or similar feature without such a detailed description of such feature (or without any description of such feature) should not be understood to preclude such feature of the second embodiment from comprising the same or similar characteristics as depicted or described with respect to the feature of the first embodiment. For the sake of brevity, all features that an embodiment has or could have may not be described in each separate embodiment.

FIG. 14 depicts system 1000 comprising instrument 1004. In the embodiment shown in FIG. 14, system 1000 comprises head 1008, which is configured to be coupled to shaft 1012 and is depicted coupled to shaft 1012 in the embodiment shown. In FIG. 14, shaft 1012 is depicted with two wavy solid lines representing a break in the depiction of shaft 1012 to indicate that shaft 1012 can extend in the same manner indefinitely between such wavy solid lines and/or can extend at least as long as necessary between such wavy solid lines in order to effect the described embodiments. Therefore, at least some of shaft 1012 is not drawn to scale. Head 1008 can be coupled to shaft 1012 in any suitable way, including in any suitable way to enable movement of head 1008 with respect to shaft 1012, such as, for example, by one or more pins, screws, hinges, bolts, adhesive, and the like, and/or coupled by or formed from the same or similar material as the shaft. In some embodiments, head 1008 is decouplable from shaft 1012 to enable replacement of head 1008 with a head having similar functionality and/or replacement of head 1008 with a head having different functionality. In other embodiments, head 1008 is unitary with shaft 1012 (e.g., at least partially formed of a single piece of material and, therefore, not couplable to or decouplable from shaft 1012).

In the embodiment shown in FIG. 14, head 1008 is further coupled to shaft 1012 by tubing 1016, which extends through a lumen of shaft 1012 and through head 1008 such that shaft 1012 and head 1008 are in fluid communication. Tubing 1016 includes sets of two wavy solid lines depicting breaks in the depiction of tubing 1016 to indicate that tubing 1016 can extend in the same manner indefinitely between each set of such wavy solid lines and/or can extend at least as long as necessary between each set of such wavy solid lines in order to effect the described embodiments. Therefore, at least some of tubing 1016 is not drawn to scale. Tubing 1016 can comprise the same or similar features and characteristics as those depicted and described with respect to tubing 116.

In the embodiment shown in FIG. 14, shaft 1012 is configured to be coupled to housing 1024 and is depicted extending from and coupled to housing 1024 in the embodiment shown. In the embodiment shown in FIG. 14, head 1008 is further coupled to shaft 1012 by instrument control system 1026, at least a portion of which is disposed within housing 1024 and at least a portion of which is depicted in FIG. 14. Instrument control system 1026 can comprise the same or similar features and characteristics that operate in the same or similar way as those depicted and described with respect to instrument control system 126, including instrument control shafts, control shaft engagement surfaces, pulleys, and wires, some of which are not depicted in FIG. 14. Instrument control system 1026 can be configured to enable an operator to control instrument 1004, including moving head 1008 with respect to shaft 1012, as described above (e.g., such as an operator of a device, including a platform for minimally invasive procedures).

In some embodiments, head 1008 is fixed, non-movable, and non-rotatable with respect to shaft 1012. In the embodiment shown in FIG. 14, head 1008 is configured to be movable with respect to shaft 1012. For example, head 1008 and shaft 1012 may be substantially colinear (e.g., a substantial number of points of each of head 1008 and shaft 1012 lie in the same line or linear sequence) and head 1008 is configured to be movable with respect to shaft 1012 such that head 1008 (or at least a portion of head 1008) is non-colinear with respect to shaft 1012 (e.g., at least some points of head 1008 do not lie in the same line or linear sequence as at least some points of shaft 1012). For example, as depicted in FIG. 14, line 1028 is substantially coaxial with shaft 1012, and line 1032 is substantially coaxial with at least a portion of head 1008. In the embodiment shown, lines 1028 and 1032 are substantially colinear and represent an embodiment in which head 1008 and shaft 1012 are substantially colinear. In the embodiment shown, head 1008 is configured to be movable such that lines 1028 and 1032 are substantially non-colinear (and, therefore, represents an embodiment in which head 1008 and shaft 1012 are substantially non-colinear). In some embodiments, head 1008 is configured to be rotatable with respect to shaft 1012 while head 1008 and shaft 1012 remain substantially colinear (e.g., head 1008 may be rotatable 360, 720, 1080 degrees or more while shaft 1012 remains stationary). In such embodiments, head 1008 can comprise at least one degree of freedom with respect to shaft 1012 (e.g., one degree of freedom, two degrees of freedom, three degrees of freedom, four degrees of freedom, five degrees of freedom, and/or six degrees of freedom).

In the embodiment shown in FIG. 14, instrument 1004 is configured to enable fluid reservoir 1036 and head 1008 to be in fluid communication (e.g., via housing 1024 and, more specifically, via tubing 1016 within housing 1024). For example, instrument 1004 is configured such that head 1008 is coupled to and in fluid communication with fluid reservoir 1036 via housing 1024 (and, more specifically, tubing 1016 extending through housing 1024), and shaft 1012 is coupled to and in fluid communication with fluid reservoir 1036 via housing 1024 (and, more specifically, tubing 1016 extending through housing 1024). In the embodiment shown, fluid reservoir 1036 is a collapsible bag (e.g., an intravenous fluid bag). In other embodiments, fluid reservoir 1036 can be a syringe, a vial, and/or any other container configured to accommodate fluid. For example, tubing 1016 extends from head 1008, through a lumen of shaft 1012, through housing 1024, and exits wall 1040 of housing 1024 to enable fluid reservoir 1036 and head 1008 to be in fluid communication via housing 1024. In some embodiments, tubing 1016 comprises a fixed connection with housing 1024 and/or fluid reservoir 1036; and in other embodiments, tubing 1016 is couplable to and/or decouplable from housing 1024 and/or fluid reservoir 1036. Tubing 1016 can be couplable to and/or decouplable from housing 1024 and fluid reservoir 1036 by any suitable connection, such as, for example, a screw connection, a snap connection, a Luer taper connection, a spike connection in which a piercing device is coupled to a portion of tubing 1016 and can be pierced through a portion of fluid reservoir 1036 or housing 1024, and similar connections. In the embodiment shown, fluid from fluid reservoir 1036 can exit fluid reservoir 1036 and move from fluid reservoir 1036, into and through housing 1024, through a lumen of shaft 1012, and into head 1008. Tubing 1016 can extend from fluid reservoir 1036 to head 1008 in one or more pieces of tubing. Fluid within fluid reservoir 1036 can comprise any fluid described above with respect to fluid reservoir 204.

In some embodiments, if fluid reservoir 1036 contains fluid, fluid exits fluid reservoir 1036 and can move into housing 1024, through tubing 1016 extending through a lumen of shaft 1012, and into head 1008 due at least in part to force of gravity. In some embodiments, if fluid reservoir 1036 contains fluid, fluid exits fluid reservoir 1036 and moves into housing 1024, through tubing 1016 extending through a lumen of shaft 1012, and into head 1008 due at least in part to force applied to fluid reservoir 1036 by an operator (e.g., by applying manual pressure to fluid reservoir 1036, such as by squeezing fluid reservoir 1036, to assist fluid in exiting fluid reservoir 1036). In the embodiment shown, if fluid reservoir 1036 contains fluid, instrument 1004 and, more specifically, pump 1044 is configured to pump and/or encourage fluid from fluid reservoir 1036 into housing 1024, through tubing 1016 extending through a lumen of shaft 1012, and into head 1008. In the embodiment shown, pump 1044 is disposed within housing 1024; however, in other embodiments, pump 1044 can be disposed exterior to housing 1024 while still being configured to pump fluid from fluid reservoir 1036 into housing 1024, through tubing 1016 extending through a lumen of shaft 1012, and into head 1008. Pump 1044 can be a positive displacement pump, a mechanical pump, an electrical pump, a pneumatic pump, an impulse pump, a gravity pump, and any combination thereof. Pump 1044 can further be coupled to a power source (e.g., batteries or an electrical source) that is configured to provide power to pump 1044 when activated. System 1000 and/or a device to which instrument 1004 is coupled, such as a platform for minimally invasive procedures, can be configured to permit delivery of a predetermined amount of fluid (and/or a substantially equal volume of fluid in succession), such as through a positive displacement pump, as described above and/or any other pump configured to pump a predetermined amount of fluid, as described herein.

In the embodiment shown in FIG. 14, head 1008 comprises first end 1048, second end 1052, and surface 1056 extending between first end 1048 and second end 1052. Surface 1056 comprises openings 1060, which are configured to enable fluid to enter or exit head 1008. In the embodiment shown, second end 1052 of head 1008 is coupled to shaft 1012. In some embodiments, second end 1052 of head 1008 is couplable to and decouplable from shaft 1012; and in other embodiments, second end 1052 of head 1008 is in fixed connection with shaft 1012 (e.g., such that second end 1052 of head 1008 cannot be decoupled from shaft 1012). First end 1048 of head 1008 comprises end opening 1064, which is configured to enable fluid to enter or exit head 1008. In the embodiment shown, first end 1048 of head 1008 comprises a smaller diameter than second end 1052 of head 1008 and, therefore, surface 1056 extending between first end 1048 and second end 1052 of head 1008 is frustoconically-shaped. In other embodiments, surface 1056 can comprise any suitable shape, such as cylindrical or cubical, to enable fluid to enter or exit head 1008 (e.g., through openings 1060 and/or end opening 1064).

In the embodiment shown, system 1000 further comprises needle 1068 extending from head 1008. More specifically, in the embodiment shown, needle 1068 extends from second end 1052 of head 1008 to first end 1048 of head 1008 and through end opening 1064. Needle 1068 comprises end opening 1072, which is configured to enable fluid to enter and exit needle 1068. In some embodiments, needle 1068 is unitary with head 1008 and/or coupled to and in fixed connection with head 1008 (e.g., such that needle 1068 and head 1008 are at least partially formed of a single piece of material and cannot be decoupled from one another); and, in other embodiments, needle 1068 can be coupled to (or can be configured to be coupled to) and decoupled from head 1008. Needle 1068 is in fluid communication with tubing 1016 extending through head 1008 and, therefore, needle 1068 is in fluid communication with tubing 1016 extending through housing 1024 and with fluid reservoir 1036. In the embodiment shown in FIG. 14, if pump 1044 is activated and if fluid reservoir 1036 comprises fluid, fluid moves from fluid reservoir 1036, into housing 1024, through shaft 1012, into head 1008 and needle 1068, and exits end opening 1072 of needle 1068.

System 1000 further comprises suction system 1076, which is configured to enable fluid and material (e.g., tissue) to be removed from the body of a subject through head 1008. In the embodiment shown in FIG. 14, suction system 1076 comprises tubing 1080, button 1084, stop 1088, and spring 1092. Tubing 1080 includes sets of two wavy solid lines depicting breaks in the depiction of tubing 1080 to indicate that tubing 1080 can extend in the same manner indefinitely between such sets of wavy solid lines and/or can extend at least as long as necessary between such sets of wavy solid lines in order to effect the described embodiments. Therefore, at least some of tubing 1080 is not drawn to scale. Tubing 1080 can comprise the same or similar features and characteristics as those depicted and described with respect to tubing 116. Suction system 1076 can be couplable to and decouplable from suction device 1096 (and is depicted coupled to suction device 1096, in the embodiment shown) via tubing 1080, which exits housing 1024 through wall 1040. In some embodiments, tubing 1080 comprises a fixed connection with housing 1024 and/or suction device 1096; and in other embodiments, tubing 1080 is couplable to and/or decouplable from housing 1024 and/or suction device 1096. Tubing 1080 can be couplable to and/or decouplable from housing 1024 and suction device 1096 by any suitable connection, such as, for example, a screw connection, a snap connection, a Luer taper connection, a spike connection in which a piercing device is coupled to a portion of tubing 1080 and can be pierced through a portion of suction device 1096 or housing 1024, and similar connections. Tubing 1080 extends from suction device 1096, into housing 1024 through wall 1040, through shaft 1012, and into head 1008. Suction device 1096 can be any device configured to apply pressure to tubing 1080 to enable fluid and material near head 1008 to enter head 1008 and move through tubing 1080 toward suction device 1096 (e.g., such as a standalone suction device coupled to a power source, such as batteries or an electrical source, or a suction device structurally integrated with a portion of a room (such as through a wall) in which a medical procedure is being performed).

Suction system 1076 and suction device 1096 are configured to enable system 1000 to be controllable by an operator to enable removal of fluid and material from the body of a subject through head 1008. For example, if instrument 1004 is coupled to a device, such as a platform for minimally invasive procedures, that is configured to enable an operator to control instrument 1004, suction system 1076 and/or suction device 1096 can be coupled to and controllable by such a device to enable an operator of the device to remove fluid and material from the body of a subject through head 1008, such as, for example, by activating suction system 1076 and/or suction device 1096 (and deactivating suction system 1076 and/or suction device 1096 to prevent removal of fluid and material through suction system 1076 and/or suction device 1096). As another example, in the embodiment shown, if button 1084 is engaged by an operator, spring 1092 is depressed such that stop 1088 moves. In some embodiments, stop 1088 can be positioned such that, if button 1084 is unengaged, stop 1088 does not interfere with and/or block tubing 1080 such that fluid is not prevented by stop 1088 from exiting housing 1024 through wall 1040. In such an embodiment, if an operator engages button 1084 to move stop 1088 to interfere with and/or block tubing 1080, fluid and material is prevented from exiting housing 1024 through wall 1040 (in some embodiments, fluid and material is prevented from exiting housing 1024 through wall 1040 even if suction device 1096 is activated). In other embodiments, stop 1088 can be positioned such that, if button 1084 is unengaged, stop 1088 interferes with and/or blocks tubing 1080 such that fluid is prevented by stop 1088 from exiting housing 1024 through wall 1040 (in some embodiments, fluid and material is prevented from exiting housing 1024 through wall 1040 even if suction device 1096 is activated). In such an embodiment, if an operator engages button 1084 to move stop 1088 so that it does not interfere with and/or block tubing 1080, fluid is permitted to exit housing 1024 through wall 1040, such as, for example, if suction device 1096 is activated. In some embodiments, button 1084 can be controllable by or engageable through a platform for minimally invasive procedures. Suction system 1076 and/or suction device 1096 can be configured in various other ways in order to enable removal of fluid and material from the body of a subject by an operator (such as various combinations of pressure sources, pumps, and stop configurations).

System 1000 further comprises irrigation system 2000, which is configured to enable fluid to move into the body of a subject through head 1008 by irrigation system 2000. In the embodiment shown in FIG. 14, irrigation system 2000 comprises tubing 2004, button 2008, stop 2012, and spring 2016. Tubing 2004 includes sets of two wavy solid lines depicting breaks in the depiction of tubing 2004 to indicate that tubing 2004 can extend in the same manner indefinitely between such sets of wavy solid lines and/or can extend at least as long as necessary between such sets of wavy solid lines in order to effect the described embodiments. Therefore, at least some of tubing 2004 is not drawn to scale. Tubing 2004 can comprise the same or similar features and characteristics as those depicted and described with respect to tubing 116. Irrigation system 2000 can be couplable to and decouplable from irrigation device 2020 (and is depicted coupled to irrigation device 2020, in the embodiment shown) via tubing 2004, which exits housing 1024 through wall 1040. In some embodiments, tubing 2004 comprises a fixed connection with housing 1024 and/or irrigation device 2020; and in other embodiments, tubing 2004 is couplable to and/or decouplable from housing 1024 and/or irrigation device 2020. Tubing 2004 can be couplable to and/or decouplable from housing 1024 and irrigation device 2020 by any suitable connection, such as, for example, a screw connection, a snap connection, a Luer taper connection, a spike connection in which a piercing device is coupled to a portion of tubing 2004 and can be pierced through a portion of irrigation device 2020 or housing 1024, and similar connections. Tubing 2004 extends from irrigation device 2020, into housing 1024 through wall 1040, through shaft 1012, and into head 1008. Irrigation device 2020 can be any device configured to enable fluid to move from irrigation device 2020 or from a separate fluid reservoir, through tubing 2004, and into head 1008. For example, in some embodiments irrigation device 2020 can comprise a fluid reservoir, such as a collapsible bag (e.g., an intravenous fluid bag), a syringe, a vial, and/or any other container configured to accommodate fluid. As described above, fluid can be encouraged to exit such a fluid reservoir due at least in part to gravity, force applied to such fluid reservoir, and/or activation of a pump coupled to such fluid reservoir (e.g., via a device, such as a platform for minimally invasive procedures, or via housing 1024). In other embodiments, irrigation device 2020 can comprise a standalone irrigation device (comprising a fluid source and/or configured to be a coupled to a fluid source) coupled to a power source, such as batteries or an electrical source, or an irrigation device structurally integrated with a portion of a room (such as through a wall) in which a medical procedure is being performed.

Irrigation system 2000 and irrigation device 2020 are configured to enable system 1000 to be controllable by an operator to enable fluid to move into the body of a subject through head 1008. For example, if instrument 1004 is coupled to a device, such as a platform for minimally invasive procedures, that is configured to enable an operator to control instrument 1004, irrigation system 2000 and/or irrigation device 2020 can be coupled to and controllable by such a device to enable an operator of the device to move fluid into the body of a subject through head 1008, such as, for example, by activating irrigation system 2000 and/or irrigation device 2020 (and deactivating irrigation system 2000 and/or irrigation device 2020 to prevent fluid from moving into a body of a subject by irrigation system 2000 and/or irrigation device 2020). As another example, in the embodiment shown, if button 2008 is engaged by an operator, spring 2016 is depressed such that stop 2012 moves. In some embodiments, stop 2012 can be positioned such that, if button 2008 is unengaged, stop 2012 does not interfere with and/or block tubing 2004 such that fluid is not prevented by stop 2012 from entering shaft 1012 and head 1008. In such an embodiment, if an operator engages button 2008 to move stop 2012 to interfere with and/or block tubing 2004, fluid is prevented from entering shaft 1012 and head 1008 (in some embodiments, fluid is prevented from entering shaft 1012 and head 1008 even if irrigation device 2020 is activated). In other embodiments, stop 2012 can be positioned such that, if button 2008 is unengaged, stop 2012 interferes with and/or blocks tubing 2004 such that fluid is prevented by stop 2012 from entering shaft 1012 and head 1008 (in some embodiments, fluid is prevented from entering shaft 1012 and head 1008 even if irrigation device 2020 is activated). In such an embodiment, if an operator engages button 2008 to move stop 2012 so that it does not interfere with and/or block tubing 2004, fluid is permitted to enter shaft 1012 and head 1008, such as, for example, if irrigation device 2020 is activated. In some embodiments, button 2008 can be controllable by or engageable through a platform for minimally invasive procedures. Irrigation system 2000 and/or irrigation device 2020 can be configured in various other ways in order to enable movement of fluid into the body of a subject by an operator (such as various combinations of fluid sources, pumps, and stop configurations).

As discussed above, instrument 1004 can comprise any of the head configurations described or depicted in this disclosure that can operate in the same or similar way. In this way, system 1000 may be used in minimally invasive procedures. For example, instrument 1004 can be coupled to a platform for minimally invasive procedures (similar to that depicted in FIG. 6) in order to be controllable by an operator of the platform for minimally invasive procedures. A head comprising or coupled to a needle can be coupled to shaft 1012, such as head 1008 and needle 1068, such that head 1008 and needle 1068 are in fluid communication with tubing 1016 extending through shaft 1012, in fluid communication with tubing 1012 of housing 1024, and in fluid communication with fluid reservoir 1036.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 1004, such as needle 1068, head 1008, and a portion of shaft 1012 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 1004 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 1004 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 1036 can exit fluid reservoir 1036 due at least in part to gravity, force applied to fluid reservoir 1036, and/or activation of pump 1044 (e.g., via a device, such as a platform for minimally invasive procedures, or via housing 1024). Fluid from fluid reservoir 1036 exits fluid reservoir 1036 through tubing 1016, enters housing 1024, shaft 1012, head 1008, and needle 1068, and, if an operator of the platform for minimally invasive procedures has directed instrument 1004 (and, more specifically, needle 1068) to pierce tissue of a subject, fluid exits needle 1068 into tissue of a subject or a lumen of an organ of the subject. Further, in the embodiment shown in FIG. 14, if suction system 1076 and/or suction device 1096 is activated by an operator (e.g., via a device, such as a platform for minimally invasive procedures and/or housing 1024), instrument 1004 is configured to enable an operator to remove fluid and material from the body of a subject (e.g., body 288, as depicted in FIG. 6) through at least one of openings 1060 and/or end opening 1064 of head 1008, through tubing 1080 of head 1008 and shaft 1012, and out of housing 1024 through wall 1040. Additionally, in the embodiment shown in FIG. 14, if irrigation system 2000 and/or irrigation device 2020 is activated by an operator (e.g., via a device, such as a platform for minimally invasive procedures and/or housing 1024), instrument 1004 is configured to enable an operator to move fluid into the body of a subject (e.g., body 288, as depicted in FIG. 6) through tubing 1080 of shaft 1012 and head 1008 and out of head 1008 through at least one of openings 1060 and/or end opening 1064.

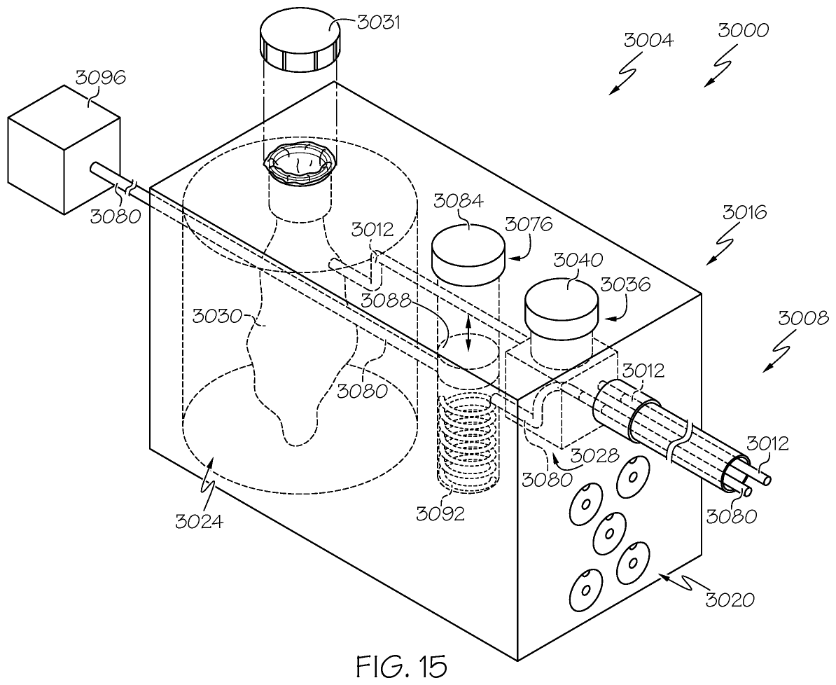

FIGS. 15-18 depict various examples of housings, fluid reservoirs, pumps, irrigation systems and devices, suction systems and devices, configurations thereof, and mechanisms to permit and/or encourage fluid (and sometimes body material) to move through the depicted systems. Though the systems of FIGS. 15-18 may not depict each feature or component depicted or described with respect to other embodiments, such as heads of instruments, needles, instrument control systems or components thereof, and the like, it should be understood that FIGS. 15-18 can comprise any such components that operate in the same or similar manner as described or depicted in this disclosure.

FIG. 15 depicts system 3000 comprising instrument 3004. In the embodiment shown in FIG. 15, system 3000 comprises shaft 3008, a portion of which is depicted in FIG. 13, which is configured to be coupled to a head of the present disclosure. Shaft 3008 is depicted with two wavy solid lines representing a break in the depiction of shaft 3008 to indicate that shaft 3008 can extend in the same manner indefinitely between such wavy solid lines and/or can extend at least as long as necessary between such wavy solid lines in order to effect the described embodiments. Therefore, at least some of shaft 3008 is not drawn to scale.

The embodiment shown in FIG. 15 further comprises tubing 3012, which extends through shaft 3008 and toward a head to which instrument 3004 can be coupled such that shaft 3008 and a head can be in fluid communication. Tubing 3012 includes two wavy solid lines depicting breaks in the depiction of tubing 3012 to indicate that tubing 3012 can extend in the same manner indefinitely between such wavy solid lines and/or can extend at least as long as necessary between such wavy solid lines in order to effect the described embodiments. Therefore, at least some of tubing 3012 is not drawn to scale. Tubing 3012 can comprise the same or similar features and characteristics as those depicted and described with respect to tubing 116.

In the embodiment shown in FIG. 15, shaft 3008 is configured to be coupled to housing 3016 and is depicted extending from and coupled to housing 3016 in the embodiments shown. In the embodiment shown in FIG. 15, a head can further be coupled to shaft 3008 by instrument control system 3020 (or can be indirectly coupled to instrument control system 3020), at least a portion of which is disposed within housing 3016 and at least a portion of which is depicted in FIG. 15. Instrument control system 3020 can comprise the same or similar features and characteristics that operate in the same or similar way as those depicted and described with respect to instrument control system 126, including instrument control shafts, control shaft engagement surfaces, pulleys, and wires, some of which are not depicted in FIG. 15. Instrument control system 3020 can be configured to enable an operator to control instrument 3004, including moving a head with respect to shaft 3008 (e.g., such as an operator of a device, including a platform for minimally invasive procedures).

In the embodiment shown in FIG. 15, instrument 3004 is configured to enable fluid reservoir 3024 and/or collapsible bag 3030 and a head of the present disclosure to be in fluid communication, such as, for example, via tubing 3012, which extends from fluid reservoir 3024 and/or collapsible bag 3030, through housing 3016 and pump 3028, through shaft 3008, and into a head to which instrument 3004 can be coupled. In the embodiment shown, housing 3016 comprises fluid reservoir 3024 into which collapsible bag 3030 can be disposed. In the embodiment shown, fluid reservoir 3024 is cylindrically-shaped; however, fluid reservoir can comprise any suitable shape that is configured to accommodate fluid, including conically-shaped, frustoconically-shaped, cubically-shaped, and the like. Fluid reservoir 3024 is unitary with housing 3016, substantially rigid, and at least partially formed of the same piece of material as housing 3016. In some embodiments, fluid reservoir 3024 can be placed in fluid communication with tubing 3012 to enable fluid from fluid reservoir 3024 to be in fluid communication with shaft 3008 and a head to which instrument 3004 can be coupled. In the embodiment shown, collapsible bag 3030 (e.g., an intravenous fluid bag) acts as fluid reservoir 3024 by being disposed within fluid reservoir 3024 and being couplable to and decouplable from tubing 3012. Tubing 3012 can be couplable to and/or decouplable from fluid reservoir 3024 and/or collapsible bag 3030 by any suitable connection, such as, for example, a screw connection, a snap connection, a Luer taper connection, a spike connection in which a piercing device is coupled to a portion of tubing 3012 and can be pierced through a portion of fluid reservoir 3024 and/or collapsible bag 3030, and similar connections. In this way, collapsible bag 3030 is couplable to and decouplable from housing 3016 and becomes fluid reservoir 3024 to, for example, enable an operator to replace or exchange types of fluid by replacing or exchanging types of collapsible bags. Collapsible bag 3030 can be secured to housing 3016 with cap 3031, which can be coupled to housing 3016 and/or collapsible bag 3030 in any suitable way, such as a threaded connection, a snap connection, and the like. Cap 3031 is configured to prevent fluid from exiting housing 3016 if collapsible bag 3030 is disposed within housing 3016 or if instrument 3004 is configured to operate without collapsible bag 3030 and, instead, fluid reservoir 3024 is coupled to tubing 3012. In some embodiments, at least a portion of cap 3031 can comprise rubber such that cap 3031 can be pierced by a piercing device to enable fluid to be transferred through cap 3031 and into fluid reservoir 3024 and/or collapsible bag 3030. In such an embodiment, fluid reservoir 3024 can comprise fluid from a syringe, a vial, and/or any other container configured to accommodate fluid. Fluid within fluid reservoir 3024 and/or collapsible bag 3030 can comprise any fluid described above with respect to fluid reservoir 204.

In the embodiment shown, tubing 3012 extends from fluid reservoir 3024 and/or collapsible bag 3030 and extends toward pump 3028. In the embodiment shown, tubing 3012 is in fixed connection with pump 3028 and, therefore, cannot be decoupled from pump 3028; however, in other embodiments, tubing 3012 is configured to be coupled to (and decoupled from) pump 3028. Tubing 3012 enters and exits pump 3028 and enters shaft 3008 and continues toward a head to which instrument 3004 can be coupled. Tubing 3012 can extend from fluid reservoir 3024 and/or collapsible bag 3030 to a head of instrument 3004 in one or more pieces of tubing.

In some embodiments, if fluid reservoir 3024 and/or collapsible bag 3030 contains fluid, fluid exits fluid reservoir 3024 and/or collapsible bag 3030 and can move through pump 3028, through tubing 3012 extending through shaft 3008, and into a head to which instrument 3004 can be coupled due at least in part to force of gravity. In some embodiments, if fluid reservoir 3024 and/or collapsible bag 3030 contains fluid, fluid exits fluid reservoir 3024 and/or collapsible bag 3030 and can move through pump 3028, through tubing 3012 extending through shaft 3008, and into a head to which instrument 3004 can be coupled due at least in part to force applied to collapsible bag 3030 by an operator (e.g., by applying manual pressure to collapsible bag 3030, such as by squeezing collapsible bag 3030, to assist fluid in exiting collapsible bag 3030). In the embodiment shown, if fluid reservoir 3024 and/or collapsible bag 3030 contains fluid, instrument 3004 (and, more specifically, pump 3028) is further configured to pump and/or encourage fluid from fluid reservoir 3024 and/or collapsible bag 3030, through tubing 3012 extending through shaft 3008, and into a head to which instrument 3004 can be coupled. In the embodiment shown, pump 3028 is disposed within housing 3016; however, in other embodiments, pump 3028 can be exterior to housing 3016 while still being configured to pump fluid from fluid reservoir 3024 and/or collapsible bag 3030, through tubing 3012 extending through shaft 3008, and into a head to which instrument 3004 can be coupled. In the embodiment shown, pump 3028 can be a positive displacement pump, a mechanical pump, an electrical pump, a pneumatic pump, an impulse pump, a gravity pump, and any combination thereof. Pump 3028 can further be coupled to a power source (e.g., batteries or an electrical source) that is configured to provide power to pump 3028 when activated. In some embodiments, fluid reservoir 3024 is coupled to a pressurizing device such that, if activated, the pressurizing device pressurizes fluid reservoir 3024 to apply a pressure to collapsible bag 3030 in order to encourage fluid from collapsible bag 3030 to exit collapsible bag 3030 into tubing 3012.

In the embodiment shown in FIG. 15, instrument 3004 and, more specifically, housing 3016, further comprises fluid control system 3036 that is configured to enable housing 3016 to be controllable by an operator to enable fluid to enter shaft 3008 and/or to be controllable by an operator to prevent fluid from entering shaft 3008. For example, in the embodiment shown, fluid control system 3036 comprises button 3040 coupled to pump 3028. In some embodiments, if button 3040 is engaged by an operator fluid is permitted to enter shaft 3008. In some embodiments, button 3040 can comprise various engagement positions in which a first engagement position permits a first volume of fluid to enter shaft 3008, a second engagement position permits a second volume of fluid to enter shaft 3008, and so forth. In other embodiments, if button 3040 is engaged, fluid is prevented from entering shaft 3008 (in some embodiments, even if pump 3028 is activated by a device, such as a platform for minimally invasive procedures). In some embodiments, button 3040 can be controllable by or engageable through a platform for minimally invasive procedures. Fluid control system 3036 is configured to enable system 3000 to be controllable by an operator to enable fluid to move into the body of a subject through a head to which instrument 3004 can be is coupled. For example, if instrument 3004 is coupled to a device, such as a platform for minimally invasive procedures, that is configured to enable an operator to control instrument 3004, fluid control system 3036 can be coupled to and controllable by such a device to enable an operator of the device to move fluid into the body of a subject through a head to which instrument 3004 can be coupled, such as, for example, by activating fluid control system 3036 (and deactivating fluid control system 3036 to prevent fluid from moving into a body of a subject by fluid control system 3036). Fluid control system 3036 can be configured in various other ways in order to enable movement of fluid into the body of a subject by an operator (such as various combinations of fluid sources, pumps, and stop configurations).

System 3000 further comprises suction system 3076, which is configured to enable fluid and material (e.g., tissue) to be removed from the body of a subject through a head to which instrument 3004 can be coupled. In the embodiment shown in FIG. 15, suction system 3076 comprises tubing 3080, button 3084, stop 3088, and spring 3092. Tubing 3080 includes sets of two wavy solid lines depicting breaks in the depiction of tubing 3080 to indicate that tubing 3080 can extend in the same manner indefinitely between such sets of wavy solid lines and/or can extend at least as long as necessary between such sets of wavy solid lines in order to effect the described embodiments. Therefore, at least some of tubing 3080 is not drawn to scale. Tubing 3080 can comprise the same or similar features and characteristics as those depicted and described with respect to tubing 116. Suction system 3076 can be couplable to and decouplable from suction device 3096 (and is depicted coupled to suction device 3096, in the embodiment shown) via tubing 3080, which exits housing 3016. In some embodiments, tubing 3080 comprises a fixed connection with housing 3016 and/or suction device 3096; and in other embodiments, tubing 3080 is couplable to and/or decouplable from housing 3016 and/or suction device 3096. Tubing 3080 can be couplable to and/or decouplable from housing 3016 and suction device 3096 by any suitable connection, such as, for example, a screw connection, a snap connection, a Luer taper connection, a spike connection in which a piercing device is coupled to a portion of tubing 3080 and can be pierced through a portion of suction device 3096 or housing 3016, and similar connections. Tubing 3080 extends from suction device 3096, into housing 3016, through shaft 3008, and into a head to which instrument 3004 can be coupled. Suction device 3096 can be any device configured to apply pressure to tubing 3080 to enable fluid and material near a head to which instrument 3004 can be coupled to enter a head to which instrument 3004 can be coupled and move through tubing 3080 toward suction device 3096 (e.g., such as a standalone suction device coupled to a power source, such as batteries or an electrical source, or a suction device structurally integrated with a portion of a room (such as through a wall) in which a medical procedure is being performed).

Suction system 3076 and suction device 3096 are configured to enable system 3000 to be controllable by an operator to enable removal of fluid and material from the body of a subject through a head to which instrument 3004 is coupled. For example, if instrument 3004 is coupled to a device, such as a platform for minimally invasive procedures, that is configured to enable an operator to control instrument 3004, suction system 3076 and/or suction device 3096 can be coupled to and controllable by such a device to enable an operator of the device to remove fluid and material from the body of a subject through a head to which instrument 3004 is coupled, such as, for example, by activating suction system 3076 and/or suction device 3096 (and deactivating suction system 3076 and/or suction device 3096 to prevent removal of fluid and material through suction system 3076 and/or suction device 3096). As another example, in the embodiment shown, if button 3084 is engaged by an operator, spring 3092 is depressed such that stop 3088 moves. In some embodiments, stop 3088 can be positioned such that, if button 3084 is unengaged, stop 3088 does not interfere with and/or block tubing 3080 such that fluid is not prevented by stop 3088 from exiting housing 3016. In such an embodiment, if an operator engages button 3084 to move stop 3088 to interfere with and/or block tubing 3080, fluid and material is prevented from exiting housing 3016 (in some embodiments, fluid and material is prevented from exiting housing 3016 even if suction device 3096 is activated). In other embodiments, stop 3088 can be positioned such that, if button 3084 is unengaged, stop 3088 interferes with and/or blocks tubing 3080 such that fluid is prevented by stop 3088 from exiting housing 3016 (in some embodiments, fluid and material is prevented from exiting housing 3016 even if suction device 3096 is activated). In such an embodiment, if an operator engages button 3084 to move stop 3088 so that it does not interfere with and/or block tubing 3080, fluid is permitted to exit housing 3016, such as, for example, if suction device 3096 is activated. In some embodiments, button 3084 can be controllable by or engageable through a platform for minimally invasive procedures. Suction system 3076 and/or suction device 3096 can be configured in various other ways in order to enable removal of fluid and material from the body of a subject by an operator (such as various combinations of pressure sources, pumps, and stop configurations).

System 3000 and/or a device to which instrument 3004 is coupled, such as a platform for minimally invasive procedures, can be configured to permit delivery of a predetermined amount of fluid (and/or a substantially equal volume of fluid in succession). For example, if an operator of a platform for minimally invasive procedures to which instrument 3004 is coupled activates pump 3028, pump 3028 can pump a predetermined amount of fluid from fluid reservoir 3024 and/or collapsible bag 3030 into tubing 3012 (and, after such amount of fluid has entered tubing 3012, pump 3028 can deactivate, either by the operator or automatically). As another example, if an operator engages button 3040, a predetermined amount of fluid can be permitted to move through tubing 3012 past fluid control system 3036 and into shaft 3008 (and, after such amount of fluid has entered shaft 3008, fluid control system 3036 can be configured to prevent additional fluid from moving past fluid control system 3036). In some embodiments, fluid control system 3036 and/or a device, such as a platform for minimally invasive procedures, to which instrument 3004 is coupled can comprise various engagement positions in which a first engagement position permits a first volume of fluid to enter shaft 3008, a second engagement position permits a second volume of fluid to enter shaft 3008, and so forth. As an example, in such an embodiment, a first engagement position may permit a first volume of fluid to enter shaft 3008 to deliver fluid through tissue of a subject, and a second engagement position may permit a second volume of fluid to enter shaft 3008 to irrigate a target area within the body of a subject. Fluid control system 3036 and/or a device, such as a platform for minimally invasive procedures, to which instrument 3004 is coupled can comprise any number of engagement positions that correspond to any number of fluid volumes to, for example, effect a given surgical procedure and/or to effect a desired task.

As discussed above, instrument 3004 can comprise any of the head configurations described or depicted in this disclosure that can operate in the same or similar way. In this way, system 3000 may be used in minimally invasive procedures. For example, instrument 3004 can be coupled to a platform for minimally invasive procedures (similar to that depicted in FIG. 6) in order to be controllable by an operator of the platform for minimally invasive procedures. A head comprising or coupled to a needle can be coupled to shaft 3008 such that the head and the needle are in fluid communication with tubing 3012 extending through shaft 3008 and housing 3016 and in fluid communication with fluid reservoir 3024 and/or collapsible bag 3030.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 3004, such as a needle, a head, and a portion of shaft 3008 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 3004 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 3004 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 3024 and/or collapsible bag 3030 can exit fluid reservoir 3024 and/or collapsible bag 3030 due at least in part to gravity, force applied to collapsible bag 3030, and/or activation of pump 3028 (e.g., via a device, such as a platform for minimally invasive procedures, or via housing 3016). Fluid from fluid reservoir 3024 and/or collapsible bag 3030 exits fluid reservoir 3024 and/or collapsible bag 3030 through tubing 3012, through fluid control system 3036 and pump 3028, through shaft 3008, through a head and a needle to which instrument 3004 is coupled, and, if an operator of the platform for minimally invasive procedures has directed instrument 3004 (and, more specifically, a needle) to pierce tissue of a subject, fluid exits the needle into tissue of a subject or a lumen of an organ of the subject. Additionally, in the embodiment shown in FIG. 15, if suction system 3076 and/or suction device 3096 is activated by an operator (e.g., via a device, such as a platform for minimally invasive procedures and/or housing 3016), instrument 3004 is configured to enable an operator to remove fluid and material from the body of a subject (e.g., body 288, as depicted in FIG. 6) through a head to which instrument 3004 is coupled, through tubing 3080 of shaft 3008, and out of housing 3016.

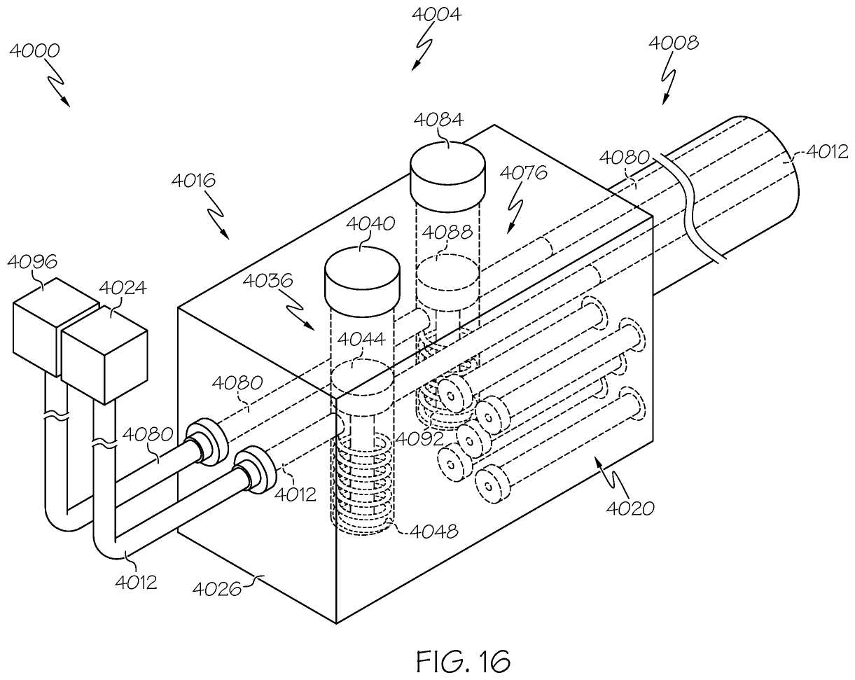

FIG. 16 depicts system 4000 comprising instrument 4004. In the embodiment shown in FIG. 16, system 4000 comprises shaft 4008, a portion of which is depicted in FIG. 16, which is configured to be coupled to a head of the present disclosure. Shaft 4008 is depicted with two wavy solid lines representing a break in the depiction of shaft 4008 to indicate that shaft 4008 can extend in the same manner indefinitely between such wavy solid lines and/or can extend at least as long as necessary between such wavy solid lines in order to effect the described embodiments. Therefore, at least some of shaft 4008 is not drawn to scale.

The embodiment shown in FIG. 16 further comprises tubing 4012, which extends through shaft 4008 and toward a head to which instrument 4004 can be coupled such that shaft 4008 and a head can be in fluid communication. Tubing 4012 includes sets of two wavy solid lines depicting breaks in the depiction of tubing 4012 to indicate that tubing 4012 can extend in the same manner indefinitely between such sets of wavy solid lines and/or can extend at least as long as necessary between such sets of wavy solid lines in order to effect the described embodiments. Therefore, at least some of tubing 4012 is not drawn to scale. Tubing 4012 can comprise the same or similar features and characteristics as those depicted and described with respect to tubing 116.

In the embodiment shown in FIG. 16, shaft 4008 is configured to be coupled to housing 4016 and is depicted extending from and coupled to housing 4016 in the embodiments shown. In the embodiment shown in FIG. 16, a head can further be coupled to shaft 4008 by instrument control system 4020 (or can be indirectly coupled to instrument control system 4020), at least a portion of which is disposed within housing 4016 and at least a portion of which is depicted in FIG. 16. Instrument control system 4020 can comprise the same or similar features and characteristics that operate in the same or similar way as those depicted and described with respect to instrument control system 126, including instrument control shafts, control shaft engagement surfaces, pulleys, and wires, some of which are not depicted in FIG. 16. Instrument control system 4020 can be configured to enable an operator to control instrument 4004, including moving a head with respect to shaft 4008 (e.g., such as an operator of a device, including a platform for minimally invasive procedures).

In the embodiment shown in FIG. 16, instrument 4004 is configured to enable fluid reservoir 4024 and a head of the present disclosure to be in fluid communication, such as, for example, via tubing 4012, which extends from fluid reservoir 4024, through housing 4016, through shaft 4008, and into a head to which instrument 4004 can be coupled. In the embodiment shown, fluid reservoir 4024 comprises any container configured to accommodate fluid, including a collapsible bag (e.g., an intravenous fluid bag), a syringe, a vial, and the like. For example, tubing 4012 extends from a head to which instrument 4004 can be coupled, through a lumen of shaft 4008, through housing 4016, and exits wall 4026 of housing 4016 to enable fluid reservoir 4024 and a head to which instrument 4004 can be coupled to be in fluid communication via housing 4016. In some embodiments, tubing 4012 comprises a fixed connection with housing 4016 and/or fluid reservoir 4024; and in other embodiments, tubing 4012 is couplable to and/or decouplable from housing 4016 and/or fluid reservoir 4024. Tubing 4012 can be couplable to and/or decouplable from housing 4016 and fluid reservoir 4024 by any suitable connection, such as, for example, a screw connection, a snap connection, a Luer taper connection, a spike connection in which a piercing device is coupled to a portion of tubing 4012 and can be pierced through a portion of fluid reservoir 4024 or housing 4016, and similar connections. In the embodiment shown, fluid from fluid reservoir 4024 can exit fluid reservoir 4024 and move from fluid reservoir 4024, into and through housing 4016, through a lumen of shaft 4008, and into a head to which instrument 4004 can be coupled. Tubing 4012 can extend from fluid reservoir 4024 to a head to which instrument 4004 can be coupled in one or more pieces of tubing. Fluid within fluid reservoir 4024 can comprise any fluid described above with respect to fluid reservoir 204.

In the embodiment shown, if fluid reservoir 4024 contains fluid, fluid exits fluid reservoir 4024 and can move into housing 4016 via wall 4026, through tubing 4012 extending through housing 4016 and shaft 4008, and into a head to which instrument 4004 can be coupled due at least in part to force of gravity. In the embodiment shown, if fluid reservoir 4024 contains fluid, fluid exits fluid reservoir 4024 and can move into housing 4016 via wall 4026, through tubing 4012 extending through housing 4016 and shaft 4008, and into a head to which instrument 4004 can be coupled due at least in part to force applied to fluid reservoir 4024 by an operator (e.g., by applying manual pressure to fluid reservoir 4024, such as by squeezing fluid reservoir 4024, to assist fluid in exiting fluid reservoir 4024).

In other embodiments, system 4000 can comprise a pump to pump and/or encourage fluid from fluid reservoir 4024 toward a head to which instrument 4004 can be coupled. Such a pump can be disposed exterior to housing 4016 or interior to housing 4016. Such a pump can be couplable to and decouplable from components of system 4000 or in fixed connection with components of system 4000. In such an embodiment, a pump can be a positive displacement pump, a mechanical pump, an electrical pump, a pneumatic pump, an impulse pump, a gravity pump, and any combination thereof. The pump can further be coupled to a power source (e.g., batteries or an electrical source) that is configured to provide power to the pump when activated.

In the embodiment shown in FIG. 16, instrument 4004 and, more specifically, housing 4016, further comprises fluid control system 4036 that is configured to enable housing 4016 to be controllable by an operator to enable fluid to enter shaft 4008 and/or to be controllable by an operator to prevent fluid from entering shaft 4008. For example, in the embodiment shown, fluid control system 4036 comprises button 4040, stop 4044, and spring 4048 coupled to stop 4044. If button 4040 is engaged by an operator, spring 4048 is depressed such that stop 4044 moves. In some embodiments, stop 4044 can be positioned such that, if button 4040 is unengaged, stop 4044 does not interfere with and/or block tubing 4012 such that fluid is not prevented by stop 4044 from entering shaft 4008. In such an embodiment, if an operator engages button 4040 to move stop 4044 to interfere with and/or block tubing 4012, fluid is prevented from entering shaft 4008. In other embodiments, stop 4044 can be positioned such that, if button 4040 is unengaged, stop 4044 interferes with and/or blocks tubing 4012 such that fluid is prevented by stop 4044 from entering shaft 4008. In such an embodiment, if an operator engages button 4040 to move stop 4044 so that it does not interfere with and/or block tubing 4012, fluid is permitted to move into shaft 4008 (e.g., due to a pressure applied on fluid within tubing 4012 via gravity and/or an applied pressure on fluid reservoir 4024). In some embodiments, button 4040 can be controllable by or engageable through a platform for minimally invasive procedures. In some embodiments, button 4040 can comprise various engagement positions in which a first engagement position permits a first volume of fluid to enter shaft 4008, a second engagement position permits a second volume of fluid to enter shaft 4008, and so forth.

Fluid control system 4036 is configured to enable system 4000 to be controllable by an operator to enable fluid to move into the body of a subject through a head to which instrument 4004 can be is coupled. For example, if instrument 4004 is coupled to a device, such as a platform for minimally invasive procedures, that is configured to enable an operator to control instrument 4004, fluid control system 4036 can be coupled to and controllable by such a device to enable an operator of the device to move fluid into the body of a subject through a head to which instrument 4004 can be coupled, such as, for example, by activating fluid control system 4036 (and deactivating fluid control system 4036 to prevent fluid from moving into a body of a subject by fluid control system 4036). Fluid control system 4036 can be configured in various other ways in order to enable movement of fluid into the body of a subject by an operator (such as various combinations of fluid sources, pumps, and stop configurations).

System 4000 further comprises suction system 4076, which is configured to enable fluid and material (e.g., tissue) to be removed from the body of a subject through a head to which instrument 4004 can be coupled. In the embodiment shown in FIG. 16, suction system 4076 comprises tubing 4080, button 4084, stop 4088, and spring 4092. Tubing 4080 includes sets of two wavy solid lines depicting breaks in the depiction of tubing 4080 to indicate that tubing 4080 can extend in the same manner indefinitely between such sets of wavy solid lines and/or can extend at least as long as necessary between such sets of wavy solid lines in order to effect the described embodiments. Therefore, at least some of tubing 4080 is not drawn to scale. Tubing 4080 can comprise the same or similar features and characteristics as those depicted and described with respect to tubing 116. Suction system 4076 can be couplable to and decouplable from suction device 4096 (and is depicted coupled to suction device 4096, in the embodiment shown) via tubing 4080, which exits housing 4016. In some embodiments, tubing 4080 comprises a fixed connection with housing 4016 and/or suction device 4096; and in other embodiments, tubing 4080 is couplable to and/or decouplable from housing 4016 and/or suction device 4096. Tubing 4080 can be couplable to and/or decouplable from housing 4016 and suction device 4096 by any suitable connection, such as, for example, a screw connection, a snap connection, a Luer taper connection, a spike connection in which a piercing device is coupled to a portion of tubing 4080 and can be pierced through a portion of suction device 4096 or housing 4016, and similar connections. Tubing 4080 extends from suction device 4096, into housing 4016, through shaft 4008, and into a head to which instrument 4004 can be coupled. Suction device 4096 can be any device configured to apply pressure to tubing 4080 to enable fluid and material near a head to which instrument 4004 can be coupled to enter a head to which instrument 4004 can be coupled and move through tubing 4080 toward suction device 4096 (e.g., such as a standalone suction device coupled to a power source, such as batteries or an electrical source, or a suction device structurally integrated with a portion of a room (such as through a wall) in which a medical procedure is being performed).

Suction system 4076 and suction device 4096 are configured to enable system 4000 to be controllable by an operator to enable removal of fluid and material from the body of a subject through a head to which instrument 4004 is coupled. For example, if instrument 4004 is coupled to a device, such as a platform for minimally invasive procedures, that is configured to enable an operator to control instrument 4004, suction system 4076 and/or suction device 4096 can be coupled to and controllable by such a device to enable an operator of the device to remove fluid and material from the body of a subject through a head to which instrument 4004 is coupled, such as, for example, by activating suction system 4076 and/or suction device 4096 (and deactivating suction system 4076 and/or suction device 4096 to prevent removal of fluid and material through suction system 4076 and/or suction device 4096). As another example, in the embodiment shown, if button 4084 is engaged by an operator, spring 4092 is depressed such that stop 4088 moves. In some embodiments, stop 4088 can be positioned such that, if button 4084 is unengaged, stop 4088 does not interfere with and/or block tubing 4080 such that fluid is not prevented by stop 4088 from exiting housing 4016. In such an embodiment, if an operator engages button 4084 to move stop 4088 to interfere with and/or block tubing 4080, fluid and material is prevented from exiting housing 4016 (in some embodiments, fluid and material is prevented from exiting housing 4016 even if suction device 4096 is activated). In other embodiments, stop 4088 can be positioned such that, if button 4084 is unengaged, stop 4088 interferes with and/or blocks tubing 4080 such that fluid is prevented by stop 4088 from exiting housing 4016 (in some embodiments, fluid and material is prevented from exiting housing 4016 even if suction device 4096 is activated). In such an embodiment, if an operator engages button 4084 to move stop 4088 so that it does not interfere with and/or block tubing 4080, fluid is permitted to exit housing 4016, such as, for example, if suction device 4096 is activated. Suction system 4076 and/or suction device 4096 can be configured in various other ways in order to enable removal of fluid and material from the body of a subject by an operator (such as various combinations of pressure sources, pumps, and stop configurations).

System 4000 and/or a device to which instrument 4004 is coupled, such as a platform for minimally invasive procedures, can be configured to permit delivery of a predetermined amount of fluid (and/or a substantially equal volume of fluid in succession). For example, if an operator engages button 4040, a predetermined amount of fluid can be permitted to move through tubing 4012 past fluid control system 4036 and into shaft 4008 (and, after such amount of fluid has entered shaft 4008, fluid control system 4036 can be configured to prevent additional fluid from moving past fluid control system 4036). In some embodiments, fluid control system 4036 and/or a device, such as a platform for minimally invasive procedures, to which instrument 4004 is coupled can comprise various engagement positions in which a first engagement position permits a first volume of fluid to enter shaft 4008, a second engagement position permits a second volume of fluid to enter shaft 4008, and so forth. As an example, in such an embodiment, a first engagement position may permit a first volume of fluid to enter shaft 4008 to deliver fluid through tissue of a subject, and a second engagement position may permit a second volume of fluid to enter shaft 4008 to irrigate a target area within the body of a subject. Fluid control system 4036 and/or a device, such as a platform for minimally invasive procedures, to which instrument 4004 is coupled can comprise any number of engagement positions that correspond to any number of fluid volumes to, for example, effect a given surgical procedure and/or to effect a desired task.

As discussed above, instrument 4004 can comprise any of the head configurations described or depicted in this disclosure that can operate in the same or similar way. In this way, system 4000 may be used in minimally invasive procedures. For example, instrument 4004 can be coupled to a platform for minimally invasive procedures (similar to that depicted in FIG. 6) in order to be controllable by an operator of the platform for minimally invasive procedures. A head comprising or coupled to a needle can be coupled to shaft 4008 such that the head and the needle are in fluid communication with tubing 4012 extending through shaft 4008 and housing 4016 and in fluid communication with fluid reservoir 4024.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 4004, such as a needle, a head, and a portion of shaft 4008 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 4004 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 4004 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 4024 can exit fluid reservoir 4024 due at least in part to gravity, force applied to fluid reservoir 4024, and/or activation of a pump to which system 4000 can be coupled (e.g., via a device, such as a platform for minimally invasive procedures, or via housing 4016). Fluid from fluid reservoir 4024 exits fluid reservoir 4024 through tubing 4012, enters housing 4016 through wall 4026, moves through fluid control system 4036, through shaft 4008, through a head and a needle to which instrument 4004 is coupled, and, if an operator of the platform for minimally invasive procedures has directed instrument 4004 (and, more specifically, a needle) to pierce tissue of a subject, fluid exits the needle into tissue of a subject or a lumen of an organ of the subject. Additionally, in the embodiment shown in FIG. 16, if suction system 4076 and/or suction device 4096 is activated by an operator (e.g., via a device, such as a platform for minimally invasive procedures and/or housing 4016), instrument 4004 is configured to enable an operator to remove fluid and material from the body of a subject (e.g., body 288, as depicted in FIG. 6) through a head to which instrument 4004 is coupled, through tubing 4080 of shaft 4008, and out of housing 4016.

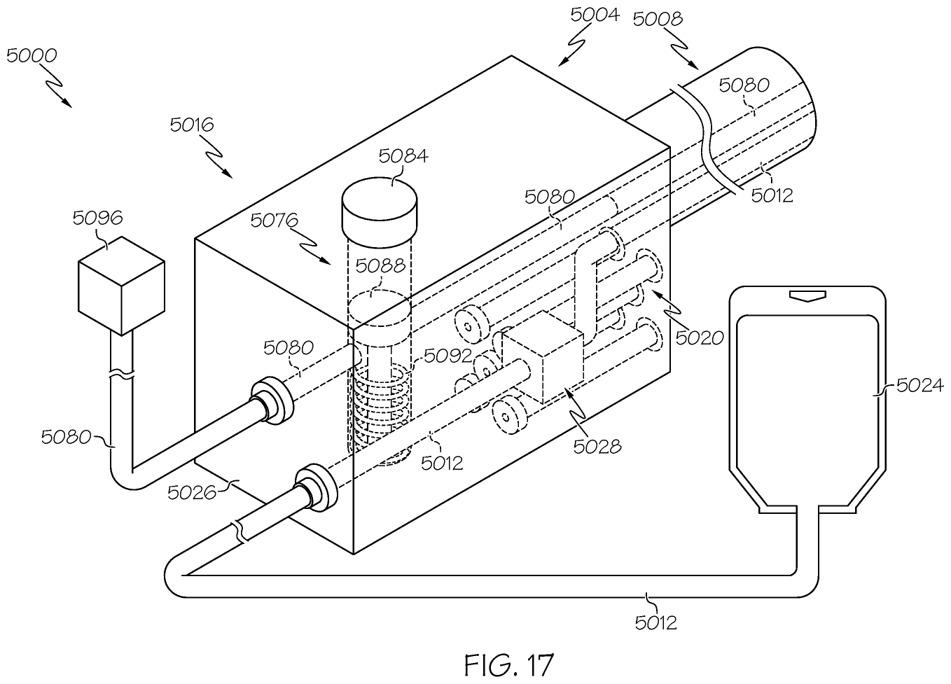

FIG. 17 depicts system 5000 comprising instrument 5004. In the embodiment shown in FIG. 17, system 5000 comprises shaft 5008, a portion of which is depicted in FIG. 17, which is configured to be coupled to a head of the present disclosure. Shaft 5008 is depicted with two wavy solid lines representing a break in the depiction of shaft 5008 to indicate that shaft 5008 can extend in the same manner indefinitely between such wavy solid lines and/or can extend at least as long as necessary between such wavy solid lines in order to effect the described embodiments. Therefore, at least some of shaft 5008 is not drawn to scale.

The embodiment shown in FIG. 17 further comprises tubing 5012, which extends through shaft 5008 and toward a head to which instrument 5004 can be coupled such that shaft 5008 and a head can be in fluid communication. Tubing 5012 includes sets of two wavy solid lines depicting breaks in the depiction of tubing 5012 to indicate that tubing 5012 can extend in the same manner indefinitely between such sets of wavy solid lines and/or can extend at least as long as necessary between such sets of wavy solid lines in order to effect the described embodiments. Therefore, at least some of tubing 5012 is not drawn to scale. Tubing 5012 can comprise the same or similar features and characteristics as those depicted and described with respect to tubing 116.

In the embodiment shown in FIG. 17, shaft 5008 is configured to be coupled to housing 5016 and is depicted extending from and coupled to housing 5016 in the embodiments shown. In the embodiment shown in FIG. 17, a head can further be coupled to shaft 5008 by instrument control system 5020 (or can be indirectly coupled to instrument control system 5020), at least a portion of which is disposed within housing 5016 and at least a portion of which is depicted in FIG. 17. Instrument control system 5020 can comprise the same or similar features and characteristics that operate in the same or similar way as those depicted and described with respect to instrument control system 126, including instrument control shafts, control shaft engagement surfaces, pulleys, and wires, some of which are not depicted in FIG. 17. Instrument control system 5020 can be configured to enable an operator to control instrument 5004, including moving a head with respect to shaft 5008 (e.g., such as an operator of a device, including a platform for minimally invasive procedures).

In the embodiment shown in FIG. 17, instrument 5004 is configured to enable fluid reservoir 5024 and a head of the present disclosure to be in fluid communication, such as, for example, via tubing 5012, which extends from fluid reservoir 5024, through housing 5016, through shaft 5008, and into a head to which instrument 5004 can be coupled. In the embodiment shown, fluid reservoir 5024 is a collapsible bag (e.g., an intravenous fluid bag); however, in other embodiments, fluid reservoir 5024 can comprise a syringe, a vial, and/or any other container configured to accommodate fluid. For example, tubing 5012 extends from a head to which instrument 5004 can be coupled, through a lumen of shaft 5008, through housing 5016, and exits wall 5026 of housing 5016 to enable fluid reservoir 5024 and a head to which instrument 5004 can be coupled to be in fluid communication via housing 5016. In some embodiments, tubing 5012 comprises a fixed connection with housing 5016 and/or fluid reservoir 5024; and in other embodiments, tubing 5012 is couplable to and/or decouplable from housing 5016 and/or fluid reservoir 5024. Tubing 5012 can be couplable to and/or decouplable from housing 5016 and fluid reservoir 5024 by any suitable connection, such as, for example, a screw connection, a snap connection, a Luer taper connection, a spike connection in which a piercing device is coupled to a portion of tubing 5012 and can be pierced through a portion of fluid reservoir 5024 or housing 5016, and similar connections. In the embodiment shown, fluid from fluid reservoir 5024 can exit fluid reservoir 5024 and move from fluid reservoir 5024, into and through housing 5016, through a lumen of shaft 5008, and into a head to which instrument 5004 can be coupled. Tubing 5012 can extend from fluid reservoir 5024 to a head to which instrument 5004 can be coupled in one or more pieces of tubing. Fluid within fluid reservoir 5024 can comprise any fluid described above with respect to fluid reservoir 204.

In the embodiment shown, if fluid reservoir 5024 contains fluid, fluid exits fluid reservoir 5024 and can move into housing 5016 via wall 5026, through tubing 5012 extending through housing 5016 and shaft 5008, and into a head to which instrument 5004 can be coupled due at least in part to force of gravity. In the embodiment shown, if fluid reservoir 5024 contains fluid, fluid exits fluid reservoir 5024 and can move into housing 5016 via wall 5026, through tubing 5012 extending through housing 5016 and shaft 5008, and into a head to which instrument 5004 can be coupled due at least in part to force applied to fluid reservoir 5024 by an operator (e.g., by applying manual pressure to fluid reservoir 5024, such as by squeezing fluid reservoir 5024, to assist fluid in exiting fluid reservoir 5024). In the embodiment shown, if fluid reservoir 5024 contains fluid, instrument 5004 and, more specifically, pump 5028 is configured to pump and/or encourage fluid from fluid reservoir 5024 into housing 5016, through tubing 5012 extending through a lumen of shaft 5008, and into a head to which instrument 5004 can be coupled. In the embodiment shown, pump 5028 is disposed within housing 5016; however, in other embodiments, pump 5028 can be disposed exterior to housing 5016 while still being configured to pump fluid from fluid reservoir 5024 into housing 5016, through tubing 5012 extending through a lumen of shaft 5008, and into a head to which instrument 5004 can be coupled. Pump 5044 can be a positive displacement pump, a mechanical pump, an electrical pump, a pneumatic pump, an impulse pump, a gravity pump, and any combination thereof. Pump 5044 can further be coupled to a power source (e.g., batteries or an electrical source) that is configured to provide power to pump 5044 when activated. System 5000 and/or a device to which instrument 5004 is coupled, such as a platform for minimally invasive procedures, can be configured to permit delivery of a predetermined amount of fluid (and/or a substantially equal volume of fluid in succession), such as through a positive displacement pump, as described above and/or any other pump configured to pump a predetermined amount of fluid, as described herein.

Pump 5028 is configured to enable system 5000 to be controllable by an operator to enable fluid to move into the body of a subject through a head to which instrument 5004 can be is coupled. For example, if instrument 5004 is coupled to a device, such as a platform for minimally invasive procedures, that is configured to enable an operator to control instrument 5004, pump 5028 can be coupled to and controllable by such a device to enable an operator of the device to move fluid into the body of a subject through a head to which instrument 5004 can be coupled, such as, for example, by activating pump 5028 (and deactivating pump 5028 to prevent fluid from moving into a body of a subject by pump 5028). In some embodiments, housing 5016 can enable an operator to activate pump 5028, such as by engaging a button on housing 5016 that is coupled to pump 5028 and enables activation and/or deactivation of pump 5028. System 5000 can be configured in various other ways in order to enable movement of fluid into the body of a subject by an operator (such as various combinations of fluid sources, pumps, and stop configurations).

System 5000 further comprises suction system 5076, which is configured to enable fluid and material (e.g., tissue) to be removed from the body of a subject through a head to which instrument 5004 can be coupled. In the embodiment shown in FIG. 17, suction system 5076 comprises tubing 5080, button 5084, stop 5088, and spring 5092. Tubing 5080 includes sets of two wavy solid lines depicting breaks in the depiction of tubing 5080 to indicate that tubing 5080 can extend in the same manner indefinitely between such sets of wavy solid lines and/or can extend at least as long as necessary between such sets of wavy solid lines in order to effect the described embodiments. Therefore, at least some of tubing 5080 is not drawn to scale. Tubing 5080 can comprise the same or similar features and characteristics as those depicted and described with respect to tubing 116. Suction system 5076 can be couplable to and decouplable from suction device 5096 (and is depicted coupled to suction device 5096, in the embodiment shown) via tubing 5080, which exits housing 5016. In some embodiments, tubing 5080 comprises a fixed connection with housing 5016 and/or suction device 5096; and in other embodiments, tubing 5080 is couplable to and/or decouplable from housing 5016 and/or suction device 5096. Tubing 5080 can be couplable to and/or decouplable from housing 5016 and suction device 5096 by any suitable connection, such as, for example, a screw connection, a snap connection, a Luer taper connection, a spike connection in which a piercing device is coupled to a portion of tubing 5080 and can be pierced through a portion of suction device 5096 or housing 5016, and similar connections. Tubing 5080 extends from suction device 5096, into housing 5016, through shaft 5008, and into a head to which instrument 5004 can be coupled. Suction device 5096 can be any device configured to apply pressure to tubing 5080 to enable fluid and material near a head to which instrument 5004 can be coupled to enter a head to which instrument 5004 can be coupled and move through tubing 5080 toward suction device 5096 (e.g., such as a standalone suction device coupled to a power source, such as batteries or an electrical source, or a suction device structurally integrated with a portion of a room (such as through a wall) in which a medical procedure is being performed).

Suction system 5076 and suction device 5096 are configured to enable system 5000 to be controllable by an operator to enable removal of fluid and material from the body of a subject through a head to which instrument 5004 is coupled. For example, if instrument 5004 is coupled to a device, such as a platform for minimally invasive procedures, that is configured to enable an operator to control instrument 5004, suction system 5076 and/or suction device 5096 can be coupled to and controllable by such a device to enable an operator of the device to remove fluid and material from the body of a subject through a head to which instrument 5004 is coupled, such as, for example, by activating suction system 5076 and/or suction device 5096 (and deactivating suction system 5076 and/or suction device 5096 to prevent removal of fluid and material through suction system 5076 and/or suction device 5096). As another example, in the embodiment shown, if button 5084 is engaged by an operator, spring 5092 is depressed such that stop 5088 moves. In some embodiments, stop 5088 can be positioned such that, if button 5084 is unengaged, stop 5088 does not interfere with and/or block tubing 5080 such that fluid is not prevented by stop 5088 from exiting housing 5016. In such an embodiment, if an operator engages button 5084 to move stop 5088 to interfere with and/or block tubing 5080, fluid and material is prevented from exiting housing 5016 (in some embodiments, fluid and material is prevented from exiting housing 5016 even if suction device 5096 is activated). In other embodiments, stop 5088 can be positioned such that, if button 5084 is unengaged, stop 5088 interferes with and/or blocks tubing 5080 such that fluid is prevented by stop 5088 from exiting housing 5016 (in some embodiments, fluid and material is prevented from exiting housing 5016 even if suction device 5096 is activated). In such an embodiment, if an operator engages button 5084 to move stop 5088 so that it does not interfere with and/or block tubing 5080, fluid is permitted to exit housing 5016, such as, for example, if suction device 5096 is activated. In some embodiments, button 5084 can be controllable by or engageable through a platform for minimally invasive procedures. Suction system 5076 and/or suction device 5096 can be configured in various other ways in order to enable removal of fluid and material from the body of a subject by an operator (such as various combinations of pressure sources, pumps, and stop configurations).

System 5000 and/or a device to which instrument 5004 is coupled, such as a platform for minimally invasive procedures, can be configured to permit delivery of a predetermined amount of fluid (and/or a substantially equal volume of fluid in succession). For example, if an operator activates pump 5028, a predetermined amount of fluid can be permitted to move through tubing 5012 and into shaft 5008 (and, after such amount of fluid has entered shaft 5008, pump 5028 can be configured to prevent additional fluid from moving past pump 5028). In some embodiments, pump 5028 (and, more specifically, a portion of housing 5016 that enables an operator to control pump 5028) and/or a device, such as a platform for minimally invasive procedures, to which instrument 5004 is coupled can comprise various engagement positions in which a first engagement position permits a first volume of fluid to enter shaft 5008, a second engagement position permits a second volume of fluid to enter shaft 5008, and so forth. As an example, in such an embodiment, a first engagement position may permit a first volume of fluid to enter shaft 5008 to deliver fluid through tissue of a subject, and a second engagement position may permit a second volume of fluid to enter shaft 5008 to irrigate a target area within the body of a subject. Pump 5028 and/or a device, such as a platform for minimally invasive procedures, to which instrument 5004 is coupled can comprise any number of engagement positions that correspond to any number of fluid volumes to, for example, effect a given surgical procedure and/or to effect a desired task.

As discussed above, instrument 5004 can comprise any of the head configurations described or depicted in this disclosure that can operate in the same or similar way. In this way, system 5000 may be used in minimally invasive procedures. For example, instrument 5004 can be coupled to a platform for minimally invasive procedures (similar to that depicted in FIG. 6) in order to be controllable by an operator of the platform for minimally invasive procedures. A head comprising or coupled to a needle can be coupled to shaft 5008 such that the head and the needle are in fluid communication with tubing 5012 extending through shaft 5008 and housing 5016 and in fluid communication with fluid reservoir 5024.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 5004, such as a needle, a head, and a portion of shaft 5008 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 5004 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 5004 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 5024 can exit fluid reservoir 5024 due at least in part to gravity, force applied to fluid reservoir 5024, and/or activation of pump 5028 (e.g., via a device, such as a platform for minimally invasive procedures, or via housing 5016). Fluid from fluid reservoir 5024 exits fluid reservoir 5024 through tubing 5012, enters housing 5016 through wall 5026, moves through housing 5016, through shaft 4008, through a head and a needle to which instrument 5004 is coupled, and, if an operator of the platform for minimally invasive procedures has directed instrument 5004 (and, more specifically, a needle) to pierce tissue of a subject, fluid exits the needle into tissue of a subject or a lumen of an organ of the subject. Additionally, in the embodiment shown in FIG. 17, if suction system 5076 and/or suction device 5096 is activated by an operator (e.g., via a device, such as a platform for minimally invasive procedures and/or housing 5016), instrument 5004 is configured to enable an operator to remove fluid and material from the body of a subject (e.g., body 288, as depicted in FIG. 6) through a head to which instrument 5004 is coupled, through tubing 5080 of shaft 5008, and out of housing 5016.

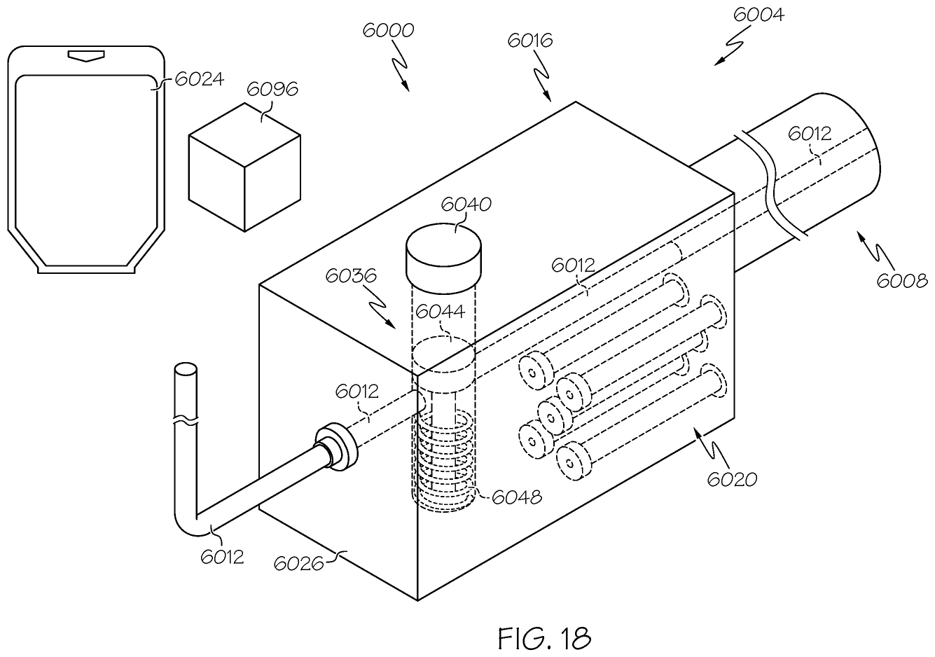

FIG. 18 depicts system 6000 comprising instrument 6004. In the embodiment shown in FIG. 18, system 6000 comprises shaft 6008, a portion of which is depicted in FIG. 18, which is configured to be coupled to a head of the present disclosure. Shaft 6008 is depicted with two wavy solid lines representing a break in the depiction of shaft 6008 to indicate that shaft 6008 can extend in the same manner indefinitely between such wavy solid lines and/or can extend at least as long as necessary between such wavy solid lines in order to effect the described embodiments. Therefore, at least some of shaft 6008 is not drawn to scale.

The embodiment shown in FIG. 18 further comprises tubing 6012, which extends through shaft 6008 and toward a head to which instrument 6004 can be coupled such that shaft 6008 and a head can be in fluid communication. Tubing 6012 includes sets of two wavy solid lines depicting breaks in the depiction of tubing 6012 to indicate that tubing 6012 can extend in the same manner indefinitely between such sets of wavy solid lines and/or can extend at least as long as necessary between such sets of wavy solid lines in order to effect the described embodiments. Therefore, at least some of tubing 6012 is not drawn to scale. Tubing 6012 can comprise the same or similar features and characteristics as those depicted and described with respect to tubing 116.

In the embodiment shown in FIG. 18, shaft 6008 is configured to be coupled to housing 6016 and is depicted extending from and coupled to housing 6016 in the embodiments shown. In the embodiment shown in FIG. 18, a head can further be coupled to shaft 6008 by instrument control system 6020 (or can be indirectly coupled to instrument control system 6020), at least a portion of which is disposed within housing 6016 and at least a portion of which is depicted in FIG. 18. Instrument control system 6020 can comprise the same or similar features and characteristics that operate in the same or similar way as those depicted and described with respect to instrument control system 126, including instrument control shafts, control shaft engagement surfaces, pulleys, and wires, some of which are not depicted in FIG. 18. Instrument control system 6020 can be configured to enable an operator to control instrument 6004, including moving a head with respect to shaft 6008 (e.g., such as an operator of a device, including a platform for minimally invasive procedures).

In the embodiment shown in FIG. 18, instrument 6004 is configured to enable fluid reservoir 6024, which is configured to be coupled to tubing 6012, and a head of the present disclosure to be in fluid communication, such as, for example, via tubing 6012, which can extend from fluid reservoir 6024, through housing 6016, through shaft 6008, and into a head to which instrument 6004 can be coupled. In the embodiment shown, fluid reservoir 6024 is a collapsible bag (e.g., an intravenous fluid bag); however, in other embodiments, fluid reservoir 6024 can comprise a syringe, a vial, and/or any other container configured to accommodate fluid. For example, tubing 6012 extends from a head to which instrument 6004 can be coupled, through a lumen of shaft 6008, through housing 6016, and exits wall 6026 of housing 6016 to enable fluid reservoir 6024, if fluid reservoir 6024 is coupled to tubing 6012, and a head to which instrument 6004 can be coupled to be in fluid communication via housing 6016. In some embodiments, tubing 6012 comprises a fixed connection with housing 6016; and in other embodiments, tubing 6012 is couplable to and/or decouplable from housing 6016. Tubing 6012 can be couplable to and/or decouplable from housing 6016 and fluid reservoir 6024 by any suitable connection, such as, for example, a screw connection, a snap connection, a Luer taper connection, a spike connection in which a piercing device is coupled to a portion of tubing 6012 and can be pierced through a portion of fluid reservoir 6024 or housing 6016, and similar connections. In the embodiment shown, if fluid reservoir 6024 is coupled to tubing 6012, fluid from fluid reservoir 6024 can exit fluid reservoir 6024 and move from fluid reservoir 6024, into and through housing 6016, through a lumen of shaft 6008, and into a head to which instrument 6004 can be coupled. Tubing 6012 can extend from fluid reservoir 6024 to a head to which instrument 6004 can be coupled in one or more pieces of tubing. Fluid within fluid reservoir 6024 can comprise any fluid described above with respect to fluid reservoir 204.

In the embodiment shown, if fluid reservoir 6024 contains fluid and if fluid reservoir 6024 is coupled to tubing 6012, fluid exits fluid reservoir 6024 and can move into housing 6016 via wall 6026, through tubing 6012 extending through housing 6016 and shaft 6008, and into a head to which instrument 6004 can be coupled due at least in part to force of gravity. In the embodiment shown, if fluid reservoir 6024 contains fluid and if fluid reservoir 6024 is coupled to tubing 6012, fluid exits fluid reservoir 6024 and can move into housing 6016 via wall 6026, through tubing 6012 extending through housing 6016 and shaft 6008, and into a head to which instrument 6004 can be coupled due at least in part to force applied to fluid reservoir 6024 by an operator (e.g., by applying manual pressure to fluid reservoir 6024, such as by squeezing fluid reservoir 6024, to assist fluid in exiting fluid reservoir 6024).

In other embodiments, system 6000 can comprise a pump to pump and/or encourage fluid from fluid reservoir 6024 toward a head to which instrument 6004 can be coupled. Such a pump can be disposed exterior to housing 6016 or interior to housing 6016. Such a pump can be couplable to and decouplable from components of system 6000 or in fixed connection with components of system 6000. In such an embodiment, a pump can be a positive displacement pump, a mechanical pump, an electrical pump, a pneumatic pump, an impulse pump, a gravity pump, and any combination thereof. The pump can further be coupled to a power source (e.g., batteries or an electrical source) that is configured to provide power to the pump when activated.

In the embodiment shown in FIG. 18, instrument 6004 and, more specifically, housing 4016, further comprises fluid control system 6036 that is configured to enable housing 6016 to be controllable by an operator to enable fluid to enter shaft 6008 and/or to be controllable by an operator to prevent fluid from entering shaft 6008. For example, in the embodiment shown, fluid control system 6036 comprises button 6040, stop 6044, and spring 6048 coupled to stop 6044. If button 6040 is engaged by an operator, spring 6048 is depressed such that stop 6044 moves. In some embodiments, stop 6044 can be positioned such that, if button 6040 is unengaged, stop 6044 does not interfere with and/or block tubing 6012 such that fluid is not prevented by stop 6044 from entering shaft 6008. In such an embodiment, if an operator engages button 6040 to move stop 6044 to interfere with and/or block tubing 6012, fluid is prevented from entering shaft 6008. In other embodiments, stop 6044 can be positioned such that, if button 6040 is unengaged, stop 6044 interferes with and/or blocks tubing 6012 such that fluid is prevented by stop 6044 from entering shaft 6008. In such an embodiment, if an operator engages button 6040 to move stop 6044 so that it does not interfere with and/or block tubing 6012, fluid is permitted to move into shaft 6008 (e.g., due to a pressure applied on fluid within tubing 6012 via gravity and/or an applied pressure on fluid reservoir 6024). In some embodiments, button 6040 can be controllable by or engageable through a platform for minimally invasive procedures. In some embodiments, button 6040 can comprise various engagement positions in which a first engagement position permits a first volume of fluid to enter shaft 6008, a second engagement position permits a second volume of fluid to enter shaft 6008, and so forth.

Fluid control system 6036 is configured to enable system 6000 to be controllable by an operator to enable fluid to move into the body of a subject through a head to which instrument 6004 can be is coupled. For example, if instrument 6004 is coupled to a device, such as a platform for minimally invasive procedures, that is configured to enable an operator to control instrument 6004, fluid control system 6036 can be coupled to and controllable by such a device to enable an operator of the device to move fluid into the body of a subject through a head to which instrument 6004 can be coupled, such as, for example, by activating fluid control system 6036 (and deactivating fluid control system 6036 to prevent fluid from moving into a body of a subject by fluid control system 6036). Fluid control system 6036 can be configured in various other ways in order to enable movement of fluid into the body of a subject by an operator (such as various combinations of fluid sources, pumps, and stop configurations).

In the embodiment shown in FIG. 18, fluid control system 6036 further operates as a suction system, which is configured to enable fluid and material (e.g., tissue) to be removed from the body of a subject through a head to which instrument 6004 can be coupled. Fluid control system 6036 can be couplable to and decouplable from suction device 6096 (and is depicted decoupled from suction device 6096, in the embodiment shown) via tubing 6012, which exits housing 6016. An operator can alternate between coupling fluid reservoir 6024 to tubing 6012 and suction device 6096 to tubing 6012 depending on, for example, a given surgical procedure and/or to effect a desired task. Tubing 6012 can be couplable to and/or decouplable from suction device 6096 by any suitable connection, such as, for example, a screw connection, a snap connection, a Luer taper connection, a spike connection in which a piercing device is coupled to a portion of tubing 4080 and can be pierced through a portion of suction device 6096, and similar connections. Suction device 6096 can be any device configured to apply pressure to tubing 6012 to enable fluid and material near a head to which instrument 6004 can be coupled to enter a head to which instrument 6004 can be coupled and move through tubing 6012 toward suction device 6096 (e.g., such as a standalone suction device coupled to a power source, such as batteries or an electrical source, or a suction device structurally integrated with a portion of a room (such as through a wall) in which a medical procedure is being performed).

Fluid control system 6036 and suction device 6096 are configured to enable system 6000 to be controllable by an operator to enable removal of fluid and material from the body of a subject through a head to which instrument 6004 is coupled. For example, if instrument 6004 is coupled to a device, such as a platform for minimally invasive procedures, that is configured to enable an operator to control instrument 6004, fluid control system 6036 and/or suction device 6096 can be coupled to and controllable by such a device to enable an operator of the device to remove fluid and material from the body of a subject through a head to which instrument 6004 is coupled, such as, for example, by activating fluid control system 6036 and/or suction device 6096 (and deactivating fluid control system 6036 and/or suction device 6096 to prevent removal of fluid and material through fluid control system 6036 and/or suction device 6096). As another example, in the embodiment shown, if button 6040 is engaged by an operator, spring 6048 is depressed such that stop 6044 moves. In some embodiments, stop 6044 can be positioned such that, if button 6040 is unengaged, stop 6044 does not interfere with and/or block tubing 6012 such that fluid is not prevented by stop 6044 from exiting housing 6016. In such an embodiment, if an operator engages button 6040 to move stop 6044 to interfere with and/or block tubing 6012, fluid and material is prevented from exiting housing 6016 (in some embodiments, fluid and material is prevented from exiting housing 6016 even if suction device 6096 is activated). In other embodiments, stop 6044 can be positioned such that, if button 6040 is unengaged, stop 6044 interferes with and/or blocks tubing 6012 such that fluid is prevented by stop 6044 from exiting housing 6016 (in some embodiments, fluid and material is prevented from exiting housing 6016 even if suction device 6096 is activated). In such an embodiment, if an operator engages button 6040 to move stop 6044 so that it does not interfere with and/or block tubing 6012, fluid is permitted to exit housing 6016, such as, for example, if suction device 6096 is activated. Fluid control system 6036 and/or suction device 6096 can be configured in various other ways in order to enable removal of fluid and material from the body of a subject by an operator (such as various combinations of pressure sources, pumps, and stop configurations).

System 6000 and/or a device to which instrument 6004 is coupled, such as a platform for minimally invasive procedures, can be configured to permit delivery of a predetermined amount of fluid (and/or a substantially equal volume of fluid in succession). For example, if an operator engages button 6040, a predetermined amount of fluid can be permitted to move through tubing 6012 past fluid control system 6036 and into shaft 6008 (and, after such amount of fluid has entered shaft 6008, fluid control system 6036 can be configured to prevent additional fluid from moving past fluid control system 6036). In some embodiments, fluid control system 6036 and/or a device, such as a platform for minimally invasive procedures, to which instrument 6004 is coupled can comprise various engagement positions in which a first engagement position permits a first volume of fluid to enter shaft 6008, a second engagement position permits a second volume of fluid to enter shaft 6008, and so forth. As an example, in such an embodiment, a first engagement position may permit a first volume of fluid to enter shaft 6008 to deliver fluid through tissue of a subject, and a second engagement position may permit a second volume of fluid to enter shaft 6008 to irrigate a target area within the body of a subject. Fluid control system 6036 and/or a device, such as a platform for minimally invasive procedures, to which instrument 6004 is coupled can comprise any number of engagement positions that correspond to any number of fluid volumes to, for example, effect a given surgical procedure and/or to effect a desired task.

As discussed above, instrument 6004 can comprise any of the head configurations described or depicted in this disclosure that can operate in the same or similar way. In this way, system 6000 may be used in minimally invasive procedures. For example, instrument 6004 can be coupled to a platform for minimally invasive procedures (similar to that depicted in FIG. 6) in order to be controllable by an operator of the platform for minimally invasive procedures. A head comprising or coupled to a needle can be coupled to shaft 6008 such that the head and the needle are in fluid communication with tubing 6012 extending through shaft 6008 and housing 6016 and in fluid communication with fluid reservoir 6024.

One or more incision (e.g., three incision 284, as depicted in FIG. 6) can be made in a body of a subject (e.g., body 288, as depicted in FIG. 6), such as in the abdomen, and a trocar (e.g., trocars 292, as depicted in FIG. 6) can be disposed in each incision (e.g., each of incisions 284, as depicted in FIG. 6) to provide access to the interior of the body of the subject (e.g., interior to body 288, as depicted in FIG. 6). At least a portion of instrument 6004, such as a needle, a head, and a portion of shaft 6008 is configured to be disposed through a trocar (e.g., one of trocars 292, as depicted in FIG. 6) into a body of a subject (e.g., body 288, as depicted in FIG. 6), and a platform for minimally invasive procedures (e.g., such as arm 280, as depicted in FIG. 6) can be docked to a trocar (e.g., one or more of trocars 292, as depicted in FIG. 6). If disposed through a trocar (e.g., one of trocars 292, as depicted in FIG. 6) into a body of a subject (e.g., body 288, as depicted in FIG. 6), instrument 6004 is configured to permit delivery of fluid through tissue of a subject. For example, in some embodiments, tissue of a subject can be pierced by a needle to permit delivery of fluid through the tissue. In such an embodiment, “through the tissue” can mean through a surface layer of tissue of a subject and into at least a portion of an abdominal wall of a subject (e.g., into fascia or within the fascial planes). In such an embodiment, “through the tissue” can further mean through a surface layer of tissue of an organ (e.g., a gallbladder or other hollow body) of a subject and into a lumen of the organ. If disposed through a trocar (e.g., one of trocars 292, as depicted in FIG. 6) into a body of a subject (e.g., body 288, as depicted in FIG. 6), instrument 6004 is further configured to permit removal of fluid through tissue of a subject. For example, in some embodiments, tissue of a subject can be pierced by a needle to permit removal of fluid through the tissue. In such an embodiment, “through the tissue” can mean from at least a portion of an abdominal wall of a subject (e.g., from fascia) and through a surface layer of tissue of a subject. In such an embodiment, “through the tissue” can further mean from a lumen of an organ (e.g., a gallbladder or other hollow body) of a subject and through a surface layer of tissue of the organ. As described above, fluid from fluid reservoir 6024 can exit fluid reservoir 6024 due at least in part to gravity, force applied to fluid reservoir 6024, and/or activation of a pump to which system 6000 can be coupled (e.g., via a device, such as a platform for minimally invasive procedures, or via housing 6016). Fluid from fluid reservoir 6024 exits fluid reservoir 6024 through tubing 6012, enters housing 6016 through wall 6026, moves through fluid control system 6036, through shaft 6008, through a head and a needle to which instrument 6004 is coupled, and, if an operator of the platform for minimally invasive procedures has directed instrument 6004 (and, more specifically, a needle) to pierce tissue of a subject, fluid exits the needle into tissue of a subject or a lumen of an organ of the subject. Additionally, in the embodiment shown in FIG. 18, if suction device 6096 is coupled to tubing 6012 and is activated by an operator (e.g., via a device, such as a platform for minimally invasive procedures and/or housing 6016), instrument 6004 is configured to enable an operator to remove fluid and material from the body of a subject (e.g., body 288, as depicted in FIG. 6) through a head to which instrument 6004 is coupled (and, in some embodiments, through a needle that a head comprises or to which a head is coupled), through tubing 6080 of shaft 6008, and out of housing 6016.

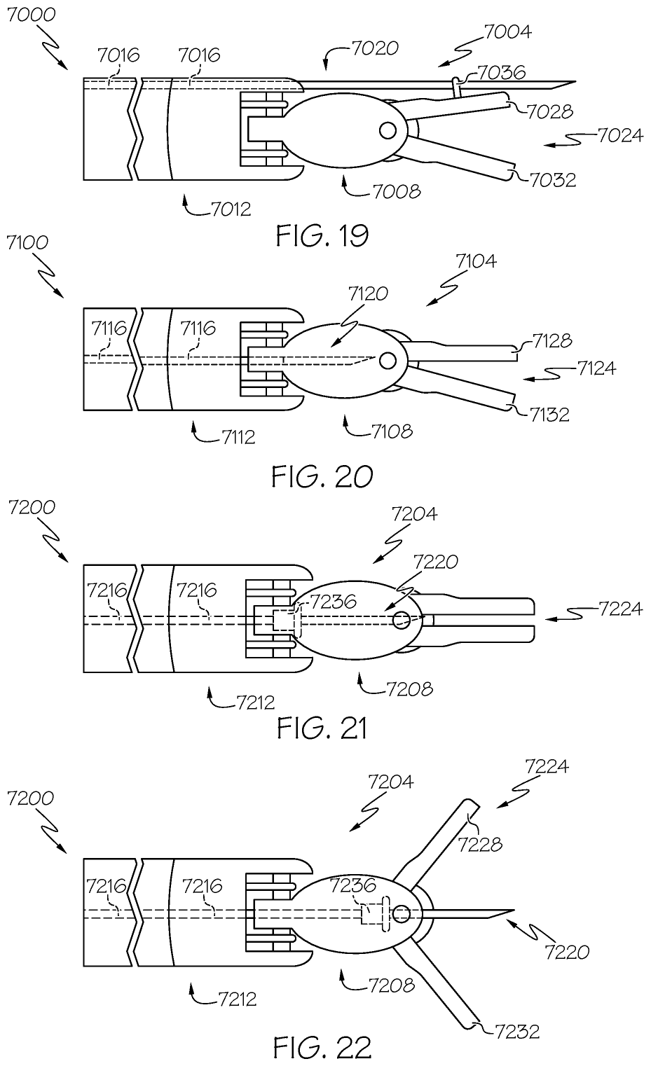

FIGS. 19-24 depict additional examples of the present instruments, systems, methods, and components thereof. As explained above, aspects of any of the embodiments depicted or described above or below may be combined with aspects of the embodiments depicted and described with respect to FIGS. 19-24 to form further embodiments having comparable or different properties and addressing the same or different problems. The embodiments depicted in FIGS. 19-24 and described below can be modified, adapted, and/or supplemented to comprise any of the features shown or described with respect to any of the other systems, instruments, or methods in this disclosure. Similarly, embodiments depicted in other figures or described with respect thereto can be modified, adapted, and/or supplemented to comprise any of the features shown or described with respect to the systems, instruments, and methods of FIGS. 19-24. One or more features of FIGS. 19-24 having the same or similar reference numeral as other features of other embodiments in this disclosure should not be construed to indicate that any feature is limited to the characteristics of another feature having the same or similar reference numeral, or that any feature cannot already have, or cannot be modified to have, features that are different from another feature having the same or similar reference numeral. Though the same or similar feature may have a detailed description with respect to a first embodiment, a second embodiment having the same or similar feature without such a detailed description of such feature (or without any description of such feature) should not be understood to preclude such feature of the second embodiment from comprising the same or similar characteristics as depicted or described with respect to the feature of the first embodiment. For the sake of brevity, all features that an embodiment has or could have may not be described in each separate embodiment.

As discussed above, systems and instruments of this disclosure can comprise and/or be coupled to various heads. FIGS. 19-24 depicts at least a portion of the instruments described in this disclosure and, in particular, various examples of such heads, including components, systems, and methods relating thereto. Any of the systems and methods of this disclosure can be modified, adapted, and/or supplemented to comprise any such head of FIGS. 19-24, including components, systems, and methods relating thereto, in addition to any other head configurations, component, system, or methods relating thereto described or depicted throughout this disclosure and can operate in the same or similar way as described. In this way, the heads, including components, systems, and methods relating thereto of FIGS. 19-24 may be used, for example, in minimally invasive procedures as described in this disclosure. Similarly, any of the below heads can comprise the same or similar features or be coupled to the same or similar components as those heads described with respect to other figures. For example, the heads described below can be movable and rotatable in the same way as the head described above.

FIG. 19 depicts system 7000 comprising a portion of instrument 7004. In the embodiment shown in FIG. 19, system 7000 comprises head 7008, which is configured to be coupled to shaft 7012 and is depicted coupled to shaft 7012 in the embodiment shown. In FIG. 19, shaft 7012 is depicted with two wavy solid lines representing a break in the depiction of shaft 7012 to indicate that shaft 7012 can extend in the same manner indefinitely between such wavy solid lines and/or can extend at least as long as necessary between such wavy solid lines in order to effect the described embodiments. Therefore, at least some of shaft 7012 is not drawn to scale. Head 7008 can be coupled to shaft 7012 in any suitable way, including in any suitable way to enable movement of head 7008 with respect to shaft 7012, such as, for example, by one or more pins, screws, hinges, adhesive, bolts, and the like. In some embodiments, head 7008 is decouplable from shaft 7012 to enable replacement of head 7008 with a head having similar functionality and/or replacement of head 7008 with a head having different functionality. In other embodiments, head 7008 is unitary with shaft 7012 (e.g., at least partially formed of a single piece of material and, therefore, not couplable to or decouplable from shaft 7012).

In the embodiment shown in FIG. 19, instrument 7004 further comprises tubing 7016 extending through a lumen of shaft 7012. In the embodiment shown, tubing 7016 is configured to be coupled to needle 7020 (and is depicted coupled to needle 7020, in the embodiment shown) to enable shaft 7012 (and, more specifically, tubing 7016 extending through a lumen of shaft 7012) and needle 7020 to be in fluid communication (and are depicted in fluid communication, in the embodiment shown) via tubing 7016. In some embodiments, needle 7020 is in fixed connection with tubing 7016 (such that needle 7020 cannot be decoupled from tubing 7016); and, in other embodiments, needle 7020 is couplable to and decouplable from tubing 7016. Tubing 7016 includes two wavy solid lines depicting breaks in the depiction of tubing 7016 to indicate that tubing 7016 can extend in the same manner indefinitely between such wavy solid lines and/or can extend at least as long as necessary between such wavy solid lines in order to effect the described embodiments. Therefore, at least some of tubing 7016 is not drawn to scale. Tubing 7016 can comprise the same or similar features and characteristics as those depicted and described with respect to tubing 116.

In the embodiment shown in FIG. 19, head 7008 comprises suture grasper 7024 having first jaw 7028 and second jaw 7032, where at least one of first jaw 7028 and second jaw 7032 are movable to enable grasping and releasing of a device for suturing tissue of a subject—a “suturing device” (e.g., such as a needle coupled to suture). Though suture grasper 7024 may primarily be used for grasping and releasing suturing devices, suture grasper 7024 can grasp, release, and/or otherwise move other items, including tissue, instruments, prosthetics, and the like. As discussed above, head 7008 can be coupled to an instrument control system (similar to instrument control system 126) to enable an operator to move head 7008, including moving at least one of first jaw 7028 and second jaw 7032, moving head 7008 with respect to shaft 7012 (as described in detail with respect to other embodiments), and rotating head 7008 with respect to shaft 7012 (as described in detail with respect to other embodiments). For example, in some embodiments, instrument 7004 can be configured to enable movement of first jaw 7028 toward second jaw 7032 such that, if a suturing device is positioned between first jaw 7028 and second jaw 7032, first jaw 7028 can secure the suturing device between first jaw 7028 and second jaw 7032 by applying a force on the suturing device in the direction of second jaw 7032 to enable an operator of instrument 7004 to suture a patient with the suturing device. Similarly, in some embodiments, instrument 7004 can be configured to enable movement of second jaw 7032 toward first jaw 7028 such that, if a suturing device is positioned between first jaw 7028 and second jaw 7032, second jaw 7032 can secure the suturing device between first jaw 7028 and second jaw 7032 by applying a force on the suturing device in the direction of first jaw 7028 to enable an operator of instrument 7004 to suture a patient with the suturing device. As another example, in some embodiments, instrument 7004 can be configured to enable movement of second jaw 7032 toward first jaw 7028 and first jaw 7028 toward second jaw 7032 such that, if a suturing device is positioned between first jaw 7028 and second jaw 7032, second jaw 7032 and first jaw 7028 can secure the suturing device between first jaw 7028 and second jaw 7032 by each applying a force on the suturing device in the direction of the other jaw to enable an operator of instrument 7004 to suture a patient with the suturing device.

In the embodiment shown in FIG. 19, head 7008 comprises eyehole 7036 extending from head 7008 and having an opening configured to enable needle 7020 to advance through the opening of eyehole 7036. In the embodiment shown, eyehole 7036 extends from first jaw 7028 of suture grasper 7024; however, in other embodiments, eyehole 7036 can extend from second jaw 7032 of suture grasper 7024; and, in still other embodiments, eyehole 7036 can extend from another portion of head 7008 or from a portion of shaft 7012. If at least a portion of needle 7020 extends through eyehole 7036, as depicted in FIG. 19, needle 7020 is disposed in fixed relation to head 7008 such that needle 7020 moves and/or rotates with respect to shaft 7012 if head 7008 moves and/or rotates with respect to shaft 7012 and in substantially the same manner and direction in which head 7008 moves and/or rotates with respect to shaft 7012.

In some embodiments, at least a portion of needle 7020 extends through eyehole 7036 and is fixed such that needle 7020 cannot advance from shaft 7012 and/or retract into shaft 7012. In other embodiments, such as the embodiment shown, instrument 7004 is configured to enable at least a portion of needle 7020 to retract into shaft 7012 and further configured to enable at least a portion of needle 7020 to advance from shaft 7012 (e.g., at a direction of an operator). For example, in a retracted configuration, at least a portion of needle 7020 does not extend through eyehole 7036. Instrument 7004 is configured to enable at least a portion of needle 7020 to be advanced from shaft 7012 (e.g., via an opening in a portion of shaft 7012) such that needle 7020 advances through eyehole 7036 to dispose needle 7020 in fixed relation to head 1008. As depicted in the embodiment shown, such an advancement enables needle 7020 to extend beyond first jaw 7028 and second jaw 7032 of suture grasper 7024, which can permit needle 7020 to pierce tissue of a subject without interference from suture grasper 7024 and/or another component of instrument 7004. From such an advanced configuration, instrument 7004 is configured to enable at least a portion of needle 7020 to be retracted into shaft 7012 such that needle 7020 does not extend through eyehole 7036. Such a retracted configuration enables first jaw 7028 and second jaw 7032 of suture grasper 7024 to extend beyond needle 7020, which can permit suturing of a subject with suture grasper 7024 without interference from needle 7020. Instrument 7004 is configured to enable advancement and retraction of needle 7020 by an operator (e.g., via an instrument control system, such as that described with respect to instrument control system 126), including an operator of a device, such as a platform for minimally invasive procedures. Various manners in which a needle can be advanced and/or retracted are described in detail below. Tubing 7016 can be configured to advance and retract with needle 7020 to enable needle 7020 and tubing 7016 to remain in fluid communication.

As discussed above, shaft 7012 of instrument 7004 can be coupled to and in fluid communication with a housing such that the housing is in fluid communication with needle 7020. Further, instrument 7004 is configured to be coupled to a fluid reservoir (such as, for example, via tubing 7016 and/or through a housing to which instrument 7004 is coupled) to enable the fluid reservoir to be in fluid communication with shaft 7012 and, therefore, in fluid communication with needle 7020. As with instruments described above, at least a portion of instrument 7004 is configured to be disposed through a trocar (e.g., one of trocars 292, as depicted in FIG. 6) into a body of a subject (e.g., body 288, as depicted in FIG. 6). At least a portion of instrument 7004 is further configured to be coupled to a platform for minimally invasive procedures and controllable by an operator of the platform for minimally invasive procedures (similar to that depicted in FIG. 6), and the platform for minimally invasive procedures (e.g., such as arm 280, as depicted in FIG. 6) can be docked to a trocar (e.g., one or more of trocars 292, as depicted in FIG. 6). If at least a portion of instrument 7004 is disposed through a trocar into a body of a subject, if a fluid reservoir is in fluid communication with shaft 7012, and if shaft 7012 is in fluid communication with needle 7020, instrument 7004 is configured to permit delivery of fluid (e.g., at the direction of an operator) from the fluid reservoir, through shaft 7012, through needle 7020, and through tissue of a subject (as described in detail above). Furthermore, instrument 7004 is configured to permit suturing of tissue of a subject with suture grasper 7024 (e.g., at the direction of an operator).

FIG. 20 depicts system 7100 comprising a portion of instrument 7104. In the embodiment shown in FIG. 20, system 7100 comprises head 7108, which is configured to be coupled to shaft 7112 and is depicted coupled to shaft 7112 in the embodiment shown. In FIG. 20, shaft 7112 is depicted with two wavy solid lines representing a break in the depiction of shaft 7112 to indicate that shaft 7112 can extend in the same manner indefinitely between such wavy solid lines and/or can extend at least as long as necessary between such wavy solid lines in order to effect the described embodiments. Therefore, at least some of shaft 7112 is not drawn to scale. Head 7108 can be coupled to shaft 7112 in any suitable way, including in any suitable way to enable movement of head 7108 with respect to shaft 7112, such as, for example, by one or more pins, screws, hinges, adhesive, bolts, and the like. In some embodiments, head 7108 is decouplable from shaft 7112 to enable replacement of head 7108 with a head having similar functionality and/or replacement of head 7108 with a head having different functionality. In other embodiments, head 7108 is unitary with shaft 7112 (e.g., at least partially formed of a single piece of material and, therefore, not couplable to or decouplable from shaft 7112).

In the embodiment shown in FIG. 20, instrument 7114 further comprises tubing 7116 extending through a lumen of shaft 7112. In the embodiment shown, tubing 7116 is configured to be coupled to needle 7120 (and is depicted coupled to needle 7120, in the embodiment shown) to enable shaft 7112 (and, more specifically, tubing 7116 extending through a lumen of shaft 7112) and needle 7120 to be in fluid communication (and are depicted in fluid communication, in the embodiment shown) via tubing 7116. Further, tubing 7116 extends from shaft 7112 into head 7108 such that shaft 7112, head 7108, and needle 7120 are in fluid communication, if needle 7120 is coupled to tubing 7116. In some embodiments, needle 7120 is in fixed connection with tubing 7116 (such that needle 7120 cannot be decoupled from tubing 7116); and, in other embodiments, needle 7120 is couplable to and decouplable from tubing 7116. Tubing 7116 includes two wavy solid lines depicting breaks in the depiction of tubing 7116 to indicate that tubing 7116 can extend in the same manner indefinitely between such wavy solid lines and/or can extend at least as long as necessary between such wavy solid lines in order to effect the described embodiments. Therefore, at least some of tubing 7116 is not drawn to scale. Tubing 7116 can comprise the same or similar features and characteristics as those depicted and described with respect to tubing 116.

In the embodiment shown in FIG. 20, head 7108 comprises suture grasper 7124 having first jaw 7128 and second jaw 7132, where at least one of first jaw 7128 and second jaw 7132 are movable to enable grasping and releasing of a device for suturing tissue of a subject—a “suturing device” (e.g., such as a needle coupled to suture). Though suture grasper 7124 may primarily be used for grasping and releasing suturing devices, suture grasper 7124 can grasp, release, and/or otherwise move other items, including tissue, instruments, prosthetics, and the like. As discussed above, head 7108 can be coupled to an instrument control system (similar to instrument control system 126) to enable an operator to move head 7108, including moving at least one of first jaw 7128 and second jaw 7132, moving head 7108 with respect to shaft 7112 (as described in detail with respect to other embodiments), and rotating head 7108 with respect to shaft 7112 (as described in detail with respect to other embodiments). For example, in some embodiments, instrument 7104 can be configured to enable movement of first jaw 7128 toward second jaw 7132 such that, if a suturing device is positioned between first jaw 7128 and second jaw 7132, first jaw 7128 can secure the suturing device between first jaw 7128 and second jaw 7132 by applying a force on the suturing device in the direction of second jaw 7132 to enable an operator of instrument 7104 to suture a patient with the suturing device. Similarly, in some embodiments, instrument 7104 can be configured to enable movement of second jaw 7132 toward first jaw 7128 such that, if a suturing device is positioned between first jaw 7128 and second jaw 7132, second jaw 7132 can secure the suturing device between first jaw 7128 and second jaw 7132 by applying a force on the suturing device in the direction of first jaw 7128 to enable an operator of instrument 7104 to suture a patient with the suturing device. As another example, in some embodiments, instrument 7104 can be configured to enable movement of second jaw 7132 toward first jaw 7128 and first jaw 7128 toward second jaw 7132 such that, if a suturing device is positioned between first jaw 7128 and second jaw 7132, second jaw 7132 and first jaw 7128 can secure the suturing device between first jaw 7128 and second jaw 7132 by each applying a force on the suturing device in the direction of the other jaw to enable an operator of instrument 7104 to suture a patient with the suturing device.

In the embodiment shown in FIG. 20, instrument 7104 is configured to enable needle 7120 to be advanced between first jaw 7128 and second jaw 7132 of suture grasper 7124 in order to dispose needle 7120 in fixed relation to head 7108 such that needle 7120 moves and/or rotates with respect to shaft 7112 if head 7108 moves and/or rotates with respect to shaft 7112 and in substantially the same manner and direction in which head 7108 moves and/or rotates with respect to shaft 7112. For example, in the embodiment shown, head 7108 comprises an opening through which at least some of needle 7120 (and, in some embodiments, at least some of tubing 7116) can advance and/or retract (e.g., at a direction of an operator). For example, in a retracted configuration, which is depicted in the embodiment shown in FIG. 20, at least a portion of needle 7120 does not extend through an opening in head 7108 or, alternatively, a portion of needle 7120 extends through an opening in head 7108, while first jaw 7128 and second jaw 7132 of suture grasper 7124 extend beyond needle 7120. Instrument 7104 is configured to enable at least a portion of needle 7120 to be advanced such that needle 7120 extends between first jaw 7128 and second jaw 7132 of suture grasper 7124 or otherwise extends from head 7108 in order to dispose needle 7120 in fixed relation to head 7108. Such an advancement enables needle 7120 to extend beyond first jaw 7128 and second jaw 7132 of suture grasper 7124, which can permit needle 7120 to pierce tissue of a subject without interference from suture grasper 7124 and/or another component of instrument 7104. From such an advanced configuration, instrument 7104 is configured to enable at least a portion of needle 7120 and/or tubing 7116 to be retracted into shaft 7112. Such a retracted configuration enables first jaw 7128 and second jaw 7132 of suture grasper 7124 to extend beyond needle 7120, which can permit suturing of a subject with suture grasper 7124 without interference from needle 7120. Instrument 7104 is configured to enable advancement and retraction of needle 7120 by an operator (e.g., via an instrument control system, such as that described with respect to instrument control system 126), including an operator of a device, such as a platform for minimally invasive procedures. Various manners in which a needle can be advanced and/or retracted are described in detail below. Tubing 7116 can be configured to advance and retract with needle 7120 to enable needle 7120 and tubing 7116 to remain in fluid communication.

As discussed above, shaft 7112 of instrument 7104 can be coupled to and in fluid communication with a housing such that the housing is in fluid communication with needle 7120. Further, instrument 7104 is configured to be coupled to a fluid reservoir (such as, for example, via tubing 7116 and/or through a housing to which instrument 7104 is coupled) to enable the fluid reservoir to be in fluid communication with shaft 7112 and, therefore, in fluid communication with needle 7120. As with instruments described above, at least a portion of instrument 7104 is configured to be disposed through a trocar (e.g., one of trocars 292, as depicted in FIG. 6) into a body of a subject (e.g., body 288, as depicted in FIG. 6). At least a portion of instrument 7104 is further configured to be coupled to a platform for minimally invasive procedures and controllable by an operator of the platform for minimally invasive procedures (similar to that depicted in FIG. 6), and the platform for minimally invasive procedures (e.g., such as arm 280, as depicted in FIG. 6) can be docked to a trocar (e.g., one or more of trocars 292, as depicted in FIG. 6). If at least a portion of instrument 7104 is disposed through a trocar into a body of a subject, if a fluid reservoir is in fluid communication with shaft 7112, and if shaft 7112 is in fluid communication with needle 7120, instrument 7104 is configured to permit delivery of fluid (e.g., at the direction of an operator) from the fluid reservoir, through shaft 7112, through head 7108, through needle 7120, and through tissue of a subject (as described in detail above). Furthermore, instrument 7104 is configured to permit suturing of tissue of a subject with suture grasper 7124 (e.g., at the direction of an operator).

FIGS. 21-22 depicts system 7200 comprising a portion of instrument 7204. In the embodiment shown in FIGS. 21-22, system 7200 comprises head 7208, which is configured to be coupled to shaft 7212 and is depicted coupled to shaft 7212 in the embodiment shown. In FIGS. 21-22, shaft 7212 is depicted with two wavy solid lines representing a break in the depiction of shaft 7212 to indicate that shaft 7212 can extend in the same manner indefinitely between such wavy solid lines and/or can extend at least as long as necessary between such wavy solid lines in order to effect the described embodiments. Therefore, at least some of shaft 7212 is not drawn to scale. Head 7208 can be coupled to shaft 7212 in any suitable way, including in any suitable way to enable movement of head 7208 with respect to shaft 7212, such as, for example, by one or more pins, screws, hinges, adhesive, bolts, and the like. In some embodiments, head 7208 is decouplable from shaft 7212 to enable replacement of head 7208 with a head having similar functionality and/or replacement of head 7208 with a head having different functionality. In other embodiments, head 7208 is unitary with shaft 7212 (e.g., at least partially formed of a single piece of material and, therefore, not couplable to or decouplable from shaft 7212).

In the embodiment shown in FIGS. 21-22, instrument 7204 further comprises tubing 7216 extending through a lumen of shaft 7212. In the embodiment shown, tubing 7216 is configured to be coupled to needle 7220 (and is depicted coupled to needle 7220, in the embodiment shown) to enable shaft 7212 (and, more specifically, tubing 7216 extending through a lumen of shaft 7212) and needle 7220 to be in fluid communication (and are depicted in fluid communication, in the embodiment shown) via tubing 7216. Further, tubing 7216 extends from shaft 7212 into head 7208 such that shaft 7212, head 7208, and needle 7220 are in fluid communication, if needle 7220 is coupled to tubing 7216. In some embodiments, needle 7220 is in fixed connection with tubing 7216 (such that needle 7220 cannot be decoupled from tubing 7216); and, in other embodiments, needle 7220 is couplable to and decouplable from tubing 7216. Tubing 7216 includes two wavy solid lines depicting breaks in the depiction of tubing 7216 to indicate that tubing 7216 can extend in the same manner indefinitely between such wavy solid lines and/or can extend at least as long as necessary between such wavy solid lines in order to effect the described embodiments. Therefore, at least some of tubing 7216 is not drawn to scale. Tubing 7216 can comprise the same or similar features and characteristics as those depicted and described with respect to tubing 116.

In the embodiment shown in FIGS. 21-22, head 7208 comprises suture grasper 7224 having first jaw 7228 and second jaw 7232, where at least one of first jaw 7228 and second jaw 7232 are movable to enable grasping and releasing of a device for suturing tissue of a subject—a “suturing device” (e.g., such as a needle coupled to suture). Though suture grasper 7224 may primarily be used for grasping and releasing suturing devices, suture grasper 7224 can grasp, release, and/or otherwise move other items, including tissue, instruments, prosthetics, and the like. As discussed above, head 7208 can be coupled to an instrument control system (similar to instrument control system 126) to enable an operator to move head 7208, including moving at least one of first jaw 7228 and second jaw 7232, moving head 7208 with respect to shaft 7212 (as described in detail with respect to other embodiments), and rotating head 7208 with respect to shaft 7212 (as described in detail with respect to other embodiments). For example, in some embodiments, instrument 7204 can be configured to enable movement of first jaw 7228 toward second jaw 7232 such that, if a suturing device is positioned between first jaw 7228 and second jaw 7232, first jaw 7228 can secure the suturing device between first jaw 7228 and second jaw 7232 by applying a force on the suturing device in the direction of second jaw 7232 to enable an operator of instrument 7204 to suture a patient with the suturing device. Similarly, in some embodiments, instrument 7204 can be configured to enable movement of second jaw 7232 toward first jaw 7228 such that, if a suturing device is positioned between first jaw 7228 and second jaw 7232, second jaw 7232 can secure the suturing device between first jaw 7228 and second jaw 7232 by applying a force on the suturing device in the direction of first jaw 7228 to enable an operator of instrument 7204 to suture a patient with the suturing device. As another example, in some embodiments, instrument 7204 can be configured to enable movement of second jaw 7232 toward first jaw 7228 and first jaw 7228 toward second jaw 7232 such that, if a suturing device is positioned between first jaw 7228 and second jaw 7232, second jaw 7232 and first jaw 7228 can secure the suturing device between first jaw 7228 and second jaw 7232 by each applying a force on the suturing device in the direction of the other jaw to enable an operator of instrument 7204 to suture a patient with the suturing device.

In the embodiment shown in FIGS. 21-22, needle 7020 is disposed within head 7208 and, therefore, needle 7220 in fixed relation to head 7208 such that needle 7220 moves and/or rotates with respect to shaft 7212 if head 7208 moves and/or rotates with respect to shaft 7212 and in substantially the same manner and direction in which head 7208 moves and/or rotates with respect to shaft 7212.

In the embodiment shown in FIGS. 21-22, instrument 7204 is configured to enable needle 7220 to extend beyond first jaw 7228 and second jaw 7232 to permit needle 7220 to pierce tissue of a subject without interference from suture grasper 7224 and/or another component of instrument 7204. For example, in some embodiments, needle 7220 is coupled in fixed relation to head 7208 such that needle 7220 does not move (e.g., advance or retract) within head 7208 and/or shaft 7212. In such an embodiment, instrument 7204 is configured to move first jaw 7228 and second jaw 7232 of suture grasper 7224 away from each other (e.g., open) a sufficient distance until needle 7220 extends beyond first jaw 7228 and second jaw 7232 of suture grasper 7224, as depicted in FIG. 22. In such an open configuration, needle 7220 extends beyond first jaw 7228 and second jaw 7232 to permit needle 7220 to pierce tissue of a subject without interference from suture grasper 7224 and/or another component of instrument 7204. Instrument 7204 is further configured to move first jaw 7228 and second jaw 7232 of suture grasper 7224 toward each other (e.g., close) a sufficient distance until first jaw 7228 and second jaw 7232 of suture grasper 7224 extend beyond needle 7220, which can permit suturing of a subject with suture grasper 7224 without interference from needle 7220.

In other embodiments, instrument 7204 is configured to enable needle 7220 to extend beyond first jaw 7228 and second jaw 7232 to permit needle 7220 to pierce tissue of a subject without interference from suture grasper 7224 and/or another component of instrument 7204 by enabling needle 7220 to be advanced and retracted. For example, instrument 7204 is configured to enable at least a portion of needle 7220 to be advanced such that needle 7220 extends between first jaw 7228 and second jaw 7232 of suture grasper 7224 or otherwise extends from head 7208. Such an advancement enables needle 7220 to extend beyond first jaw 7228 and second jaw 7232 of suture grasper 7224, which can permit needle 7220 to pierce tissue of a subject without interference from suture grasper 7224 and/or another component of instrument 7204. In the embodiment shown, needle 7220 can be coupled to stop 7236 to limit the extent to which needle 7220 can extend beyond first jaw 7228 and second jaw 7232 of suture grasper 7224. From such an advanced configuration, instrument 7204 is configured to enable needle 7220 to be retracted. Such a retracted configuration enables first jaw 7228 and second jaw 7232 of suture grasper 7224 to extend beyond needle 7220, which can permit suturing of a subject with suture grasper 7224 without interference from needle 7220. In a retracted configuration, to the extent necessary, stop 7236 can further operate as a seal to prevent any fluid and/or material from entering or exiting shaft 7212. Instrument 7204 is configured to enable advancement and retraction of needle 7220 by an operator (e.g., via an instrument control system, such as that described with respect to instrument control system 126), including an operator of a device, such as a platform for minimally invasive procedures. Various manners in which a needle can be advanced and/or retracted are described in detail below. Tubing 7216 can be configured to advance and retract with needle 7220 to enable needle 7220 and tubing 7216 to remain in fluid communication.

As discussed above, shaft 7212 of instrument 7204 can be coupled to and in fluid communication with a housing such that the housing is in fluid communication with needle 7220. Further, instrument 7204 is configured to be coupled to a fluid reservoir (such as, for example, via tubing 7216 and/or through a housing to which instrument 7204 is coupled) to enable the fluid reservoir to be in fluid communication with shaft 7212 and, therefore, in fluid communication with needle 7220. As with instruments described above, at least a portion of instrument 7204 is configured to be disposed through a trocar (e.g., one of trocars 292, as depicted in FIG. 6) into a body of a subject (e.g., body 288, as depicted in FIG. 6). At least a portion of instrument 7204 is further configured to be coupled to a platform for minimally invasive procedures and controllable by an operator of the platform for minimally invasive procedures (similar to that depicted in FIG. 6), and a platform for minimally invasive procedures (e.g., such as arm 280, as depicted in FIG. 6) can be docked to a trocar (e.g., one or more of trocars 292, as depicted in FIG. 6). If at least a portion of instrument 7204 is disposed through a trocar into a body of a subject, if a fluid reservoir is in fluid communication with shaft 7212, and if shaft 7212 is in fluid communication with needle 7220, instrument 7204 is configured to permit delivery of fluid (e.g., at the direction of an operator) from the fluid reservoir, through shaft 7212, through head 7208, through needle 7220, and through tissue of a subject (as described in detail above). Furthermore, instrument 7204 is configured to permit suturing of tissue of a subject with suture grasper 7224 (e.g., at the direction of an operator).

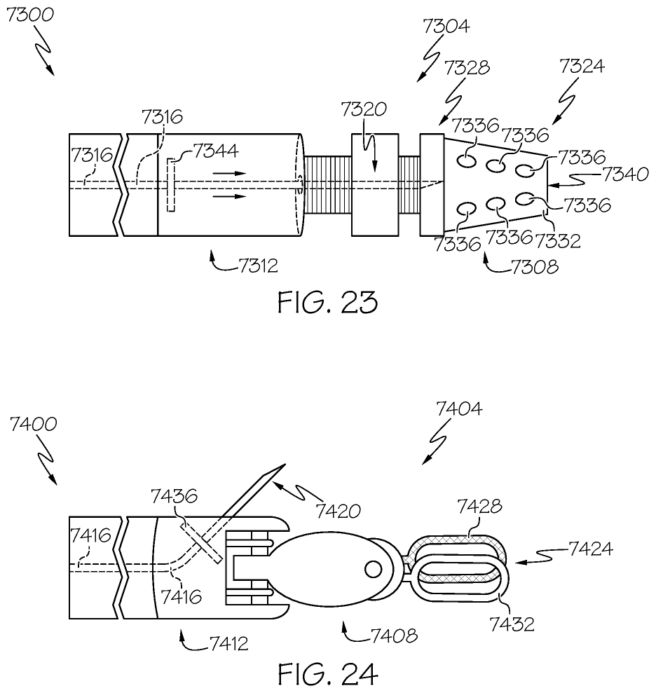

FIG. 23 depicts system 7300 comprising a portion of instrument 7304. In the embodiment shown in FIG. 23, system 7300 comprises head 7308, which is configured to be coupled to shaft 7312 and is depicted coupled to shaft 7312 in the embodiment shown. In FIG. 23, shaft 7312 is depicted with two wavy solid lines representing a break in the depiction of shaft 7312 to indicate that shaft 7312 can extend in the same manner indefinitely between such wavy solid lines and/or can extend at least as long as necessary between such wavy solid lines in order to effect the described embodiments. Therefore, at least some of shaft 7312 is not drawn to scale. Head 7308 can be coupled to shaft 7312 in any suitable way, including in any suitable way to enable movement of head 7308 with respect to shaft 7312, such as, for example, by one or more pins, screws, hinges, adhesive, bolts, and the like, and/or coupled by or formed from the same or similar material as the shaft. In some embodiments, head 7308 is decouplable from shaft 7312 to enable replacement of head 7308 with a head having similar functionality and/or replacement of head 7308 with a head having different functionality. In other embodiments, head 7308 is unitary with shaft 7312 (e.g., at least partially formed of a single piece of material and, therefore, not couplable to or decouplable from shaft 7312).

In the embodiment shown in FIG. 23, instrument 7314 further comprises tubing 7316 extending through a lumen of shaft 7312. In the embodiment shown, tubing 7316 is configured to be coupled to needle 7320 (and is depicted coupled to needle 7320, in the embodiment shown) to enable shaft 7312 (and, more specifically, tubing 7316 extending through a lumen of shaft 7312) and needle 7320 to be in fluid communication (and are depicted in fluid communication, in the embodiment shown) via tubing 7316. Further, tubing 7316 can be configured to extends from shaft 7312 into head 7308 such that shaft 7312, head 7308, and needle 7320 are in fluid communication, if needle 7320 is coupled to tubing 7316. In some embodiments, needle 7320 is in fixed connection with tubing 7316 (such that needle 7320 cannot be decoupled from tubing 7316); and, in other embodiments, needle 7320 is couplable to and decouplable from tubing 7316. Tubing 7316 includes two wavy solid lines depicting breaks in the depiction of tubing 7316 to indicate that tubing 7316 can extend in the same manner indefinitely between such wavy solid lines and/or can extend at least as long as necessary between such wavy solid lines in order to effect the described embodiments. Therefore, at least some of tubing 7316 is not drawn to scale. Tubing 7316 can comprise the same or similar features and characteristics as those depicted and described with respect to tubing 116.

In the embodiment shown in FIG. 23, head 7308 comprises first end 7324, second end 7328, and surface 7332 extending between first end 7324 and second end 7328. Surface 7332 comprises openings 7336, which are configured to enable fluid to enter or exit head 7308. In the embodiment shown, second end 7328 of head 7308 is coupled to shaft 7312. In some embodiments, second end 7328 of head 7308 is couplable to and decouplable from shaft 7312; and in other embodiments, second end 7328 of head 7308 is in fixed connection with shaft 7312 (e.g., such that second end 7328 of head 7308 cannot be decoupled from shaft 7312). First end 7324 of head 7308 comprises end opening 7340, which is configured to enable fluid to enter or exit head 7308. In the embodiment shown, first end 7324 of head 7308 comprises a smaller diameter than second end 7328 of head 7308 and, therefore, surface 7332 extending between first end 7324 and second end 7328 of head 7308 is frustoconically-shaped. In other embodiments, surface 7332 can comprise any suitable shape, such as cylindrical or cubical, to enable fluid to enter or exit head 7308 (e.g., through openings 7336 and/or end opening 7340). Instrument 7304 can operate in the same or similar ways as the instruments described in detail in FIGS. 14-18.

In the embodiment shown in FIG. 23, instrument 7304 is configured to enable needle 7320 to be advanced within shaft 7312 and/or head 7308 in order to dispose needle 7320 in fixed relation to head 7308 such that needle 7320 moves and/or rotates with respect to shaft 7312 if head 7308 moves and/or rotates with respect to shaft 7312 and in substantially the same manner and direction in which head 7308 moves and/or rotates with respect to shaft 7312. For example, in the embodiment shown, shaft 7312 and/or head 7308 comprises an opening through which needle 7320 (and, in some embodiments, at least some of tubing 7316) can advance and/or retract (e.g., at a direction of an operator). For example, in a retracted configuration, which is depicted in the embodiment shown in FIG. 23, at least a portion of needle 7320 does not extend through end opening 7340 in head 7308. Instrument 7304 is configured to enable at least a portion of needle 7320 to be advanced such that needle 7320 extends through end opening 7340 of head 7308, which can permit needle 7320 to pierce tissue of a subject without interference from head 7308 and/or another component of instrument 7304. In the embodiment shown, needle 7320 can be coupled to seal 7344 to limit the extent to which needle 7320 can extend beyond end opening 7340 of head 7308. In some embodiments, in an advanced configuration, seal 7344 can be configured to seal one or more openings in shaft 7312 and/or head 7308 to, for example, prevent fluid and/or material from entering or exiting shaft 7312 except for fluid and/or material entering or exiting shaft 7312 via tubing 7316 and needle 7320. In some embodiments, in a retracted configuration, seal 7344 can be configured to seal one or more openings in shaft 7312 and/or head 7308 to, for example, encourage fluid and material to enter or exit shaft 7312 through openings 7336 and 7340. In such embodiments, instrument 7304 can be configured to enable an operator to control the manner in which fluid and/or material enter or exit shaft 7312, and shaft 7312 can comprise one or more lengths of tubing (as discussed in detail throughout this disclosure) to enable separation of the various ways in which fluid and/or material can enter or exit shaft 7312. From such an advanced configuration, instrument 7304 is configured to enable at least a portion of needle 7320 and/or tubing 7316 to be retracted into shaft 7312. Such a retracted configuration enables head 7308 to extend beyond needle 7320, which can permit moving fluid into a body of a subject and/or removing fluid and material from a body of a subject without interference from needle 7320. Instrument 7304 is configured to enable advancement and retraction of needle 7320 by an operator (e.g., via an instrument control system, such as that described with respect to instrument control system 126), including an operator of a device, such as a platform for minimally invasive procedures. Various manners in which a needle can be advanced and/or retracted are described in detail below. Tubing 7316 can be configured to advance and retract with needle 7320 to enable needle 7320 and tubing 7316 to remain in fluid communication.

As discussed above, shaft 7312 of instrument 7304 can be coupled to and in fluid communication with a housing such that the housing is in fluid communication with needle 7320. Further, instrument 7304 is configured to be coupled to a fluid reservoir (such as, for example, via tubing 7316 and/or through a housing to which instrument 7304 is coupled) to enable the fluid reservoir to be in fluid communication with shaft 7312 and, therefore, in fluid communication with needle 7320. As with instruments described above, at least a portion of instrument 7304 is configured to be disposed through a trocar (e.g., one of trocars 292, as depicted in FIG. 6) into a body of a subject (e.g., body 288, as depicted in FIG. 6). At least a portion of instrument 7304 is further configured to be coupled to a platform for minimally invasive procedures and controllable by an operator of the platform for minimally invasive procedures (similar to that depicted in FIG. 6), and a platform for minimally invasive procedures (e.g., such as arm 280, as depicted in FIG. 6) can be docked to a trocar (e.g., one or more of trocars 292, as depicted in FIG. 6). If at least a portion of instrument 7304 is disposed through a trocar into a body of a subject, if a fluid reservoir is in fluid communication with shaft 7312, and if shaft 7312 is in fluid communication with needle 7320, instrument 7304 is configured to permit delivery of fluid (e.g., at the direction of an operator) from the fluid reservoir, through shaft 7312, through head 7308, through needle 7320, and through tissue of a subject (as described in detail above). Furthermore, instrument 7304 is configured to permit fluid to be moved into a body of a subject and/or fluid and material to be removed from a body of a subject through head 7308, including, in some configurations, needle 7320 (e.g., at the direction of an operator).

FIG. 24 depicts system 7400 comprising a portion of instrument 7404. In the embodiment shown in FIG. 24, system 7400 comprises head 7408, which is configured to be coupled to shaft 7412 and is depicted coupled to shaft 7412 in the embodiment shown. In FIG. 24, shaft 7412 is depicted with two wavy solid lines representing a break in the depiction of shaft 7412 to indicate that shaft 7412 can extend in the same manner indefinitely between such wavy solid lines and/or can extend at least as long as necessary between such wavy solid lines in order to effect the described embodiments. Therefore, at least some of shaft 7412 is not drawn to scale. Head 7408 can be coupled to shaft 7412 in any suitable way, including in any suitable way to enable movement of head 7408 with respect to shaft 7412, such as, for example, by one or more pins, screws, hinges, adhesive, bolts, and the like. In some embodiments, head 7408 is decouplable from shaft 7412 to enable replacement of head 7408 with a head having similar functionality and/or replacement of head 7408 with a head having different functionality. In other embodiments, head 7408 is unitary with shaft 7412 (e.g., at least partially formed of a single piece of material and, therefore, not couplable to or decouplable from shaft 7412).

In the embodiment shown in FIG. 24, instrument 7404 further comprises tubing 7416 extending through a lumen of shaft 7412. In the embodiment shown, tubing 7416 is configured to be coupled to needle 7420 (and is depicted coupled to needle 7420, in the embodiment shown) to enable shaft 7412 (and, more specifically, tubing 7416 extending through a lumen of shaft 7412) and needle 7420 to be in fluid communication (and are depicted in fluid communication, in the embodiment shown) via tubing 7416. In some embodiments, needle 7420 is in fixed connection with tubing 7416 (such that needle 7420 cannot be decoupled from tubing 7416); and, in other embodiments, needle 7420 is couplable to and decouplable from tubing 7416. Tubing 7416 includes two wavy solid lines depicting breaks in the depiction of tubing 7416 to indicate that tubing 7416 can extend in the same manner indefinitely between such wavy solid lines and/or can extend at least as long as necessary between such wavy solid lines in order to effect the described embodiments. Therefore, at least some of tubing 7416 is not drawn to scale. Tubing 7416 can comprise the same or similar features and characteristics as those depicted and described with respect to tubing 116.

In the embodiment shown in FIG. 24, head 7408 comprises tissue grasper 7424 having first jaw 7428 and second jaw 7432, where at least one of first jaw 7428 and second jaw 7432 are movable to enable grasping and releasing of tissue of a subject. Though tissue grasper 7424 may primarily be used for grasping and releasing tissue, tissue grasper 7424 can grasp, release, and/or otherwise move other items, including suturing devices, instruments, prosthetics, and the like. As discussed above, head 7408 can be coupled to an instrument control system (similar to instrument control system 126) to enable an operator to move head 7408, including moving at least one of first jaw 7428 and second jaw 7432, moving head 7408 with respect to shaft 7412 (as described in detail with respect to other embodiments), and rotating head 7408 with respect to shaft 7412 (as described in detail with respect to other embodiments). For example, in some embodiments, instrument 7404 can be configured to enable movement of first jaw 7428 toward second jaw 7432 such that, if tissue is positioned between first jaw 7428 and second jaw 7432, first jaw 7428 can secure the tissue between first jaw 7428 and second jaw 7432 by applying a force on the tissue in the direction of second jaw 7432. Similarly, in some embodiments, instrument 7404 can be configured to enable movement of second jaw 7432 toward first jaw 7428 such that, if tissue is positioned between first jaw 7428 and second jaw 7432, second jaw 7432 can secure tissue between first jaw 7428 and second jaw 7432 by applying a force on the tissue in the direction of first jaw 7428. As another example, in some embodiments, instrument 7404 can be configured to enable movement of second jaw 7432 toward first jaw 7428 and first jaw 7428 toward second jaw 7432 such that, if tissue is positioned between first jaw 7428 and second jaw 7432, second jaw 7432 and first jaw 7428 can secure the tissue between first jaw 7428 and second jaw 7432 by each applying a force on the tissue in the direction of the other jaw.

In the embodiment shown in FIG. 24, shaft 7312 comprises an opening through which needle extends such that needle 7420 is disposed in fixed relation to shaft 7412 such that needle 7420 moves in substantially the same manner and direction in which head 7408 moves. As depicted in the embodiment shown, needle 7420 is disposed at an angle with respect to shaft 7412. In the embodiment shown, needle 7420 is disposed at a substantially 45 degree angle with respect to shaft 7412; however, in other embodiments, needle 7420 can be disposed at a greater angle (e.g., 50 degrees, 55 degrees, 60 degrees, 65 degrees, 70 degrees, or more) or a lesser angle (e.g., 40 degrees, 35 degrees, 30 degrees, 25 degrees, 20 degrees, or less).

In some embodiments, needle 7420 may extend through the opening in shaft 7412 in a fixed connection such that needle 7420 cannot advance through or retract from the opening in shaft 7412 (e.g., needle 7420 extends a fixed length from shaft 7412). In other embodiments, such as the embodiment shown, instrument 7404 is configured to enable at least a portion of needle 7420 to retract into shaft 7412 and further configured to enable at least a portion of needle 7420 to advance from shaft 7412 (e.g., at a direction of an operator). For example, instrument 7404 is configured to enable at least a portion of needle 7420 to be advanced from shaft 7412 (e.g., via an opening in a portion of shaft 7412). Such an advancement enables needle 7420 to extend a sufficient distance beyond shaft 7412, which can permit needle 7420 to pierce tissue of a subject without interference from instrument 7404. In the embodiment shown, needle 7420 can be coupled to stop 7436 to limit the extent to which needle 7420 can extend from shaft 7412. From such an advanced configuration, instrument 7404 is configured to enable at least a portion of needle 7420 to be retracted into shaft 7412. Such a retracted configuration enables needle 7420 to be retracted into shaft 7412 a sufficient distance to prevent needle 7420 from interfering with any other operations of instrument 7404. Instrument 7404 is configured to enable advancement and retraction of needle 7420 by an operator (e.g., via an instrument control system, such as that described with respect to instrument control system 126), including an operator of a device, such as a platform for minimally invasive procedures. Various manners in which a needle can be advanced and/or retracted are described in detail below. Tubing 7416 can be configured to advance and retract with needle 7420 to enable needle 7420 and tubing 7416 to remain in fluid communication.

As discussed above, shaft 7412 of instrument 7404 can be coupled to and in fluid communication with a housing such that the housing is in fluid communication with needle 7420. Further, instrument 7404 is configured to be coupled to a fluid reservoir (such as, for example, via tubing 7416 and/or through a housing to which instrument 7404 is coupled) to enable the fluid reservoir to be in fluid communication with shaft 7412 and, therefore, in fluid communication with needle 7420. As with instruments described above, at least a portion of instrument 7404 is configured to be disposed through a trocar (e.g., one of trocars 292, as depicted in FIG. 6) into a body of a subject (e.g., body 288, as depicted in FIG. 6). At least a portion of instrument 7404 is further configured to be coupled to a platform for minimally invasive procedures and controllable by an operator of the platform for minimally invasive procedures (similar to that depicted in FIG. 6), and a platform for minimally invasive procedures (e.g., such as arm 280, as depicted in FIG. 6) can be docked to a trocar (e.g., one or more of trocars 292, as depicted in FIG. 6). If at least a portion of instrument 7404 is disposed through a trocar into a body of a subject, if a fluid reservoir is in fluid communication with shaft 7412, and if shaft 7412 is in fluid communication with needle 7420, instrument 7404 is configured to permit delivery of fluid (e.g., at the direction of an operator) from the fluid reservoir, through shaft 7412, through needle 7420, and through tissue of a subject (as described in detail above). Furthermore, instrument 7404 is configured to permit grasping of tissue of a subject with tissue grasper 7424 (e.g., at the direction of an operator).

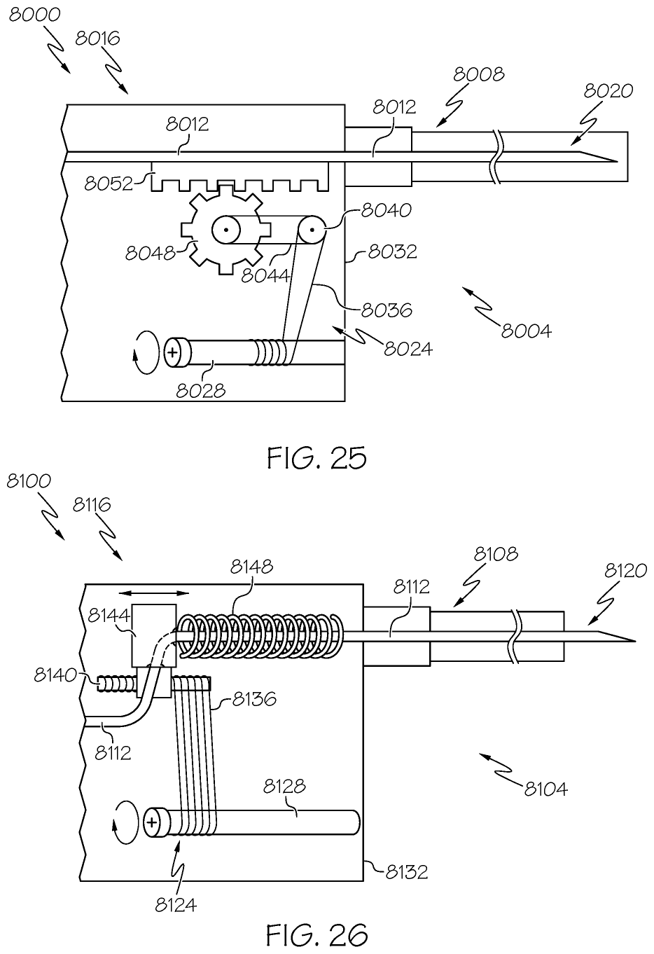

FIGS. 25-32 depict additional examples of the present instruments, systems, methods, and components thereof. As explained above, aspects of any of the embodiments depicted or described above or below may be combined with aspects of the embodiments depicted and described with respect to FIGS. 25-32 to form further embodiments having comparable or different properties and addressing the same or different problems. The embodiments depicted in FIGS. 25-32 and described below can be modified, adapted, and/or supplemented to comprise any of the features shown or described with respect to any of the other systems, instruments, or methods in this disclosure. Similarly, embodiments depicted in other figures or described with respect thereto can be modified, adapted, and/or supplemented to comprise any of the features shown or described with respect to the systems, instruments, and methods of FIGS. 25-32. One or more features of FIGS. 25-32 having the same or similar reference numeral as other features of other embodiments in this disclosure should not be construed to indicate that any feature is limited to the characteristics of another feature having the same or similar reference numeral, or that any feature cannot already have, or cannot be modified to have, features that are different from another feature having the same or similar reference numeral. Though the same or similar feature may have a detailed description with respect to a first embodiment, a second embodiment having the same or similar feature without such a detailed description of such feature (or without any description of such feature) should not be understood to preclude such feature of the second embodiment from comprising the same or similar characteristics as depicted or described with respect to the feature of the first embodiment. For the sake of brevity, all features that an embodiment has or could have may not be described in each separate embodiment.

As discussed above, needles, heads, and systems and components related thereto can be advanced, retracted, or otherwise moved in various ways, some examples of which are depicted in FIGS. 25-32. FIGS. 25-32 and the description related thereto should not be construed to be the only ways in which this disclosure anticipates that the systems and components related thereto can permit advancement, retraction, and other movement of the various components disclosed herein; instead, this disclosure includes some exemplary embodiments that can be considered, while various other configurations that encourage advancement, retraction, and movement of the components disclosed herein are also anticipated. Needles, heads, and systems and components of this disclosure can be advanced, retracted, or otherwise moved in various ways, for example, by an operator, such as an operator of a device, including a platform for minimally invasive procedures, and/or by engaging a portion of the systems of this disclosure (e.g., manually engaging a portion of an instrument that enables control of the instrument). FIGS. 25-32 depict at least a portion of the instruments described in this disclosure and, in particular, various examples of such needles, heads, and systems and components related thereto. Any of the systems and methods of this disclosure can be modified, adapted, and/or supplemented to comprise any such needles, heads, and systems and components related thereto of FIGS. 25-32, in addition to any other needles, heads, and systems and components related thereto described or depicted throughout this disclosure and can operate in the same or similar way as described. In this way, the needles, heads, and systems and components related thereto of FIGS. 25-32 may be used, for example, in minimally invasive procedures as described in this disclosure.

For example, in some embodiments, a system can comprise rigid tubing coupled to (or configured to be coupled to) a needle (e.g., metal tubing, rigid plastic tubing, rigid rubber tubing, and combinations thereof). Rigid tubing can extend through a shaft of an instrument and, in some embodiments, into a housing of an instrument. Rigid tubing can be coupled to an instrument control system, as described herein, and/or accessible by an operator for manual control. To advance a needle in such an embodiment, a force is applied to rigid tubing in the direction of a head of an instrument such that the needle (and any tubing to which it is coupled) advances in substantially the same direction. Similarly, to retract a needle in such an embodiment, a force is applied to rigid tubing in the direction away from a head of an instrument such that the needle advances in substantially the same direction.

FIG. 25 depicts a cross-section of system 8000, which is another example of a manner in which needles, heads, and components related thereto can be advanced, retracted, or otherwise moved. System 8000 comprises a portion of instrument 8004. In the embodiment shown in FIG. 25, system 8000 comprises shaft 8008, which is configured to be coupled to a head of this disclosure in any manner described in this disclosure. In FIG. 25, shaft 8008 is depicted with two wavy solid lines representing a break in the depiction of shaft 8008 to indicate that shaft 8008 can extend in the same manner indefinitely between such wavy solid lines and/or can extend at least as long as necessary between such wavy solid lines in order to effect the described embodiments. Therefore, at least some of shaft 8008 is not drawn to scale.

In the embodiment shown in FIG. 25, instrument 8004 further comprises tubing 8012 extending through a lumen of shaft 8008. Instrument 8004 further comprises a portion of housing 8016, which is coupled to shaft 8008 and through which tubing 8012 extends. In the embodiment shown, tubing 8012 is configured to be coupled to needle 8020 (and is depicted coupled to needle 8020, in the embodiment shown) to enable shaft 8008 (and, more specifically, tubing 8012 extending through a lumen of shaft 8008) and needle 8020 to be in fluid communication (and are depicted in fluid communication, in the embodiment shown) via tubing 8012. In some embodiments, needle 8020 is in fixed connection with tubing 8012 (such that needle 8020 cannot be decoupled from tubing 8012); and, in other embodiments, needle 8020 is couplable to and decouplable from tubing 8020. Tubing 8012 includes two wavy solid lines depicting breaks in the depiction of tubing 8012 to indicate that tubing 8012 can extend in the same manner indefinitely between such wavy solid lines and/or can extend at least as long as necessary between such wavy solid lines in order to effect the described embodiments. Therefore, at least some of tubing 8012 is not drawn to scale. Tubing 8012 can comprise the same or similar features and characteristics as those depicted and described with respect to tubing 116.

In the embodiment shown in FIG. 25, instrument 8004 comprises instrument control system 8024, a portion of which is depicted in FIG. 25. Instrument control system 8024 can be configured to enable an operator to control various components of instrument 8004 and can operate in the same or similar way as described in detail with respect to instrument control system 126. In the embodiment shown instrument control system 8024 comprises control shaft 8028, which extends from and is rotatably coupled to wall 8032 of housing 8016. Control shaft 8028 comprises a control shaft engagement surface (which is not depicted in FIG. 25, but is described in detail with respect to instrument control system 126) to enable a device, such as a platform for minimally invasive procedures, to be coupled to and/or engaged with control shaft 8028. In the embodiment shown in FIG. 25, if a device, such as a platform for minimally invasive procedures, is coupled to and/or engaged with the control shaft engagement surface (and, therefore, coupled to and/or engaged with control shaft 8028), the control shaft engagement surface can be rotated by the device (clockwise and/or counterclockwise) in order to rotate control shaft 8028. For example, if a device, such as a platform for minimally invasive procedures, is coupled to and/or engaged with the control shaft engagement surface (and, therefore, coupled to and/or engaged with control shaft 8028), the control shaft engagement surface can be rotated by the device clockwise (e.g., by an operator) in order to rotate control shaft 8028 clockwise and can be rotated by the device counterclockwise (e.g., by an operator) in order to rotate control shaft 8028 counterclockwise. In some embodiments, the control shaft engagement surface can be rotated by the device clockwise (e.g., by an operator) in order to rotate control shaft 8028 counterclockwise, and vice versa. Control shaft 8028 can be rotated independently via the control shaft engagement surfaces or simultaneously with one or more other control shafts of instrument control system 8024. The device, such as a platform for minimally invasive procedures, can be controllable by an operator of the device to enable the operator to control instrument 8004 via instrument control system 8024.

Instrument control system 8024 further comprises, wire 8036 (which can represent one wire or a system of wires) coupled to control shaft 8028 and engaged with pulley 8040. Instrument control system 8024 further comprises, wire 8044 (which can represent one wire or a system of wires) engaged with pulley 8040 and gear 8048. In the embodiment shown, at least one of tubing 8012 and needle 8020 is coupled to gear engagement surface 8052 (and, in the embodiment shown, a portion of tubing 8012 is coupled to gear engagement surface 8052). Gear 8048 comprises teeth that engage corresponding teeth of gear engagement surface 8052 such that, if gear 8048 is rotated, gear 8048 moves gear engagement surface 8052 and, therefore, moves tubing 8012 and/or needle 8020, at least one of which is coupled (e.g., directly or indirectly) to gear engagement surface 8052.

In the embodiment shown, a device, such as a platform for minimally invasive procedures, and control shaft 8028 can be coupled to gear 8048 to rotate gear 8048 through one or more components. For example, if a device, such as a platform for minimally invasive procedures, is coupled to and/or engaged with the control shaft engagement surface (and, therefore, coupled to and/or engaged with control shaft 8028), the control shaft engagement surface can be rotated clockwise by the device (e.g., by an operator) in order to rotate control shaft 8028 clockwise such that wire 8036 winds about control shaft 8028 to tighten the portion of wire 8036 that engages pulley 8040, which causes gear 8048 to rotate clockwise. As gear 8048 rotates clockwise, teeth of gear 8048 engage with teeth of gear engagement surface 8052 to move gear engagement surface 8052 and components to which it is coupled, such as tubing 8012 and/or needle 8020, toward a head to which instrument 8004 can be coupled. In such a configuration, tubing 8012 and needle 8020 are advanced through shaft 8008 and/or a head to which instrument 8004 can be coupled. Similarly, the control shaft engagement surface can be rotated counterclockwise by the device (e.g., by an operator) in order to rotate control shaft 8028 counterclockwise such that wire 8036 winds about control shaft 8028 to tighten the portion of wire 8036 that engages pulley 8040, which causes gear 8048 to rotate counterclockwise. As gear 8048 rotates counterclockwise, teeth of gear 8048 engage with teeth of gear engagement surface 8052 to move gear engagement surface 8052 and components to which it is coupled, such as tubing 8012 and/or needle 8020, away from a head to which instrument 8004 can be coupled. In such a configuration, tubing 8012 and needle 8020 are retracted through shaft 8008 and/or a head to which instrument 8004 can be coupled.

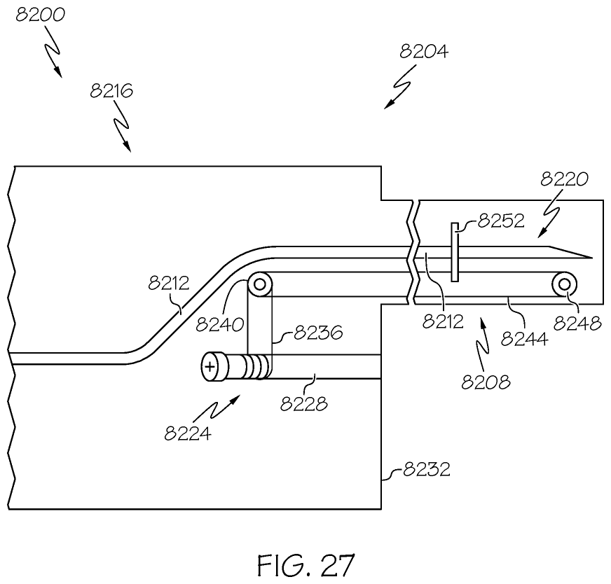

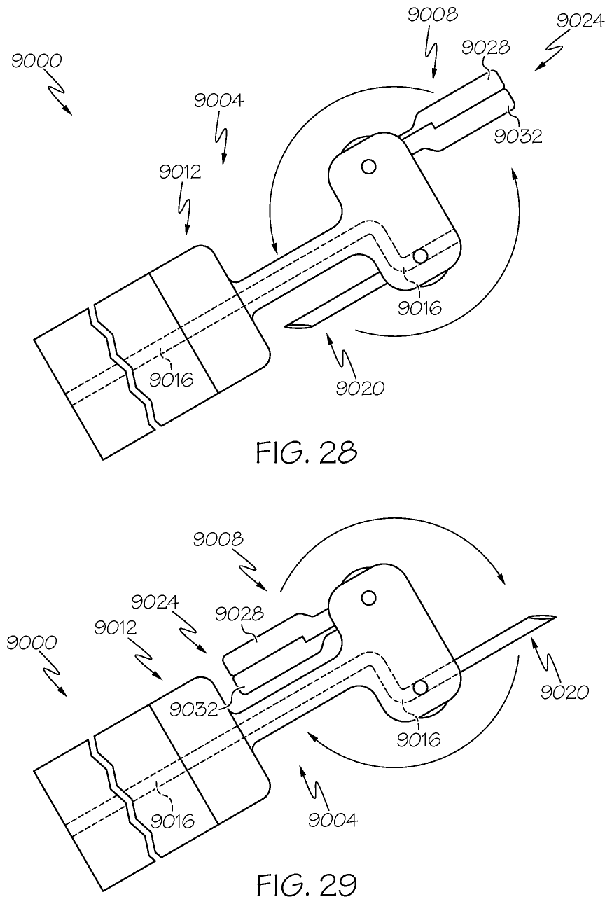

FIG. 26 depicts a cross-section of system 8100, which is another example of a manner in which needles, heads, and components related thereto can be advanced, retracted, or otherwise moved. System 8100 comprises a portion of instrument 8104. In the embodiment shown in FIG. 26, system 8100 comprises shaft 8108, which is configured to be coupled to a head of this disclosure in any manner described in this disclosure. In FIG. 26, shaft 8108 is depicted with two wavy solid lines representing a break in the depiction of shaft 8108 to indicate that shaft 8108 can extend in the same manner indefinitely between such wavy solid lines and/or can extend at least as long as necessary between such wavy solid lines in order to effect the described embodiments. Therefore, at least some of shaft 8108 is not drawn to scale.

In the embodiment shown in FIG. 26, instrument 8104 further comprises tubing 8112 extending through a lumen of shaft 8108. Instrument 8104 further comprises a portion of housing 8116, which is coupled to shaft 8108 and through which tubing 8112 extends. In the embodiment shown, tubing 8112 is configured to be coupled to needle 8120 (and is depicted coupled to needle 8120, in the embodiment shown) to enable shaft 8108 (and, more specifically, tubing 8112 extending through a lumen of shaft 8108) and needle 8120 to be in fluid communication (and are depicted in fluid communication, in the embodiment shown) via tubing 8112. In some embodiments, needle 8120 is in fixed connection with tubing 8112 (such that needle 8120 cannot be decoupled from tubing 8112); and, in other embodiments, needle 8120 is couplable to and decouplable from tubing 8120. Tubing 8112 includes two wavy solid lines depicting breaks in the depiction of tubing 8112 to indicate that tubing 8112 can extend in the same manner indefinitely between such wavy solid lines and/or can extend at least as long as necessary between such wavy solid lines in order to effect the described embodiments. Therefore, at least some of tubing 8112 is not drawn to scale. Tubing 8112 can comprise the same or similar features and characteristics as those depicted and described with respect to tubing 116.

In the embodiment shown in FIG. 26, instrument 8104 comprises instrument control system 8124, a portion of which is depicted in FIG. 26. Instrument control system 8124 can be configured to enable an operator to control various components of instrument 8104 and can operate in the same or similar way as described in detail with respect to instrument control system 126. In the embodiment shown instrument control system 8124 comprises control shaft 8128, which extends from and is rotatably coupled to wall 8132 of housing 8116. Control shaft 8128 comprises a control shaft engagement surface (which is not depicted in FIG. 26, but is described in detail with respect to instrument control system 126) to enable a device, such as a platform for minimally invasive procedures, to be coupled to and/or engaged with control shaft 8128. In the embodiment shown in FIG. 26, if a device, such as a platform for minimally invasive procedures, is coupled to and/or engaged with the control shaft engagement surface (and, therefore, coupled to and/or engaged with control shaft 8128), the control shaft engagement surface can be rotated by the device (clockwise and/or counterclockwise) in order to rotate control shaft 8128. For example, if a device, such as a platform for minimally invasive procedures, is coupled to and/or engaged with the control shaft engagement surface (and, therefore, coupled to and/or engaged with control shaft 8128), the control shaft engagement surface can be rotated by the device clockwise (e.g., by an operator) in order to rotate control shaft 8128 clockwise and can be rotated by the device counterclockwise (e.g., by an operator) in order to rotate control shaft 8128 counterclockwise. In some embodiments, the control shaft engagement surface can be rotated by the device clockwise (e.g., by an operator) in order to rotate control shaft 8128 counterclockwise, and vice versa. Control shaft 8128 can be rotated independently via the control shaft engagement surfaces or simultaneously with one or more other control shafts of instrument control system 8124. The device, such as a platform for minimally invasive procedures, can be controllable by an operator of the device to enable the operator to control instrument 8104 via instrument control system 8124.

Instrument control system 8124 further comprises, wire 8136 (which can represent one wire or a system of wires) coupled to control shaft 8128 and engaged with threaded shaft 8140. In the embodiment shown, tubing 8112 is coupled to shaft interface 8144 and, more specifically, tubing 8112 extends through an interior of shaft interface 8144. Threaded shaft 8140 comprises threads that engage corresponding threads of shaft interface 8144 such that, if threaded shaft 8140 is rotated, shaft interface 8144 moves laterally with respect to threaded shaft 8140, as depicted in FIG. 26, and, therefore, tubing 8012 and/or needle 8020 move laterally with respect to threaded shaft 8140.

In the embodiment shown, if a device, such as a platform for minimally invasive procedures, is coupled to and/or engaged with the control shaft engagement surface (and, therefore, coupled to and/or engaged with control shaft 8128), the control shaft engagement surface can be rotated clockwise by the device (e.g., by an operator) in order to rotate control shaft 8128 clockwise such that wire 8136 winds about control shaft 8128 to tighten the portion of wire 8136 that engages threaded shaft 8140, which causes threaded shaft 8140 to rotate clockwise. As threaded shaft 8140 rotates clockwise, threads of threaded shaft 8140 engage with threads of shaft interface 8144 to move shaft interface 8144 and components to which it is coupled, such as tubing 8112 and/or needle 8120, toward a head to which instrument 8104 can be coupled. In such a configuration, tubing 8112 and needle 8120 are advanced through shaft 8108 and/or a head to which instrument 8104 can be coupled. Instrument 8104 further comprises spring 8148 coupled to shaft interface 8144 and configured to limit the extent to which shaft interface 8144 can advance and, therefore, limit the extent to which needle 8120 can advance from a head to which instrument 8104 is coupled. In other embodiments, spring 8148 can comprise a different component configured to limit the extent to which shaft interface 8148 can advance, such as a threaded shaft. Similarly, the control shaft engagement surface can be rotated counterclockwise by the device (e.g., by an operator) in order to rotate control shaft 8128 counterclockwise such that wire 8136 winds about control shaft 8128 to tighten the portion of wire 8136 that engages threaded shaft 8140, which causes threaded shaft 8140 to rotate counterclockwise. As threaded shaft 8140 rotates counterclockwise, threads of threaded shaft 8140 engage with threads of shaft interface 8144 to move shaft interface 8144 and components to which it is coupled, such as tubing 8112 and/or needle 8120, away from a head to which instrument 8104 can be coupled. In such a configuration, tubing 8112 and needle 8120 are retracted through shaft 8108 and/or a head to which instrument 8104 can be coupled.

FIG. 27 depicts a cross-section of system 8200, which is another example of a manner in which needles, heads, and components related thereto can be advanced, retracted, or otherwise moved. System 8200 comprises a portion of instrument 8204. In the embodiment shown in FIG. 27, system 8200 comprises shaft 8208, which is configured to be coupled to a head of this disclosure in any manner described in this disclosure. In FIG. 27, shaft 8208 is depicted with two wavy solid lines representing a break in the depiction of shaft 8208 to indicate that shaft 8208 can extend in the same manner indefinitely between such wavy solid lines and/or can extend at least as long as necessary between such wavy solid lines in order to effect the described embodiments. Therefore, at least some of shaft 8208 is not drawn to scale.

In the embodiment shown in FIG. 27, instrument 8204 further comprises tubing 8212 extending through a lumen of shaft 8208. Instrument 8204 further comprises a portion of housing 8216, which is coupled to shaft 8208 and through which tubing 8212 extends. In the embodiment shown, tubing 8212 is configured to be coupled to needle 8220 (and is depicted coupled to needle 8220, in the embodiment shown) to enable shaft 8208 (and, more specifically, tubing 8212 extending through a lumen of shaft 8208) and needle 8220 to be in fluid communication (and are depicted in fluid communication, in the embodiment shown) via tubing 8212. In some embodiments, needle 8220 is in fixed connection with tubing 8212 (such that needle 8220 cannot be decoupled from tubing 8212); and, in other embodiments, needle 8220 is couplable to and decouplable from tubing 8220. Tubing 8212 includes two wavy solid lines depicting breaks in the depiction of tubing 8212 to indicate that tubing 8212 can extend in the same manner indefinitely between such wavy solid lines and/or can extend at least as long as necessary between such wavy solid lines in order to effect the described embodiments. Therefore, at least some of tubing 8212 is not drawn to scale. Tubing 8212 can comprise the same or similar features and characteristics as those depicted and described with respect to tubing 116.

In the embodiment shown in FIG. 27, instrument 8204 comprises instrument control system 8224, a portion of which is depicted in FIG. 27. Instrument control system 8224 can be configured to enable an operator to control various components of instrument 8204 and can operate in the same or similar way as described in detail with respect to instrument control system 126. In the embodiment shown instrument control system 8224 comprises control shaft 8228, which extends from and is rotatably coupled to wall 8232 of housing 8216. Control shaft 8228 comprises a control shaft engagement surface (which is not depicted in FIG. 27, but is described in detail with respect to instrument control system 126) to enable a device, such as a platform for minimally invasive procedures, to be coupled to and/or engaged with control shaft 8228. In the embodiment shown in FIG. 27, if a device, such as a platform for minimally invasive procedures, is coupled to and/or engaged with the control shaft engagement surface (and, therefore, coupled to and/or engaged with control shaft 8228), the control shaft engagement surface can be rotated by the device (clockwise and/or counterclockwise) in order to rotate control shaft 8228. For example, if a device, such as a platform for minimally invasive procedures, is coupled to and/or engaged with the control shaft engagement surface (and, therefore, coupled to and/or engaged with control shaft 8228), the control shaft engagement surface can be rotated by the device clockwise (e.g., by an operator) in order to rotate control shaft 8228 clockwise and can be rotated by the device counterclockwise (e.g., by an operator) in order to rotate control shaft 8228 counterclockwise. In some embodiments, the control shaft engagement surface can be rotated by the device clockwise (e.g., by an operator) in order to rotate control shaft 8228 counterclockwise, and vice versa. Control shaft 8228 can be rotated independently via the control shaft engagement surfaces or simultaneously with one or more other control shafts of instrument control system 8224. The device, such as a platform for minimally invasive procedures, can be controllable by an operator of the device to enable the operator to control instrument 8204 via instrument control system 8224.

Instrument control system 8224 further comprises, wire 8236 (which can represent one wire or a system of wires) coupled to control shaft 8228 and engaged with pulley 8240. Instrument control system 8224 further comprises, wire 8244 (which can represent one wire or a system of wires) engaged with pulley 8240 and pulley 8248. In the embodiment shown, at least one of tubing 8212 and needle 8220 is coupled to stop 8252, and stop 8252 is coupled to wire 8244 such that if wire 8244 moves, stop 8252 (and, therefore, tubing 8212 and needle 8220) moves with wire 8244.

In the embodiment shown, if a device, such as a platform for minimally invasive procedures, is coupled to and/or engaged with the control shaft engagement surface (and, therefore, coupled to and/or engaged with control shaft 8228), the control shaft engagement surface can be rotated clockwise by the device (e.g., by an operator) in order to rotate control shaft 8228 clockwise such that wire 8236 winds about control shaft 8228 to tighten the portion of wire 8236 that engages pulley 8240, which causes wire 8244 to rotate clockwise about pulley 8248. As wire 8244 rotates clockwise about pulley 8248, wire 8244 moves stop 8252 and components to which it is coupled, such as tubing 8212 and/or needle 8220, toward a head to which instrument 8204 can be coupled. In such a configuration, tubing 8212 and needle 8220 are advanced through shaft 8208 and/or a head to which instrument 8204 can be coupled. Similarly, the control shaft engagement surface can be rotated counterclockwise by the device (e.g., by an operator) in order to rotate control shaft 8228 counterclockwise such that wire 8236 winds about control shaft 8228 to tighten the portion of wire 8236 that engages pulley 8240, which causes wire 8244 to rotate counterclockwise about pulley 8248. As wire 8244 rotates counterclockwise about pulley 8248, wire 8244 moves stop 8252 and components to which it is coupled, such as tubing 8212 and/or needle 8220, away from a head to which instrument 8204 can be coupled. In such a configuration, tubing 8212 and needle 8220 are retracted through shaft 8208 and/or a head to which instrument 8204 can be coupled.

FIGS. 28-31 depict other examples of a manner in which needles, heads, and components related thereto can be advanced, retracted, or otherwise moved. Any of the below heads can comprise the same or similar features or be coupled to the same or similar components as those heads described with respect to other figures. For example, the heads described below can be movable and rotatable in the same way as the head described above. FIGS. 28-29 depict system 9000 comprising a portion of instrument 9004. In the embodiment shown in FIGS. 28-29, system 9000 comprises head 9008, which is configured to be coupled to shaft 9012 and is depicted coupled to shaft 9012 in the embodiment shown. In FIGS. 28-29, shaft 9012 is depicted with two wavy solid lines representing a break in the depiction of shaft 9012 to indicate that shaft 9012 can extend in the same manner indefinitely between such wavy solid lines and/or can extend at least as long as necessary between such wavy solid lines in order to effect the described embodiments. Therefore, at least some of shaft 9012 is not drawn to scale. Head 9008 can be coupled to shaft 9012 in any suitable way, including in any suitable way to enable movement of head 9008 with respect to shaft 9012, such as, for example, by one or more pins, screws, hinges, adhesive, bolts, and the like. In some embodiments, head 9008 is decouplable from shaft 9012 to enable replacement of head 9008 with a head having similar functionality and/or replacement of head 9008 with a head having different functionality. In other embodiments, head 9008 is unitary with shaft 9012 (e.g., at least partially formed of a single piece of material and, therefore, not couplable to or decouplable from shaft 9012).

In the embodiment shown in FIGS. 28-29, instrument 9004 further comprises tubing 9016 extending through a lumen of shaft 9012. In the embodiment shown, tubing 9016 is configured to be coupled to needle 9020 (and is depicted decoupled from needle 9020, in the embodiment shown in FIG. 28, and is depicted coupled to needle 9020, in the embodiment shown in FIG. 29) to enable shaft 9012 (and, more specifically, tubing 9016 extending through a lumen of shaft 9012) and needle 9020 to be in fluid communication (and are depicted in fluid communication, in the embodiment shown in FIG. 29) via tubing 9016. In other embodiments, tubing 9016 is coupled to and remains coupled to needle 9020 during advancement and retraction of needle 9020. Tubing 9016 includes two wavy solid lines depicting breaks in the depiction of tubing 9016 to indicate that tubing 9016 can extend in the same manner indefinitely between such wavy solid lines and/or can extend at least as long as necessary between such wavy solid lines in order to effect the described embodiments. Therefore, at least some of tubing 9016 is not drawn to scale. Tubing 9016 can comprise the same or similar features and characteristics as those depicted and described with respect to tubing 116.

In the embodiment shown in FIGS. 28-29, head 9008 comprises suture grasper 9024 having first jaw 9028 and second jaw 9032, where at least one of first jaw 9028 and second jaw 9032 are movable to enable grasping and releasing of a device for suturing tissue of a subject—a “suturing device” (e.g., such as a needle coupled to suture). Though suture grasper 9024 may primarily be used for grasping and releasing suturing devices, suture grasper 9024 can grasp, release, and/or otherwise move other items, including tissue, instruments, prosthetics, and the like. As discussed above, head 9008 can be coupled to an instrument control system (similar to instrument control system 126) to enable an operator to move head 9008, including moving at least one of first jaw 9028 and second jaw 9032, moving head 9008 with respect to shaft 9012 (as described in detail with respect to other embodiments), and rotating head 9008 with respect to shaft 9012 (as described in detail with respect to other embodiments). For example, in some embodiments, instrument 9004 can be configured to enable movement of first jaw 9028 toward second jaw 9032 such that, if a suturing device is positioned between first jaw 9028 and second jaw 9032, first jaw 9028 can secure the suturing device between first jaw 9028 and second jaw 9032 by applying a force on the suturing device in the direction of second jaw 9032 to enable an operator of instrument 9004 to suture a patient with the suturing device. Similarly, in some embodiments, instrument 9004 can be configured to enable movement of second jaw 9032 toward first jaw 9028 such that, if a suturing device is positioned between first jaw 9028 and second jaw 9032, second jaw 9032 can secure the suturing device between first jaw 9028 and second jaw 9032 by applying a force on the suturing device in the direction of first jaw 9028 to enable an operator of instrument 9004 to suture a patient with the suturing device. As another example, in some embodiments, instrument 9004 can be configured to enable movement of second jaw 9032 toward first jaw 9028 and first jaw 9028 toward second jaw 9032 such that, if a suturing device is positioned between first jaw 9028 and second jaw 9032, second jaw 9032 and first jaw 9028 can secure the suturing device between first jaw 9028 and second jaw 9032 by each applying a force on the suturing device in the direction of the other jaw to enable an operator of instrument 9004 to suture a patient with the suturing device.

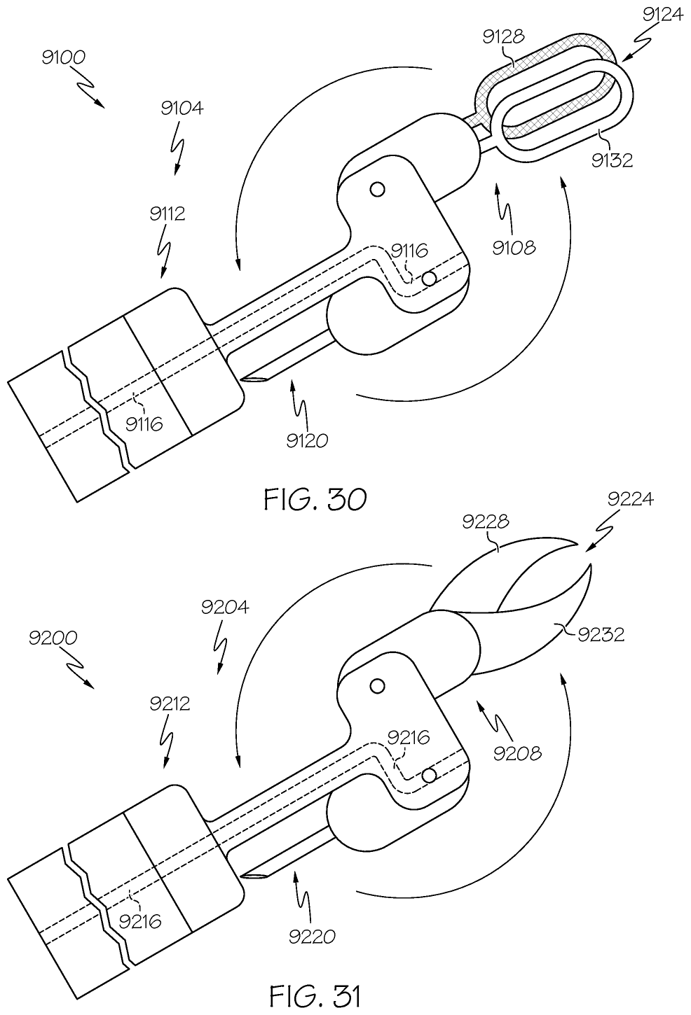

FIG. 30 depicts system 9100 comprising a portion of instrument 9104. In the embodiment shown in FIG. 30, system 9100 comprises head 9108, which is configured to be coupled to shaft 9112 and is depicted coupled to shaft 9112 in the embodiment shown. In FIG. 30, shaft 9112 is depicted with two wavy solid lines representing a break in the depiction of shaft 9112 to indicate that shaft 9112 can extend in the same manner indefinitely between such wavy solid lines and/or can extend at least as long as necessary between such wavy solid lines in order to effect the described embodiments. Therefore, at least some of shaft 9112 is not drawn to scale. Head 9108 can be coupled to shaft 9112 in any suitable way, including in any suitable way to enable movement of head 9108 with respect to shaft 9112, such as, for example, by one or more pins, screws, hinges, adhesive, bolts, and the like. In some embodiments, head 9108 is decouplable from shaft 9112 to enable replacement of head 9108 with a head having similar functionality and/or replacement of head 9108 with a head having different functionality. In other embodiments, head 9108 is unitary with shaft 9112 (e.g., at least partially formed of a single piece of material and, therefore, not couplable to or decouplable from shaft 9112).

In the embodiment shown in FIG. 30, instrument 9104 further comprises tubing 9116 extending through a lumen of shaft 9112. In the embodiment shown, tubing 9116 is configured to be coupled to needle 9120 (and is depicted decoupled from needle 9120, in the embodiment shown in FIG. 30) to enable shaft 9112 (and, more specifically, tubing 9116 extending through a lumen of shaft 9112) and needle 9120 to be in fluid communication via tubing 9116. In other embodiments, tubing 9116 is coupled to and remains coupled to needle 9020 during advancement and retraction of needle 9120. Tubing 9116 includes two wavy solid lines depicting breaks in the depiction of tubing 9116 to indicate that tubing 9116 can extend in the same manner indefinitely between such wavy solid lines and/or can extend at least as long as necessary between such wavy solid lines in order to effect the described embodiments. Therefore, at least some of tubing 9116 is not drawn to scale. Tubing 9116 can comprise the same or similar features and characteristics as those depicted and described with respect to tubing 116.

In the embodiment shown in FIG. 30, head 9108 comprises tissue grasper 9124 having first jaw 9128 and second jaw 9132, where at least one of first jaw 9128 and second jaw 9132 are movable to enable grasping and releasing of tissue of a subject. Though tissue grasper 9124 may primarily be used for grasping and releasing tissue, tissue grasper 9124 can grasp, release, and/or otherwise move other items, including suturing devices, instruments, prosthetics, and the like. As discussed above, head 9108 can be coupled to an instrument control system (similar to instrument control system 126) to enable an operator to move head 9108, including moving at least one of first jaw 9128 and second jaw 9132, moving head 9108 with respect to shaft 9112 (as described in detail with respect to other embodiments), and rotating head 9108 with respect to shaft 9112 (as described in detail with respect to other embodiments). For example, in some embodiments, instrument 9104 can be configured to enable movement of first jaw 9128 toward second jaw 9132 such that, if tissue is positioned between first jaw 9128 and second jaw 9132, first jaw 9128 can secure the tissue between first jaw 9128 and second jaw 9132 by applying a force on the tissue in the direction of second jaw 9132. Similarly, in some embodiments, instrument 9104 can be configured to enable movement of second jaw 9132 toward first jaw 9128 such that, if tissue is positioned between first jaw 9128 and second jaw 9132, second jaw 9132 can secure the tissue between first jaw 9128 and second jaw 9132 by applying a force on the tissue in the direction of first jaw 9128. As another example, in some embodiments, instrument 9104 can be configured to enable movement of second jaw 9132 toward first jaw 9128 and first jaw 9128 toward second jaw 9132 such that, if tissue is positioned between first jaw 9128 and second jaw 9132, second jaw 9132 and first jaw 9128 can secure the tissue between first jaw 9128 and second jaw 9132 by each applying a force on the tissue in the direction of the other jaw.

FIG. 31 depicts system 9200 comprising a portion of instrument 9204. In the embodiment shown in FIG. 31, system 9200 comprises head 9208, which is configured to be coupled to shaft 9212 and is depicted coupled to shaft 9212 in the embodiment shown. In FIG. 31, shaft 9212 is depicted with two wavy solid lines representing a break in the depiction of shaft 9212 to indicate that shaft 9212 can extend in the same manner indefinitely between such wavy solid lines and/or can extend at least as long as necessary between such wavy solid lines in order to effect the described embodiments. Therefore, at least some of shaft 9212 is not drawn to scale. Head 9208 can be coupled to shaft 9212 in any suitable way, including in any suitable way to enable movement of head 9208 with respect to shaft 9212, such as, for example, by one or more pins, screws, hinges, adhesive, bolts, and the like. In some embodiments, head 9208 is decouplable from shaft 9212 to enable replacement of head 9208 with a head having similar functionality and/or replacement of head 9208 with a head having different functionality. In other embodiments, head 9208 is unitary with shaft 9212 (e.g., at least partially formed of a single piece of material and, therefore, not couplable to or decouplable from shaft 9212).

In the embodiment shown in FIG. 31, instrument 9204 further comprises tubing 9216 extending through a lumen of shaft 9212. In the embodiment shown, tubing 9216 is configured to be coupled to needle 9220 (and is depicted decoupled from needle 9220, in the embodiment shown in FIG. 31) to enable shaft 9212 (and, more specifically, tubing 9216 extending through a lumen of shaft 9212) and needle 9220 to be in fluid communication via tubing 9216. In other embodiments, tubing 9216 is coupled to and remains coupled to needle 9020 during advancement and retraction of needle 9220. Tubing 9216 includes two wavy solid lines depicting breaks in the depiction of tubing 9216 to indicate that tubing 9216 can extend in the same manner indefinitely between such wavy solid lines and/or can extend at least as long as necessary between such wavy solid lines in order to effect the described embodiments. Therefore, at least some of tubing 9216 is not drawn to scale. Tubing 9216 can comprise the same or similar features and characteristics as those depicted and described with respect to tubing 116.

In the embodiment shown in FIG. 31, head 9208 comprises tissue cutting device 9224 having first blade 9228 and second blade 9232, where at least one of first blade 9228 and second blade 9232 are movable to enable cutting of tissue of a subject. As discussed above, head 9208 can be coupled to an instrument control system (similar to instrument control system 126) to enable an operator to move head 9208, including moving at least one of first blade 9228 and second blade 9232, moving head 9208 with respect to shaft 9212 (as described in detail with respect to other embodiments), and rotating head 9208 with respect to shaft 9212 (as described in detail with respect to other embodiments). For example, in some embodiments, instrument 9204 can be configured to enable movement of first blade 9228 toward second blade 9232 such that, if tissue is positioned between first blade 9228 and second blade 9232, first blade 9228 and second blade 9232 can cut the tissue by applying a force on the tissue in the direction of second blade 9232. Similarly, in some embodiments, instrument 9204 can be configured to enable movement of second blade 9232 toward first blade 9228 such that, if tissue is positioned between first blade 9228 and second blade 9232, first blade 9228 and second blade 9232 can cut the tissue between by applying a force on the tissue in the direction of first blade 9228. As another example, in some embodiments, instrument 9204 can be configured to enable movement of second blade 9232 toward first blade 9228 and first blade 9228 toward second blade 9232 such that, if tissue is positioned between first blade 9228 and second blade 9232, second blade 9232 and first blade 9228 can cut the tissue between first blade 9228 and second blade 9232 by each applying a force on the tissue in the direction of the other blade.

In the embodiments shown in FIGS. 28-31, each instrument is configured to move the needle and the head when activated by an operator. The instruments and/or the device to which the instruments are coupled (such as a platform for minimally invasive procedures) can enable an operator to move the needle and the head simultaneously (e.g., such that each of the head and the needle are in motion at substantially the same time) or independently (e.g., such that one of the head and the needle are stationary while the other of the head and the needle is in motion, and vice versa). For example, if the head extends away from the shaft and the needle extends toward the shaft and is not in fluid communication with the shaft, the operator can activate the instrument to move the needle and head such that the needle extends away from the shaft and the head extends toward the shaft. In some embodiments, the needle is decouplable from and couplable to tubing such that, if an operator activates the instrument to move the needle to extend away from the shaft and the head to extend toward the shaft, the needle and the tubing are coupled and in fluid communication. In other embodiments, the needle is coupled to and remains coupled to the tubing through any movement of the needle. Similarly, if the head extends toward the shaft and the needle extends away from the shaft, the operator can activate the instrument to move the needle and head such that the needle extends toward the shaft and the head extends away from the shaft. Any of the instruments in this disclosure can be configured in such a way, for example, by coupling a head and a needle to an instrument control system to enable the instrument control system to move the head and the needle (e.g., with springs, pins, adhesive, bolts, and the like).

For example, in the embodiment shown in FIGS. 28-29, instrument 9004 is configured to move needle 9020 and head 9008 when activated by an operator (e.g., such as an operator of a platform for minimally invasive procedures) such that, if head 9008 extends away from shaft 9012 and needle 9020 extends toward shaft 9012 and is not in fluid communication with shaft 9012, the operator can activate instrument 9004 to move needle 9020 and head 9008 (e.g., via an instrument control system) such that needle 9020 extends away from shaft 9012 and is in fluid communication with shaft 9012 and head 9008 extends toward shaft 9012. As discussed above, in some embodiments, needle 9020 and shaft 9012 (and, more specifically, tubing within shaft 9012) can remain in fluid communication throughout movement of needle 9020. Such a configuration can permit needle 9020 to pierce tissue of a subject without interference from head 9008 and/or another component of instrument 9004. Similarly, if head 9008 extends toward shaft 9012 and needle 9020 extends away from shaft 9012 and is in fluid communication with shaft 9012, the operator can activate instrument 9004 to move needle 9020 and head 9008 (e.g., via an instrument control system) such that needle 9020 extends toward shaft 9012 and is not in fluid communication with shaft 9012 and head 9008 extends away from shaft 9012. Such a configuration can permit suturing of a subject with suture grasper 9024 without interference from needle 9020.

As another example, in the embodiment shown in FIG. 30, instrument 9104 is configured to move needle 9120 and head 9108 when activated by an operator (e.g., such as an operator of a platform for minimally invasive procedures) such that, if head 9108 extends away from shaft 9112 and needle 9120 extends toward shaft 9112 and is not in fluid communication with shaft 9112, the operator can activate instrument 9104 to move needle 9120 and head 9108 (e.g., via an instrument control system) such that needle 9120 extends away from shaft 9112 and is in fluid communication with shaft 9112 and head 9108 extends toward shaft 9112. As discussed above, in some embodiments, needle 9120 and shaft 9112 (and, more specifically, tubing within shaft 9112) can remain in fluid communication throughout movement of needle 9120. Such a configuration can permit needle 9120 to pierce tissue of a subject without interference from head 9108 and/or another component of instrument 9104. Similarly, if head 9108 extends toward shaft 9112 and needle 9120 extends away from shaft 9112 and is in fluid communication with shaft 9112, the operator can activate instrument 9104 to move needle 9120 and head 9108 (e.g., via an instrument control system) such that needle 9120 extends toward shaft 9112 and is not in fluid communication with shaft 9112 and head 9108 extends away from shaft 9112. Such a configuration can permit grasping of tissue of a subject with tissue grasper 9124 without interference from needle 9120.

As still another example, in the embodiment shown in FIG. 31, instrument 9204 is configured to move needle 9220 and head 9208 when activated by an operator (e.g., such as an operator of a platform for minimally invasive procedures) such that, if head 9208 extends away from shaft 9212 and needle 9220 extends toward shaft 9212 and is not in fluid communication with shaft 9212, the operator can activate instrument 9204 to move needle 9220 and head 9208 (e.g., via an instrument control system) such that needle 9220 extends away from shaft 9212 and is in fluid communication with shaft 9212 and head 9208 extends toward shaft 9212. As discussed above, in some embodiments, needle 9220 and shaft 9212 (and, more specifically, tubing within shaft 9212) can remain in fluid communication throughout movement of needle 9220. Such a configuration can permit needle 9220 to pierce tissue of a subject without interference from head 9208 and/or another component of instrument 9204. Similarly, if head 9208 extends toward shaft 9212 and needle 9220 extends away from shaft 9212 and is in fluid communication with shaft 9212, the operator can activate instrument 9204 to move needle 9220 and head 9208 (e.g., via an instrument control system) such that needle 9220 extends toward shaft 9212 and is not in fluid communication with shaft 9212 and head 9208 extends away from shaft 9212. Such a configuration can permit cutting of tissue of a subject with tissue cutting device 9224 without interference from needle 9220.

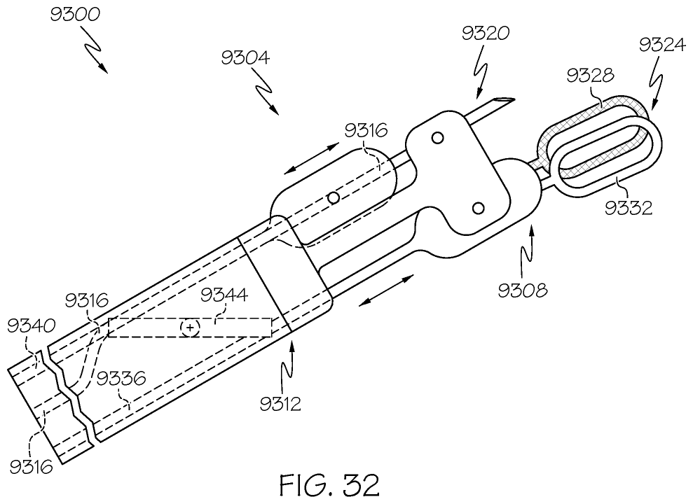

FIG. 32 depicts other examples of a manner in which needles, heads, and components related thereto can be advanced, retracted, or otherwise moved. The below head can comprise the same or similar features or be coupled to the same or similar components as those heads described with respect to other figures. For example, the head described below can be movable and rotatable in the same way as the head described above. FIG. 32 depicts system 9300 comprising a portion of instrument 9304. In the embodiment shown in FIG. 32, system 9300 comprises head 9308, which is configured to be coupled to shaft 9312 and is depicted coupled to shaft 9312 in the embodiment shown. In FIG. 32, shaft 9312 is depicted with two wavy solid lines representing a break in the depiction of shaft 9312 to indicate that shaft 9312 can extend in the same manner indefinitely between such wavy solid lines and/or can extend at least as long as necessary between such wavy solid lines in order to effect the described embodiments. Therefore, at least some of shaft 9312 is not drawn to scale. Head 9308 can be coupled to shaft 9312 in any suitable way, including in any suitable way to enable movement of head 9308 with respect to shaft 9312, such as, for example, by one or more pins, screws, hinges, adhesive, bolts, and the like. In some embodiments, head 9308 is decouplable from shaft 9312 to enable replacement of head 9308 with a head having similar functionality and/or replacement of head 9308 with a head having different functionality. In other embodiments, head 9308 is unitary with shaft 9312 (e.g., at least partially formed of a single piece of material and, therefore, not couplable to or decouplable from shaft 9312).

In the embodiment shown in FIG. 32, instrument 9304 further comprises tubing 9316 extending through a lumen of shaft 9312. In the embodiment shown, tubing 9316 is configured to be coupled to needle 9320 (and is depicted coupled to needle 9320, in the embodiment shown in FIG. 32) to enable shaft 9312 (and, more specifically, tubing 9316 extending through a lumen of shaft 9312) and needle 9320 to be in fluid communication via tubing 9316. Tubing 9316 includes two wavy solid lines depicting breaks in the depiction of tubing 9316 to indicate that tubing 9316 can extend in the same manner indefinitely between such wavy solid lines and/or can extend at least as long as necessary between such wavy solid lines in order to effect the described embodiments. Therefore, at least some of tubing 9316 is not drawn to scale. Tubing 9316 can comprise the same or similar features and characteristics as those depicted and described with respect to tubing 116.

In the embodiment shown in FIG. 32, head 9308 comprises tissue grasper 9324 having first jaw 9328 and second jaw 9332, where at least one of first jaw 9328 and second jaw 9332 are movable to enable grasping and releasing of tissue of a subject. Though tissue grasper 9324 may primarily be used for grasping and releasing tissue, tissue grasper 9324 can grasp, release, and/or otherwise move other items, including suturing devices, instruments, prosthetics, and the like. As discussed above, head 9308 can be coupled to an instrument control system (similar to instrument control system 126) to enable an operator to move head 9308, including moving at least one of first jaw 9328 and second jaw 9332, moving head 9308 with respect to shaft 9312 (as described in detail with respect to other embodiments), and rotating head 9308 with respect to shaft 9312 (as described in detail with respect to other embodiments). For example, in some embodiments, instrument 9304 can be configured to enable movement of first jaw 9328 toward second jaw 9332 such that, if tissue is positioned between first jaw 9328 and second jaw 9332, first jaw 9328 can secure the tissue between first jaw 9328 and second jaw 9332 by applying a force on the tissue in the direction of second jaw 9332. Similarly, in some embodiments, instrument 9304 can be configured to enable movement of second jaw 9332 toward first jaw 9328 such that, if tissue is positioned between first jaw 9328 and second jaw 9332, second jaw 9332 can secure the tissue between first jaw 9328 and second jaw 9332 by applying a force on the tissue in the direction of first jaw 9328. As another example, in some embodiments, instrument 9304 can be configured to enable movement of second jaw 9332 toward first jaw 9328 and first jaw 9328 toward second jaw 9332 such that, if tissue is positioned between first jaw 9328 and second jaw 9332, second jaw 9332 and first jaw 9328 can secure the tissue between first jaw 9328 and second jaw 9332 by each applying a force on the tissue in the direction of the other jaw.

System 9300 of FIG. 32 further comprises rod 9336 coupled to head 9008 and rod 9340 coupled to needle 9320. Further, rod 9336 is coupled to rod 9340 by connecting device 9344 (which is not drawn to scale). Instrument 9304 is configured to move needle 9320 and head 9308 when activated by an operator (e.g., such as an operator of a platform for minimally invasive procedures) such that, if head 9308 extends further from shaft 9312 than needle 9320 (such as in the embodiment shown in FIG. 32), the operator can activate instrument 9304 to move needle 9320 or head 9308 by moving rod 9340 and/or rod 9336 (e.g., via an instrument control system) such that needle 9320 and tubing 9316 is advanced to extend further from shaft 9312 than head 9308, and head 9308 is retracted such that needle 9320 extends further from shaft 9312 than head 9308. In other embodiments, rod 9340 and/or rod 9336 can be moved through movement of connecting device 9344, which can be controllable by an operator via one or more components of an instrument control system as described throughout this disclosure. Such a configuration can permit needle 9320 to pierce tissue of a subject without interference from head 9308 and/or another component of instrument 9304. Similarly, if needle 9320 extends further from shaft 9312 than head 9308, the operator can activate instrument 9304 to move needle 9320 or head 9308 by moving rod 9340 and/or rod 9336 (e.g., via an instrument control system) such that head 9308 is advanced to extend further from shaft 9312 than needle 9320, and needle 9320 is retracted such that head 9308 extends further from shaft 9312 than needle 9320. Such a configuration can permit grasping of tissue of a subject with tissue grasping 9324 without interference from needle 9320.

This present disclosure further includes various methods for medical procedures, including methods for minimally invasive medical procedures, such as those that relate to use of the systems and instruments of the present disclosure. For example, in some embodiments, the methods include creating at least one incision in a body of a subject; disposing at least a portion of a trocar within the at least one incision to provide access to a target area within the body of the subject; disposing at least a portion of an instrument within the trocar such that the instrument can access the target area within the body of the subject, where the instrument comprises a head; and a shaft in fluid communication with a fluid reservoir, where the fluid reservoir comprises fluid, and at least a portion of the fluid in the fluid reservoir is medication (or serves a medical benefit or purpose); where the instrument is configured to permit delivery of fluid from the fluid reservoir, through the shaft, and through tissue of the subject; delivering fluid from the fluid reservoir, through the shaft, and through tissue of the target area with the instrument. In some embodiments, the method further comprises coupling the instrument to a platform for minimally invasive procedure to enable an operator to control the instrument. In some embodiments, the instrument further comprises a needle in fluid communication with the shaft of the instrument, and where prior to delivering fluid from the fluid reservoir, the method further comprises piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument. In some embodiments, the needle of the instrument is movable with the respect to the shaft, where prior to piercing a surface layer of tissue of the subject with the needle, the method further comprises moving the needle of the instrument to position the needle to access the target area. In some embodiments, prior to piercing a surface layer of tissue of the subject with the needle, the method further comprises coupling a needle to at least one of the shaft and the head of the instrument. In some embodiments, the instrument further comprises a pump configured to pump fluid from the fluid reservoir and through the shaft, and where prior to delivering fluid from the fluid reservoir, the method comprises activating the pump using the platform for minimally invasive procedures to pump fluid from the fluid reservoir and through the shaft.

In some embodiments, the method further comprises continuing to pierce a surface layer of tissue of the target area of the subject with the needle to enable fluid from the fluid reservoir to be delivered through tissue of the target area with the instrument; continuing to deliver fluid from the fluid reservoir, through the shaft, through the needle, and through tissue of the target area with the instrument; determining that sufficient fluid has been delivered to the target area of the subject; and suturing a mesh (e.g., where and when appropriate) to the target area of the subject. In some embodiments, prior to suturing a mesh to the target area of the subject, the method further comprises the option of suturing a defect in the target area of the subject. In some embodiments, at least a portion of the fluid in the fluid reservoir is fluid that is detectable within the body with a viewing device, and determining that sufficient fluid has been delivered to the target area of the subject comprises observing the target area of the subject with a viewing device; and determining that sufficient fluid is detectable within the target area. In some embodiments, the instrument further comprises a needle in fluid communication with the shaft of the instrument, and where prior to delivering fluid from the fluid reservoir, the method further comprises suturing a mesh to the target area of the subject; piercing a surface layer of tissue of the target area of the subject with the needle to enable fluid from the fluid reservoir to be delivered through tissue of the target area with the instrument; delivering fluid from the fluid reservoir, through the shaft, through the needle, and through tissue of the target area with the instrument; continuing to pierce a surface layer of tissue of the target area of the subject with the needle to enable fluid from the fluid reservoir to be delivered through tissue of the target area with the instrument; continuing to deliver fluid from the fluid reservoir, through the shaft, through the needle, and through tissue of the target area with the instrument; and determining that sufficient fluid has been delivered to the target area of the subject. In some embodiments, prior to suturing a mesh to the target area of the subject, the method further comprises the option of suturing a defect in the target area of the subject. In some embodiments, at least a portion of the fluid in the fluid reservoir is fluid that is detectable within the body with a viewing device, and determining that sufficient fluid has been delivered to the target area of the subject comprises observing the target area of the subject with a viewing device; and determining that sufficient fluid is detectable within the target area.

In some embodiments, the head comprises a tissue grasper having a first jaw and a second jaw, where at least one of the first jaw and the second jaw are movable to enable grasping and releasing of tissue of a subject, and the instrument further comprises a needle in fluid communication with the shaft of the instrument, where prior to delivering fluid from the fluid reservoir, the method further comprises grasping tissue with the tissue grasper to assist in accessing the target area; piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument. In some embodiments, the head comprises a suture grasper having a first jaw and a second jaw, where at least one of the first jaw and the second jaw are movable to enable grasping and releasing of a device for suturing tissue of a subject, and the instrument further comprises a needle in fluid communication with the shaft of the instrument, where prior to delivering fluid from the fluid reservoir, the method further comprises suturing a mesh to the target area of the subject with the suture grasper; piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument. In some embodiments, prior to suturing a mesh to the target area of the subject with the suture grasper, the method further comprises the option of suturing a defect in the target area of the subject with the suture grasper. In some embodiments, the head comprises a suture grasper having a first jaw and a second jaw, where at least one of the first jaw and the second jaw are movable to enable grasping and releasing of a device for suturing tissue of a subject, and the instrument further comprises a needle in fluid communication with the shaft of the instrument, where prior to delivering fluid from the fluid reservoir, the method further comprises piercing a surface layer of tissue of the target area of the subject with the needle to enable fluid from the fluid reservoir to be delivered through tissue of the target area with the instrument; delivering fluid from the fluid reservoir, through the shaft, through the needle, and through tissue of the target area with the instrument; and suturing a mesh (where and when appropriate) to the target area of the subject with the suture grasper. In some embodiments, prior to suturing a mesh to the target area of the subject with the suture grasper, the method further comprises the option of suturing a defect in the target area of the subject with the suture grasper. In some embodiments, the head comprises a tissue cutting device having a first blade and a second blade, where at least one of the first blade and the second blade are movable to enable cutting of the tissue of a subject, and the instrument further comprises a needle in fluid communication with the shaft of the instrument, where prior to delivering fluid from the fluid reservoir, the method further comprises cutting tissue with the tissue cutting device to assist in accessing the target area; piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument. In some embodiments, the head is at least one of configured to enable fluid and material to be removed from the body of a subject through the head by a suction device; and configured to enable fluid to move into the body of a subject through the head by an irrigation device, and the head further comprises a needle in fluid communication with and extending from the head of the instrument, where prior to delivering fluid from the fluid reservoir, the method further comprises removing fluid and material from the body of the subject through the head by a suction device; piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument. In some embodiments, the method further comprises moving fluid into the body of the subject through the head by an irrigation device. In some embodiments, the head is at least one of configured to enable fluid and material to be removed from the body of a subject through the head by a suction device; and configured to enable fluid to move into the body of a subject through the head by an irrigation device, and the head further comprises a needle in fluid communication with and extending from the head of the instrument, where prior to delivering fluid from the fluid reservoir, the method further comprises moving fluid into the body of the subject through the head by an irrigation device; piercing a surface layer of tissue of the target area of the subject with the needle to enable fluid from the fluid reservoir to be delivered through tissue of the target area with the instrument; and delivering fluid from the fluid reservoir, through the shaft, through the needle, and through tissue of the target area with the instrument.

In some embodiments, the methods comprise creating at least one incision in a body of a subject; disposing at least a portion of a trocar within the at least one incision to provide access to a target area within the body of the subject; disposing at least a portion of an instrument within the trocar such that the instrument can access the target area within the body of the subject, where the instrument comprises: a head; a shaft in fluid communication with a fluid reservoir, where the fluid reservoir comprises fluid; and where the instrument is configured to permit delivery of fluid from the fluid reservoir, through the shaft, and through tissue of the subject; where the instrument is configured to permit removal of fluid from the target area of a subject through tissue of the subject; and where the instrument is configured to be coupled to a platform for minimally invasive procedures; removing fluid from the target area of a subject through tissue of the subject; and delivering fluid from the fluid reservoir, through the shaft, and through tissue of the target area with the instrument. In some embodiments, at least a portion of the fluid in the fluid reservoir is fluid that is detectable within the body with a viewing device. In some embodiments, the instrument further comprises a pump configured to pump fluid from the fluid reservoir through the shaft, where delivering fluid comprises pumping fluid, with the pump, from the fluid reservoir, through the shaft, and through tissue of the target area with the instrument.

In some embodiments, the methods further comprise creating at least one incision in a body of a subject; disposing at least a portion of a trocar within the at least one incision to provide access to a target area within the body of the subject; disposing at least a portion of an instrument within the trocar such that the instrument can access the target area within the body of the subject, where the instrument comprises a head; and a shaft in fluid communication with a fluid reservoir, where the fluid reservoir comprises fluid, and at least a portion of the fluid in the fluid reservoir is medication (or serves a medical benefit or purpose); where the instrument is configured to permit delivery of fluid from the fluid reservoir, through the shaft, and through tissue of the subject; delivering fluid from the fluid reservoir, through the shaft, and through tissue of the target area with the instrument.

In some embodiments, the methods for minimally invasive procedures comprise creating at least one incision in a body of a subject; disposing at least a portion of a trocar within the at least one incision to provide access to a target area within the body of the subject; coupling an instrument to a platform for minimally invasive procedures to enable an operator to control the instrument; disposing at least a portion of the instrument within the trocar such that the instrument can access the target area within the body of the subject, where the instrument comprises: a head in fluid communication with a needle; and a shaft in fluid communication with the head and further in fluid communication with a fluid reservoir, where the fluid reservoir comprises fluid, and at least a portion of the fluid in the fluid reservoir is medication; where the instrument is configured to permit delivery of fluid from the fluid reservoir, through the shaft, through the needle, and into the target area of the subject; delivering fluid from the fluid reservoir, through the shaft, through the needle, and into the target area of the subject with the instrument. In some embodiments, the method further comprises repairing a hernia with the platform for minimally invasive procedures. In some embodiments, the method further comprises activating a pump, with the platform for minimally invasive procedures, to pump fluid from the fluid reservoir and into the target area of the subject with the instrument.

In some embodiments, the methods for minimally invasive procedures comprise identifying a target area within a body of a subject; identifying an incision site that is separate from and does not overly the target area; creating at least one incision in the body of the subject at the incision site; disposing at least a portion of a trocar within the at least one incision to provide access to the target area; coupling an instrument to a platform for minimally invasive procedures to enable an operator to control the instrument; disposing at least a portion of the instrument within the trocar to enable an operator to access the target area with the instrument, where the instrument comprises: a needle in fluid communication with a fluid reservoir, where the fluid reservoir comprises fluid, and at least a portion of the fluid in the fluid reservoir is medication; where the instrument is configured to permit delivery of fluid from the fluid reservoir, through the needle, and into the target area; piercing the target area with the needle; and delivering fluid from the fluid reservoir, through the needle, and into the target area. In some embodiments, at least a portion of the fluid in the fluid reservoir is fluid that is detectable within the body with a viewing device, and the method further comprises detecting the fluid that is detectable within the body with a viewing device; and determining whether sufficient fluid has been delivered to the target area, based on an amount of fluid detected with the viewing device.

Exemplary Procedures

By way of example, and not limitation, the following minimally invasive surgical procedures are described to demonstrate several ways in which the present systems, instruments, and methods can be implemented to, among other things, achieve the advantages described in this disclosure. The following examples should not be understood to limit the potential uses of the disclosed systems, instruments, and methods, but instead, should be understood to illustrate some ways in which such systems, instruments, and methods can be implemented in practice.

A first example is provided for surgical repair of a hernia/defect/opening in the abdominal wall, in which the musculature, surrounding fascia, and structure of the abdominal wall is directly or indirectly manipulated by instruments coupled to a platform for minimally invasive procedures. Such a procedure can include reduction of incarcerated contents, debridement or liberation of attachments, structures, and/or tissue, closure (re-approximation, coaptation, and the like) or near-closure of the hernia/defect/opening, and/or fixation of mesh or prosthetic to the abdominal wall. One or more incisions can be created in a portion of the body that does not overly the hernia/defect/opening. For example, one or more incisions can be created over the lateral abdominal wall, if the hernia/defect/opening is within a substantially more medial area of the abdominal wall, such as in the vicinity of the rectus abdominal muscle; one or more incisions can be created over the lateral abdominal wall, if the hernia/defect/opening is within an area substantially near to the contra-lateral abdominal wall; and one or more incisions can be created over the medial area of the abdominal wall, if the hernia/defect/opening is within a substantially more lateral area of the abdominal wall. One or more trocars can be disposed within the one or more incisions to provide access to the hernia/defect/opening. For example, a viewing device can be disposed within one of the trocars to provide visual access to the hernia/defect/opening; and an instrument of this disclosure can be disposed within another of the trocars to enable an operator to access the hernia/defect/opening with the instrument. Using the systems and instruments of this disclosure, fluid, including medication, can be injected directly into the abdominal wall from the domain of the internal abdominal cavity, as opposed to breaching the skin barrier in transcutaneous delivery of fluid to the abdominal wall, in a surgical procedure in which the one or more incisions do not overly the hernia/defect/opening. Medication can include anesthetic or pain-receptor-modulating agents to, for example, treat and/or prevent pain experienced by the subject undergoing the procedure. Delivery of medication into the abdominal wall using the systems and methods of this disclosure can include delivery of medication to substantially all areas that could induce pain in the subject during or after the procedure, including at hernia/defect/opening suturing sites, mesh suturing sites, incision suturing sites, and a reasonable perimeter of the portion of the abdominal wall that is the subject of a given procedure. In addition, fluid in the systems and instruments of this disclosure can include an agent that is detectable by a viewing device to enable an operator to visualize the locations into which the fluid has been delivered. This can include, but is not limited to, indocyanine green, which can be observed by a fluorescence laparoscopic camera lens. For example, a mixture of medication and indocyanine green can be injected with the systems and instruments of this disclosure into the abdominal wall (e.g., in the vicinity of a hernia/defect/opening and in the vicinity of any suturing of the hernia/defect/opening and suturing of any mesh (e.g., at the discretion of an operator)) from the domain of the abdominal cavity. A viewing device disposed within one of the trocars to provide visual access to the hernia/defect/opening can include a fluorescent lens of a camera that can detect indocyanine green that has been dispensed from the systems and instruments to enable an operator to determine whether sufficient medication has been delivered to the necessary and/or desired areas to obtain the desired anesthetic effect, such as sufficiently reducing pain during and after the procedure. More fluid can be delivered at the discretion of the operator. Delivery of fluid from the internal domain of an abdominal cavity can improve accuracy and reliability, for example, as compared to multiple transcutaneous puncture sites, which can prove inaccurate due to increased variability in amount of subcutaneous tissue to penetrate and can increase a possibility of contaminating prosthetic mesh (e.g., with bacteria from the skin surface), resulting in a potential mesh infection.

A second example is provided for partial or complete removal of a gallbladder with a minimally invasive procedure. Such a procedure can include instances in which distension of the gallbladder interferes with grasping and manipulation of the organ. One or more incisions can be created in a portion of the body, and one or more trocars can be disposed within the one or more incisions to provide access to the gallbladder. For example, a viewing device can be disposed within one of the trocars to provide visual access to the gallbladder; and an instrument of this disclosure can be disposed within another of the trocars to enable an operator to access the gallbladder with the instrument. Using the systems and instruments of this disclosure, the gallbladder can be punctured to gain access to the lumen of the gallbladder in order to evacuate fluid from the lumen of the distended organ, which can prevent contamination of the abdominal cavity. As with example 1 above, using the systems and instruments of this disclosure, fluid comprising an agent that is detectable by a viewing device, such as indocyanine green, can be injected into the gallbladder and into the biliary duct system to enable an operator to visualize the biliary anatomy in order to facilitate improved dissection of the intended anatomy and prevent inappropriate manipulation or injury to structures that should be preserved. In such an example, the structures that can be identified and preserved include, for example, the common hepatic duct, the common bile duct, the right hepatic artery. Delivery of fluid in this manner decreases the time to visualize the biliary anatomy, for example, as compared to intravenous injection of indocyanine green, which can require greater than 45 minutes. Additionally, delivery of fluid in this manner provides increased contrast of the gallbladder and the biliary system to improve visualization, for example, as compared to intravenous injection of indocyanine green, which can obscure visualization.

A third example is provided for surgically removing a segment of intestine, such as in the case of an intestinal pathology (e.g., due to malignancy, fistula, or other disease), relieving pressure due to an intestinal obstruction (e.g., adhesive or benign mechanical), testing for a leak in the intestines, and/or testing the integrity of a surgically-created anastomosis. The systems and methods of this disclosure can be used in open laparotomy procedures or minimally invasive procedures. Such procedures can include instances in which intestinal organs are manipulated, resected, decompressed, breached, and/or diverted, and there is a risk of contamination of the intra-abdominal space with the enteric contents. For example, a procedure in which an intestinal obstruction is addressed surgically and may require a stapled occlusion at a distal point and a proximal point of resection, followed by creation of an enterotomy through each arm of the intestines to be connected, and any technique described to perform an intestinal anastomosis. Enteric contents may accumulate under pressure on either side of the portion of the intestine that is being removed. Prior to beginning the anastomosis procedure, the systems and instruments of this disclosure can be used to drain fluid buildup within the intestines by puncturing the intestinal wall and removing fluid by aspiration. As described in detail in the above disclosure, the present needles can be coupled to an instrument such that it is in fluid communication with a suction/aspiration device to enable an operator to aspirate fluid from within the intestine in a controlled manner without exposing the intestinal contents to the abdominal cavity. Controlled drainage of the enteric contents prior to the anastomosis procedure can prevent the expulsion of enteric contents into the otherwise sterile or clean environment of the abdominal cavity. Similarly, during a procedure in which the intestines are not resected, but an intestinal obstruction is being relieved (e.g., a mechanical obstruction, internal obstruction, adhesive obstruction, and the like), the enteric contents may accumulate under pressure on either side of the obstruction. To relieve pressure in the system and facilitate decompression of the intestines, the systems and instruments of this disclosure can be used to drain fluid buildup within the intestines by puncturing the intestinal wall and removing fluid and subsequently the pressure and distension within the intestine. This puncture/aspiration procedure can be implemented at any point along the intestine and as many times as necessary to decompress the intestines. Finally, the systems and instruments of this disclosure can be used to test whether a leak is present in the intestines (e.g., such as a naturally-occurring leak, a leak created during a procedure, and/or a leak arising from a surgically-created anastomosis) by, for example, temporarily occluding the distal segment and proximal segment of the area in question, picturing the intestine with a needle, injecting fluid comprising an agent that is detectable by a viewing device, such as indocyanine green, into the intestines and observing whether such fluid exits the intestine using a viewing device. Presence or absence of fluid can determine whether the anastomosis may be leaking. Alternatively, an area in question can be clamped distally and proximally, submerged under a layer of fluid within the abdominal cavity, and a pulse of air can be injected into the lumen of the intestine with a needle of the present instruments. Presence or absence of air bubbles can determine whether the anastomosis may be leaking.

A fourth example is provided for identifying and removing sentinel lymph nodes during an operation involving a suspected or confirmed malignancy. As described in other examples in this disclosure, the systems and instruments can be used to facilitate identification of a sentinel lymph node from within the abdominal cavity by accessing and injecting the tissue in proximity to the malignancy or lesion in question that is positioned within the abdominal cavity and, for example, which could not otherwise be easily accessed without the use of a colonoscope, esophagogastroduodenoscope, colposcopy, and/or other endoscopic procedural device through a natural orifice of the body. While in the intra-abdominal cavity, or in the subcutaneous tissue, the tissue in proximity to a malignant lesion can be injected by the systems and instruments with fluid, such as fluid containing a combination of medication and agent, such as indocyanine green, that is detectable by a viewing device, such as a fluorescent filter of a camera, that will permeate the surrounding tissue and perfuse the lymphatic or vascular system. The fluid is expected to reach the sentinel lymph node, which can then be distinguished from other lymph nodes (e.g., those that are not sentinel lymph node) by viewing and identifying the peak intensity of uptake of the detectable fluid within such a sentinel lymph node. Excision of this sentinel lymph node and resection of the malignancy or lesion can then be performed.

The above specification and examples provide a description of the structure and use of exemplary embodiments. Although embodiments have been described above with a certain degree of particularity, or with reference to one or more individual embodiments, those skilled in the art could make numerous alterations to the disclosed embodiments without departing from the scope of this invention. As such, the various illustrative embodiments of the present instruments, systems, and methods are not intended to be limited to the particular forms disclosed. Rather, they also include all combinations, integrations, omissions, modifications, equivalents, and alternatives falling within the scope of the claims, and embodiments other than the ones shown may include some or all of the features of the depicted embodiment. For example, components may be combined as a unitary structure and/or connections may be substituted. The scope of protection is not limited by the description set out above but is defined by the claims that follow, including all equivalents of the subject matter of the claims. Further, where appropriate, aspects of any of the embodiments described above may be combined with aspects of any of the other embodiments described to form further embodiments having comparable or different properties and addressing the same or different problems. Similarly, the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An instrument, comprising:
a head configured to be coupled to a needle;
a shaft comprising a lumen, the shaft in fluid communication with the head, where the shaft is configured to be in fluid communication with a fluid reservoir, and where the head comprises at least two degrees of freedom with respect to the shaft throughout a procedure;
a housing in fluid communication with the shaft;
where at least a portion of the instrument is configured to be disposed through a trocar into a body of a subject;
where the instrument is configured to be coupled to a platform for minimally invasive procedures and controllable by an operator of the platform for minimally invasive procedures;
where, when at least a portion of the instrument is being disposed through a trocar into the body of the subject, the head and the shaft are substantially colinear, and when the head exits the trocar, the head is configured to be movable from within the shaft such that the head is non-colinear with respect the shaft; and
where, if the instrument is coupled to a fluid reservoir and at least a portion of the instrument is disposed through a trocar into the body of the subject, and if the needle is coupled to the head, tissue of the subject can be pierced by the needle to permit delivery of fluid through the tissue.

2. The instrument of claim 1, further comprising a needle configured to be coupled to the head in fluid communication with the shaft, where the needle comprises a first end and a second end, where the second end is configured to extend from the head of the instrument, and the first end comprises an end opening, where the needle further comprises at least one opening between the second end and the first end, and the end opening and the at least one opening are configured to permit the fluid to exit the needle.

3. The instrument of claim 1, where the housing comprises a pump, and where, if the instrument is coupled to a fluid reservoir, the pump is configured to pump fluid from the fluid reservoir through the shaft and into the head.

4. The instrument of claim 3, where the pump comprises a positive displacement pump, and the pump is configured to and controllable to permit a predetermined amount of fluid into the shaft.

5. The instrument of claim 1, further comprising a fluid reservoir configured to be coupled to the instrument, where at least a portion of the fluid in the fluid reservoir is anesthetic.

6. The instrument of claim 5, where at least another portion of the fluid in the fluid reservoir comprises fluid that, if delivered through the tissue of the subject, is detectable within the body with a viewing device.

7. The instrument of claim 1, where, if the instrument is coupled to a platform for minimally invasive procedures and controllable by an operator of the platform for minimally invasive procedures, the instrument is configured to permit the operator to deliver the fluid through a surface layer of tissue of the subject and into at least a portion of an abdominal wall of the subject.

8. The instrument of claim 1, where, if the instrument is coupled to a platform for minimally invasive procedures and controllable by an operator of the platform for minimally invasive procedures, the instrument is configured to permit an operator to deliver fluid through a surface layer of tissue of an organ of a subject and into a lumen of the organ, and the instrument is further configured to permit an operator to remove fluid through a surface layer of tissue of a subject.

9. An instrument, comprising:
a head configured to be coupled to a needle;
a fluid reservoir in which at least a portion of fluid in the fluid reservoir is anesthetic;
a shaft comprising a lumen, the shaft coupled to and in fluid communication with the head, where the shaft is configured to be in fluid communication with the fluid reservoir in which at least a portion of the fluid in the fluid reservoir is anesthetic, and where the head is configured to be movable with respect to the shaft;
a housing in fluid communication with the shaft;
where at least a portion of the instrument is configured to be disposed through a trocar into a body of a subject;
where the instrument is configured to be coupled to a platform for minimally invasive procedures and controllable by an operator of the platform for minimally invasive procedures; and
where, if the shaft is in fluid communication with the fluid reservoir, and at least a portion of the instrument is disposed through a trocar into the body of the subject, the instrument is configured to permit delivery of the fluid through tissue of the subject.

10. The instrument of claim 9, where the housing comprises a fluid reservoir that is in fluid communication with the shaft.

11. The instrument of claim 10, where the housing comprises a pump that is configured to pump fluid from the fluid reservoir through the shaft and into the head.

12. The instrument of claim 9, where the housing is configured to be coupled to the fluid reservoir that is in fluid communication with the shaft.

13. The instrument of claim 12, where, if the housing is coupled to the fluid reservoir such that the fluid reservoir and the shaft are in fluid communication, the fluid can move from the fluid reservoir into the shaft by at least one of gravity and by applying manual pressure.

14. The instrument of claim 9, where, if the instrument is coupled to a platform for minimally invasive procedures and controllable by the operator of the platform for minimally invasive procedures, the instrument is configured to permit the operator to deliver the fluid through a surface layer of tissue of the subject and into at least a portion of an abdominal wall of the subject.

15. An instrument, comprising:
a head;
a shaft coupled to the head and comprising a lumen, the shaft in fluid communication with the head, where the shaft is configured to be coupled to and in fluid communication with a fluid reservoir;
where the head and the shaft are coupled such that, if a force is applied to the head, the head remains in a substantially similar position;
where at least a portion of the instrument is configured to be disposed through a trocar into a body of a subject;
where the instrument is configured to be coupled to a platform for minimally invasive procedures and controllable by an operator of the platform for minimally invasive procedures such that the operator can control the instrument to permit delivery of fluid through tissue of a subject; and
where, if the instrument is coupled to a fluid reservoir and at least a portion of the instrument is disposed through a trocar into the body of the subject, the instrument is configured to permit delivery of the fluid through the tissue of the subject.

16. The instrument of claim 15, where, if the instrument is coupled to a platform for minimally invasive procedures, the platform for minimally invasive procedures is configured to permit an operator to deliver a predetermined amount of the fluid through at least a portion of the instrument.

17. The instrument of claim 16, where, if the instrument is coupled to a platform for minimally invasive procedures and controllable by the operator of the platform for minimally invasive procedures, the operator can control the instrument to permit delivery of the fluid through a surface layer of tissue of the subject and into at least a portion of an abdominal wall of the subject, and where, if the instrument is coupled to a fluid reservoir, at least a portion of the fluid in the fluid reservoir is anesthetic.

18. The instrument of claim 16, where the head is configured to be at least one of rotatable with respect to the shaft while the head and the shaft remain substantially colinear and movable with respect to the shaft, where, if the instrument is coupled to a platform for minimally invasive procedures and controllable by the operator of the platform for minimally invasive procedures, the operator can at least one of rotate and move the head with respect to the shaft.

19. The instrument of claim 16, where the instrument is coupled to a pump, such that if the instrument is coupled to a platform for minimally invasive procedures and controllable by the operator of the platform for minimally invasive procedures, and if the instrument is coupled to a fluid reservoir, the pump is configured to be activated by the operator to permit delivery of the fluid through the tissue of the subject.

20. The instrument of claim 19, where, if the instrument is coupled to a platform for minimally invasive procedures and controllable by the operator of the platform for minimally invasive procedures, the instrument is configured to be activated by the operator to permit delivery of fluid through a surface layer of tissue of the subject and into at least a portion of an abdominal wall of the subject without breaching the skin barrier in transcutaneous delivery of the fluid to the abdominal wall, and where, if the instrument is coupled to a fluid reservoir, at least a portion of the fluid in the fluid reservoir is anesthetic.
